(19) United States
(12) Patent Application Publication
(10) Pub. No.: US 2023/0113944 A1
(43) Pub. Date: Apr. 13, 2023

(54) COMPOSITIONS AND METHODS FOR THE TREATMENT OF NEURODEGENERATIVE DISEASES

(71) Applicant: The Regents of the University of California, Oakland, CA (US)

(72) Inventors: Varghese John, Los Angeles, CA (US); Tina Bilousova, Santa Monica, CA (US); Bryan Simmons, Santa Clarita, CA (US); Neil Garg, Los Angeles, CA (US); Jesus Campagna, Playa Del Rey, CA (US); Barbara Jagodzinska, Redwood City, CA (US)

(21) Appl. No.: 17/790,407

(22) PCT Filed: Jan. 8, 2021
(86) PCT No.: PCT/US21/12643

§ 371 Date: Jun. 30, 2022

Related U.S. Application Data
(60) Provisional application No. 62/959,578, filed on Jan. 10, 2020.

(51) Int. Cl. C07D 491/147; A61P 27/06; A61P 25/28; C07D 491/048

(52) U.S. Cl. CPC C07D 491/147; A61P 27/06; A61P 25/28; C07D 491/048

(57) ABSTRACT

Provided herein are compounds of Formula I and their salts, and compositions thereof that are useful for useful for modulating neutral sphingomyelinase 2 (n-SMase2) and/or acetylcholinesterase (AChE) in cells. Also disclosed herein are methods of using the disclosed compounds and compositions for inhibiting the spread of Tau seeds from donor cells to recipient cells. Further disclosed herein are methods of using the disclosed compounds and compositions for treating or preventing a neurodegenerative disorder, such as a tauopathy, Alzheimer's disease (AD), Parkinson's disease (PD), Huntington's disease (HD), Lewy body dementia, frontotemporal dementia, and amyotrophic lateral sclerosis (ALS).

### RELATED APPLICATIONS

This application claims the benefit of U.S. Provisional Patent Application No. 62/959,578, filed on Jan. 10, 2020, the contents of which are hereby incorporated by reference in their entirety.

### STATEMENT OF GOVERNMENT SUPPORT

This invention was made with government support under Grant Number AG051386, awarded by the National Institutes of Health, and Grant Number 1464898, awarded by the National Science Foundation. The government has certain rights in the invention.

### BACKGROUND

A neurodegenerative disease is an umbrella term for the progressive degeneration of neurons in, e.g., the central nervous system (CNS), characterized by molecular and genetic changes in nerve cells that result in nerve cell degeneration and ultimately nerve dysfunction and death. Neurodegenerative diseases affect an estimated 50 million Americans each year, exacting an incalculable personal toll and an annual economic cost of hundreds of billions of dollars in medical expenses and lost productivity. Neurodegenerative diseases include, but are not limited to, tauopathies, Alzheimer's disease (AD), amyotrophic lateral sclerosis (ALS), Huntington's disease (HD), and Parkinson's disease (PD).

At present, there are no effective treatments for halting, preventing, or reversing the progression of such neurodegenerative diseases. Therefore, there is an urgent need for pharmaceutical agents capable of slowing the progression of the aforementioned neurodegenerative diseases (and others) and/or preventing them in the first place.

### SUMMARY OF THE INVENTION

The present disclosure provides compounds of Formula I:

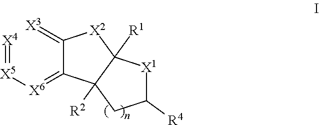

or a pharmaceutically acceptable salt thereof, wherein:
X¹ and X² are each independently O or NR^{a};
X³, X⁴, X⁵, and X⁶ are each independently CR³ or N, preferably selected such that no more than two of X³, X⁴, X⁵, and X⁶ are N;
R¹ is hydrogen or alkyl, preferably hydrogen or lower alkyl, most preferably hydrogen;
R² is alkyl, haloalkyl, alkoxy, cycloalkyl, aryl, aralkyl, or haloalkyl, preferably lower alkyl;
each R³ is independently hydrogen, halogen, —OC(O)NR^{b}R^{c}, alkyl, haloalkyl, alkoxy, aralkyloxy, cycloalkyl, aryl, or heteroaryl, preferably such that at least one R³ (most preferably the one on X⁵) is selected from halogen, —OC(O)NR^{b}R^{c}, alkyl, haloalkyl, alkoxy, aralkyloxy, cycloalkyl, aryl, or heteroaryl;
R⁴ is hydrogen or alkyl;
each R^{a} is independently hydrogen, alkyl, —C(O)-alkyl, —S(O)₂-alkyl, or —S(O)₂-aryl, preferably H or lower alkyl;
R^{b} is hydrogen, alkyl, cycloalkyl, aryl, heteroaryl, or heterocyclyl, preferably aryl (e.g., phenyl) or heteroaryl (e.g., pyrid-4-yl);
R^{c} is hydrogen or alkyl, preferably hydrogen; and
n is an integer from 1 to 4, preferably 1 or 2.

In one aspect, the invention provides pharmaceutical compositions of compounds disclosed herein, e.g., that comprise a pharmaceutically acceptable excipient and a compound of Formula I, or a pharmaceutically acceptable salt thereof. The pharmaceutical compositions can be used in therapy, e.g., for treating a disease or condition disclosed herein in a subject.

In another aspect, provided herein are methods for modulating neutral sphingomyelinase 2 (N-SMase2) in a cell, comprising contacting a cell with a compound of Formula I or a pharmaceutically acceptable salt thereof, or a pharmaceutical composition as disclosed herein. In some embodiments, the cell occurs in a subject, and the method serves to treat an N-SMase2-mediated condition and/or disease. In certain preferred embodiments, the method also modulates acetylcholinesterase (AChE). In some such embodiments, the cell occurs in a subject, and the method serves to treat an AChE-mediated condition and/or disease. In some preferred embodiments, the cell occurs in a subject, and the method serves to treat a condition and/or disease mediated by both N-SMase2 and AChE.

In yet another aspect, provided herein are methods for inhibiting the spread of Tau seeds from donor cells to recipient cells, comprising contacting the cells with a compound of Formula I or a pharmaceutically acceptable salt thereof, or a pharmaceutical composition as disclosed herein. In some embodiments, the cell occurs in a subject, and the method serves to treat a condition and/or disease associated with Tau deposits.

In yet another aspect, provided herein are method for treating or preventing a disease or disorder associated with accumulation and/or aggregation of misfolded proteins, comprising administering to a subject in need thereof a therapeutically effective amount of a compound of Formula I, or a pharmaceutically acceptable salt thereof, or a pharmaceutical composition as disclosed herein.

In another aspect, provided herein are methods for treating or preventing a neurodegenerative disease or disorder, comprising administering to a subject in need thereof a therapeutically effective amount of a compound of Formula I or a pharmaceutically acceptable salt thereof, or a pharmaceutical composition as disclosed herein. In some embodiments, the neurodegenerative disorder is selected from a tauopathy, Alzheimer's disease, Parkinson's disease, Huntington's disease, Lewy body dementia, frontotemporal dementia, and amyotrophic lateral sclerosis.

### BRIEF DESCRIPTION OF THE DRAWINGS

FIG. 1 depicts a bar graph showing the inhibition of N-SMase2 activity in the presence of certain compounds of the invention.

FIG. 2A depicts inhibition of nSMase2 by DDL-122 and DDL-133 (+/−).

FIG. 2B depicts inhibition of AChE by DDL-122 and DDL-133 (+/−). In this assay the control, donepezil, possess an IC₅₀ of approximately 0.25 uM (not depicted).

FIG. 3A depicts the binding free energy calculation of the binding of DDL-133 to the DK-switch and SM binding site.

FIG. 3B depicts the preferential binding mode of DDL-133 to the sphingomyelin (SM) binding site.

FIG. 4A depicts Tau biosensor cells that were seeded with AD human brain derived synaptosomal (P2) extract and grow in presence of 10 uM of nSMase2 inhibitors or corresponded amount of DMSO. Cells were imaged in 24 hrs after seeding.

FIG. 4B depicts Tau biosensor cells that were seeded with AD human brain derived synaptosomal (P2) extract and grow in presence of 10 uM of nSMase2 inhibitors or corresponded amount of DMSO. After 60 hrs cell culture medium and cells were collected. Cells were fixed and FRET signal was analyzed using flow cytometry.

FIG. 4C depicts Tau biosensor cells that were seeded with AD human brain derived synaptosomal (P2) extract and grow in presence of 10 uM of nSMase2 inhibitors or corresponded amount of DMSO. Cell culture medium was used for extracellular vesicle (EV) purification using ExoQuick methods. Amount of exosomal marker (CD63) in the purified EV fractions was assessed by Western blotting analysis.

FIG. 5 shows that DDL-133 inhibits nSMase 2 with an IC₅₀ of approximately 0.5 μM.

FIG. 6A shows the results of a screen for nSMase2/AChE inhibitors. The nSMase2 inhibitor screening using an Amplex Red-coupled assay revealed 5 hits that inhibited activity ≥60%

FIG. 6B depicts dose-response curves for compounds 1 (phensvenine), 8, and 11 in the nSMase2 activity assay show 1 has nSMase2 inhibitory activity, but 8 and 11 are more potent.

FIG. 6C depicts that in the AChE activity assay, dose-response curves reveal 1 was the most potent, followed by 11 and 8.

FIG. 6D shows the structure-activity relationship (SAR) control elements for inhibition of nSMase2 and AChE activity.

FIG. 6E shows a summary of the hit-to-lead optimization strategy. The removal of the nitrogen group from furoindoline ring and addition of nitrogen to the carbamate phenyl ring at either the 3 or 4 positions modulates nSMase2 and AChE activity.

FIG. 7A depicts the kinetics of enzymatic inhibition of nSMase2 by compound 8. The rate of the reaction is plotted against substrate concentration at four different concentrations of the inhibitor; corresponding values for Vₘₐₓ and Kₘ are presented in the tables below the graphs

FIG. 7B depicts the kinetics of enzymatic inhibition of nSMase2 by compound 11. The rate of the reaction is plotted against substrate concentration at four different concentrations of the inhibitor; corresponding values for Vₘₐₓ and Kₘ are presented in the tables below the graphs

FIG. 8A shows that Dual nSMase2/AChE inhibitors 8 and 11 suppress tau propagation from donor to recipient cells in vitro. Donor plus recipient (D+R) assay results are shown. Compounds 8 and 11 at a concentration of 20 μM or a corresponding volume of DMSO were added to the D+R cultures for 48 hrs. Levels of FRET signal were analyzed in recipient cells using flow cytometry. Combined data from three independent experiments is presented.

FIG. 8B shows that Dual nSMase2/AChE inhibitor 8 and 11 suppress tau propagation from donor to recipient cells in vitro. EV-mediated tau seed transfer (EMT) assay results are shown. Compounds 8 and 11 at 20 μM concentration or DMSO were added to donor cell culture medium and then donor cell-derived EVs were purified and transfected to recipient cells.

FIG. 8C shows levels of FRET signal which were analyzed in recipient cells using flow cytometry. Four technical replicates were used for each experimental condition. Combined data from three independent experiments is presented. The histograms represent integrated FRET density per each treatment group (mean±SEM). Size distribution and concentrations of the donor-derived EV samples were analyzed by Tunable Resistive Pulse Sensing (TRPS). The figure also depicts donor-derived EVs which were imaged using transmission electron microscopy (TEM).

FIG. 8D shows western blot representative images for exosomal markers. The same volume of EV fractions derived from a similar number of donor cells or control tau biosensor cells treated with Lipofectamine 2000 (LIPO-control) were loaded per well and probed against exosomal markers CD63, CD81, and Syntenin-1. Statistics were performed using One-way ANOVA with post hoc Bonferroni and Holm multiple comparison test was used for statistical analysis: * p<0.05, **<0.01.

FIG. 9A depicts a pharmacokinetic analysis for compounds 8 and 11. Mice were subcutaneously (SQ) injected with 20 mg/kg of compound 8 or 11; animals were sacrificed 1, 2, and 4 hours after dosing (n=1 animal per time point). Compound levels in brain tissue were analyzed using an LC-MS/MS method. Both compounds penetrated the brain.

FIG. 9B depicts a pharmacokinetic analysis for compounds 8 and 11. Mice were subcutaneously (SQ) injected with 20 mg/kg of compound 8 or 11; n=6 per compound and sacrificed 1 hour after dosing. Compound levels in brain tissue were analyzed using an LC-MS/MS method. Both compounds penetrated the brain.

FIG. 10A shows the size distribution and concentrations of the brain EV samples that were analyzed by Tunable Resistive Pulse Sensing (TRPS). Dual nSMase2/AChE inhibitor 11 diminished IL1β-induced brain EV release in the rapid in vivo assay. Tau P301S (line PS19) mice were treated with compound 8 or 11 subcutaneously (SQ) at 20 mg/kg one hour before IL1β injection (unilateral ICV injection of 0.2 ng). Two hours after IL1β injection, brain tissue was collected and used for brain EV isolation.

FIG. 10B shows the average concentrations of 50-150 nm size EVs from each treatment condition.

FIG. 10C depicts a representative transmission electron microscopy (TEM) image of the brain EV fraction.

FIG. 10D depicts representative images of western blot (WB) analysis of EV fractions from individual animals is shown; membranes were probed against exosomal markers (CD63 and syntenin-1), tau protein, and cell-type specific markers (astrocytic glutamate-aspartate transporter GLAST1, microglia marker CD11b, and neuronal isoform of Bridging Integrator 1, BIN1).

FIG. 10E depicts the densitometry analysis of the WB images. Histograms represent average relative signal intensity per each treatment group (mean±SEM). Statistical analysis was performed using one-way ANOVA with post hoc Bonferroni and Holm multiple comparison tests: #-P<0.05 and ##-P<0.01 compared to control group, treated with vehicles for SQ and ICV injections, *-P<0.05 and **-P<0.01 compared to IL1 (3 group.

FIG. 11 depicts a putative mechanism for dual nSMase2/AChE inhibition and suppression of EV/exosome-mediated propagation of tau pathology wherein nSMase2 inhibition suppresses exosome biogenesis while AChE inhibition reduces exosome uptake and cholinergic support.

FIG. 12A depicts the flow cytometry analysis of donor cells. In particular, representative dot-plots show the FRET signal (gated events) in tau biosensor cells treated with transfection reagent Lipofectamine 2000 (LIPO-control).

FIG. 12B depicts the flow cytometry analysis of donor cells. In particular, representative dot-plots show the FRET signal (gated events) in donor cells transfected with synaptosomal extract from human AD brain (P2).

FIG. 12C depicts the flow cytometry analysis of recipient cells and recipient cells transfected with EVs purified from cell culture medium from LIPO-control cells.

FIG. 12D depicts the flow cytometry analysis of recipient cells and recipient cells transfected with EVs purified from cell culture medium from P2-seeded donor cells treated with DMSO.

FIG. 12E depicts the flow cytometry analysis of recipient cells and recipient cells transfected with EVs purified from cell culture medium from P2-seeded donor cells treated with 20 μM of compound 8.

FIG. 12F depicts the flow cytometry analysis of recipient cells and recipient cells transfected with EVs purified from cell culture medium from P2-seeded donor cells treated with 20 μM of compound 11.

FIG. 13A shows cell viability in D+R and EMT assays. In particular, the number of LIPO-control donor cells after 48-hour treatment with 20 μM of compounds 8, 11, or DMSO in a EMT assay are displayed.

FIG. 13B shows cell viability in D+R and EMT assays. In particular, levels of lactate dehydrogenase (LDH) in the medium collected from donor cells described in from FIG. 13A are presented as a percentage from LDH in LIPO-control cultures.

FIG. 13C shows cell viability in D+R and EMT assays. In particular, the level of LDH released in recipient cells is shown.

FIG. 13D shows the cell viability in D+R and EMT assays. In particular, LDH levels in cell culture medium in the D+R assay (48-hour treatment with 20 μM of compounds 8, 11, or DMSO) are shown.

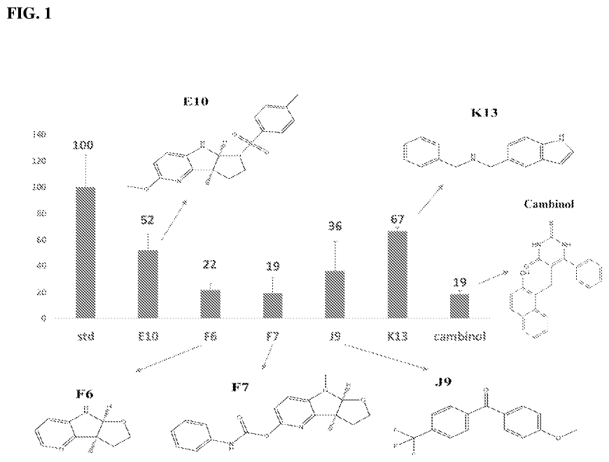

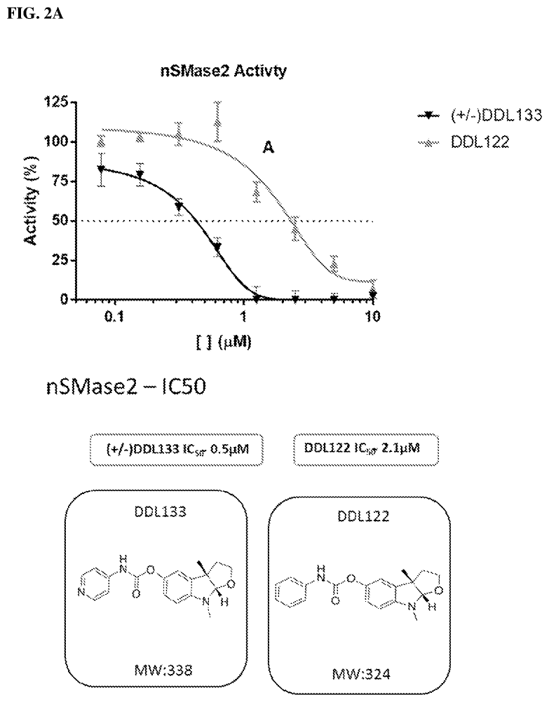

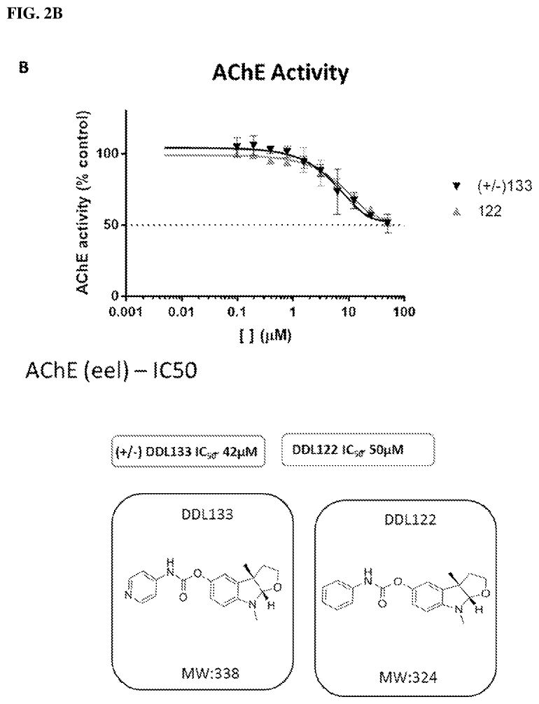

### DETAILED DESCRIPTION OF THE INVENTION

Alzheimer's disease (AD) is the most prevalent age-related neurodegenerative disorder and currently no effective disease-modifying therapy is available. The number of AD cases in the US is increasing rapidly, affecting ˜5.8 million patients currently with this number expected to rise to 50 million by 2050. The estimated global socioeconomic costs of AD and related dementias are predicted to reach S2 trillion by the year 2030. AD brain tissue is characterized by the presence of senile plaques composed mainly of aggregated amyloid-3 peptide (Aβ), neurofibrillary tangles (NFTs) composed of pathological forms of the microtubule-stabilizing protein tau, chronic neuroinflammation, and loss of neurons. Clinically, it is thought that the underlying mechanisms of disease are initiated as long as 20 years before the onset of signs and symptoms. During this asymptomatic period, proteopathic proteins are believed to accumulate leading to structural alterations and the neuronal dysfunction and loss that leads frequently first to Mild Cognitive impairment (MCI); MCI then progresses to full-blown AD-related memory deficits, decline of other cognitive skills, and—in advanced AD—the inability to participate in activities of daily living.

While the exact mechanisms of disease progression have not been fully elucidated, it is thought that increased Aβ production at the synapse and/or impaired clearance results in synaptic loss. Contemporaneously and in conjunction with Aβ accumulation, there is hyperphosphorylation and oligomerization of tau that eventually leads to neuronal toxicity, NFT formation, and cell death. Diseased neurons can release these toxic phosphorylated forms of tau (p-tau) in proteopathic seeds, which can then in turn be taken up by surrounding or interconnected neurons leading to templating and propagation of the pathological aggregates in prion-like fashion.

The propagation of disease follows a spatiotemporal pattern. AP plaques first appear in the basal forebrain, then the frontal, temporal and occipital lobes of the cortex; NTFs form in the locus coeruleus and in the allocortex of the medial temporal lobe. Both AP and tau pathologies spread through the brain during disease progression. Significant attention is now being paid to the mechanisms of pathological tau spread in AD with the goal of identifying targets for novel therapies to prevent disease progression. While AP pathology historically has been thought to be causative in AD, multiple clinicopathological evaluations as well as recent in vivo imaging studies argue that the cognitive status of AD patients correlates most closely with region-specific brain atrophy and distribution of the hyper-phosphorylated and aggregated pathological forms of tau that lead to the formation of NFTs. Longitudinal studies have confirmed that propagation of tau pathology correlates significantly with cognitive decline. These data suggest that suppression of propagation of tau pathology in AD may have a disease-modifying effect.

Disclosed herein are dual inhibitors of the enzymes neutral sphingomyelinase 2 (nSMase2) and acetylcholine esterase (AChE) a key enzyme implicated in AD. In in vitro studies, the disclosed inhibitors prevented tau spread in cell culture systems. A physiological level of nSMase2 activity is important for normal brain function, however, brain nSMase2 activity increases with age leading to dysregulation in sphingomyelin turnover. Specifically, there is over-activation of nSMase2 in AD and brain ceramide levels have been found to be elevated in AD patients compared to age-matched control subjects. AD-related ceramide/sphingomyelin imbalance is greater in individuals that express apolipoprotein E4 (ApoE4), the major genetic risk factor for sporadic, late onset AD.

NSMase2 is a key enzyme for biogenesis of brain exosomes through the Endosomal Sorting Complex Required for Transportation (ESCRT)-independent pathway. Exosomes, a type of extracellular vesicle (EV), are ceramide-enriched vesicles 40-150 nm in diameter generated by inward budding of the endosomal membrane; they are expelled from brain cells when multivesicular endosomes fuse with the plasma membrane. Brain exosomes are part of normal intercellular communication but a subset of these exosomes produced by the ESCRT-independent pathway have been shown to carry disease-propagating proteopathic seeds, such as tau oligomers, in AD. Tau oligomers have been found to be associated with exosomes in both cell culture medium and transgenic AD/tauopathy model brain tissue, as well as in AD patient plasma and CSF. Exosomes isolated from human AD CSF have been shown to carry p-tau seeds that are able to propagate in vitro in tau biosensor cells expressing Tau^{RDΔK}.

The current armamentarium of nSMase2 inhibitors is inadequate for development as potential preclinical lead candidates as they have poor drug-like properties and oral brain permeability. Thus, the goal was to identify an nSMase2 inhibitor that overcame these limitations. Using an nSMase2 inhibitor screening assay, a furoindoline compound was identified as a ‘validated hit’. Medicinal chemistry studies were conducted to identify a series of compounds that not only inhibit nSMase2 activity and p-tau seed propagation, but also inhibit acetylcholinesterase (AChE) were conducted.

AChE inhibitors (AChEIs) are currently one of only two classes of FDA-approved AD therapeutics; they have demonstrated treatment of AD, being most effective in mild and moderate AD. Inhibition of AChE leads to increased levels of acetylcholine (ACh) at the synapse and in brain parenchyma and provides support for cholinergic synaptic plasticity even during progressive loss of cholinergic innervation from the basal forebrain.

Interestingly, in mild to moderate AD, there is significantly decreased neuronal cholinergic activity and high levels of p-tau in CSF-derived exosomes, thus treatment of patients in these stages of the disease with dual nSMase2/AChE inhibitors has the potential to be highly beneficial.

The dual nSMase2/AChE inhibitors described herein represent a class of agents that are disease-modifying by attenuating and suppressing disease progression through inhibition of exosome-mediated tau propagation during the early and/or middle stages of AD while also providing symptomatic relief through support of ACh-mediated cognitive enhancement. The proposed mechanism of action of these dual inhibitors comprises two aspects: first, based on recent reports supporting a role for nSMase2 inhibition in suppression of release of tau oligomers in exosomes from presynaptic neurons, the dual inhibitor may attenuate propagation of disease. Second, AChE inhibition could lead to the suppression of uptake of the tau oligomers through ACh receptors, specifically the muscarinic M1 or M3 receptors on postsynaptic neurons by maintaining competition for these receptors through increased ACh levels at the synapse.

The discovery of a class of potent nSMase2/AChE dual inhibitors presents an opportunity for further development of these agents as a new therapeutic approach to Alzheimer's disease. The data supports the ability of the dual inhibitors to suppress tau propagation in vitro and release of exosome-bearing EVs in vivo in an AD model. The effects of the dual nSMase2/AChE inhibitors will include support of cholinergic synaptic plasticity, reduction of neuroinflammation (37), and most importantly suppression of exosome-mediated tau propagation and tau uptake through M1/M3 muscarinic ACh receptors. In concert, these mechanisms of action have the potential to not only address symptoms of AD by enhancing cholinergic activity but also to suppress cell-to-cell tau propagation significantly altering an underlying cause of AD and thus be truly disease-modifying.

In one aspect, the present disclosure provides compounds having a structure of Formula I:

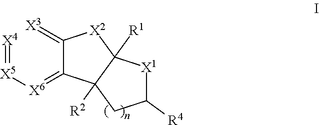

or a pharmaceutically acceptable salt thereof, wherein:
X¹ and X² are each independently O or NR^{a};
X³, X⁴, X⁵, and X⁶ are each independently CR³ or N;
R¹ is hydrogen or alkyl;
R² is alkyl, haloalkyl, alkoxy, cycloalkyl, aryl, aralkyl, or haloalkyl;
each R³ is independently selected from hydrogen, halogen, —OC(O)NR^{b}R^{c}, —NC(O)NR^{b}R^{c}, alkyl, haloalkyl, alkoxy, aralkyloxy, cycloalkyl, aryl, or heteroaryl;
R⁴ is hydrogen or alkyl;
R^{a} is hydrogen, alkyl, —C(O)-alkyl, —S(O)₂-alkyl, or —S(O)₂-aryl;
R⁶ is hydrogen, alkyl, cycloalkyl, aryl, heteroaryl, or heterocyclyl;
R^{c} is hydrogen or alkyl; and
n is an integer from 1 to 4.

In certain embodiments, the compound is not

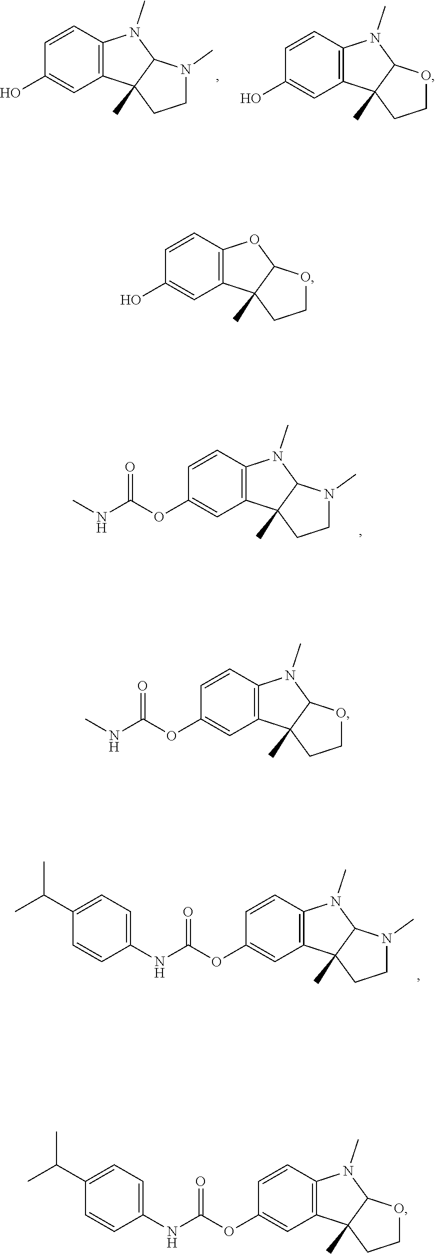

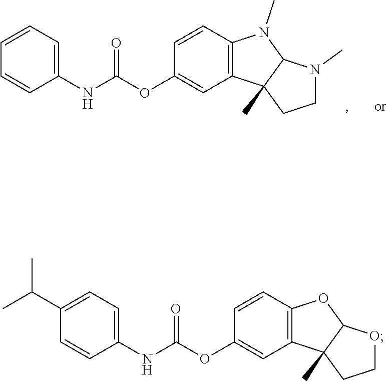

or a salt thereof.

In certain embodiments, the compound has a structure of Formula Ia or Ib:

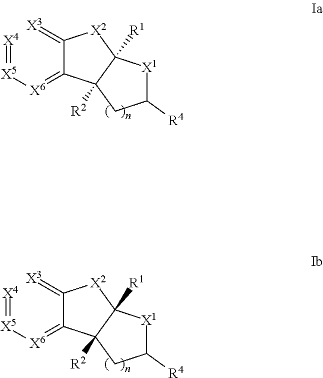

or a pharmaceutically acceptable salt thereof.

In certain embodiments, the compound has a structure of Formula IIa, IIb, or IIc:

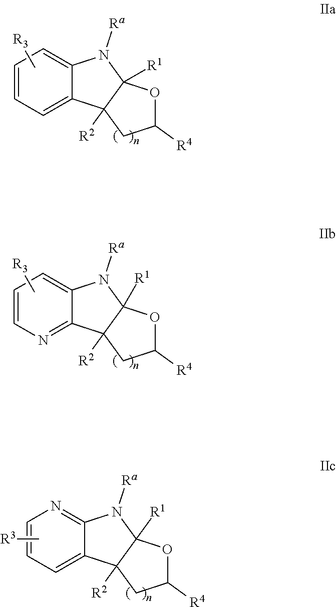

or a pharmaceutically acceptable salt thereof.

In certain preferred embodiments, the compound has a structure of Formula IIIa, IIIb, or IIIc:

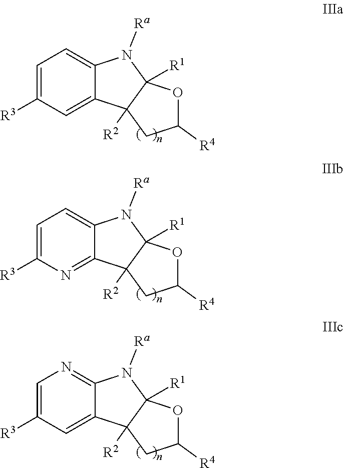

or a pharmaceutically acceptable salt thereof.

In certain embodiments, R¹ is hydrogen.

In certain preferred embodiments, R² is C₁-C₄-alkyl (e.g., methyl) or C₂-C₄-alkenyl (e.g., allyl).

In certain preferred embodiments, R³ is aralkyloxy (e.g., benzyloxy), bromo, chloro, aryl (e.g., methoxyphenyl), —OC(O)NR^{b}R^{c}, —NC(O)NR^{b}R^{c}, or C₁-C4-alkoxy (e.g., methyoxy).

In certain preferred embodiments, R^{a} is hydrogen. In other embodiments, at least one R^{a} is alkyl (e.g., methyl). In certain preferred embodiments, two R^{a} are alkyl (e.g., methyl).

In certain embodiments, the compound has a structure of Formula IVa, IVb, or IVc:

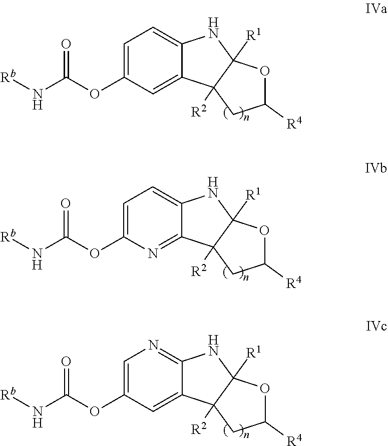

or a pharmaceutically acceptable salt thereof.

In certain embodiments, n is 1 or 2.

In certain embodiments R⁴ is hydrogen. In other embodiments, R⁴ is alkyl (e.g., ethyl).

In certain embodiments, the compound has a structure of Formula Va, Vb, or Vc:

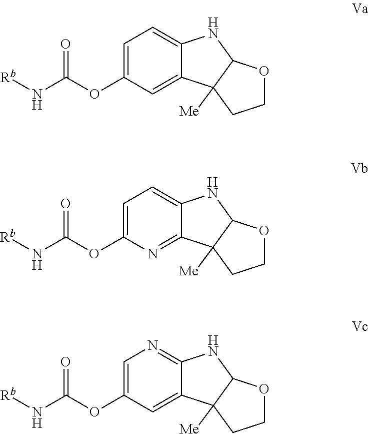

or a pharmaceutically acceptable salt thereof.

In some preferred embodiments, Rb is aryl (e.g., phenyl, methoxyphenyl, dimethoxyphenyl, trifluoromethyloxyphenyl, methylphenyl, dimethylphenyl), cycloalkyl (e.g., cyclohexyl), or heteroaryl (e.g., pyridyl, fluoropyridyl, methylpyridyl, pyrimidinyl, pyridazinyl, or pyrazinyl).

In certain embodiments, the compounds of the disclosure are dual inhibitors of nSMase2 and AChE.

In certain embodiments, the compound of formula I is selected from a compound recited in Table 1.

TABLE 1
Exemplary Compounds of the Present Invention

| | IC₅₀ (nSMase2) | AChE |
|---|---|---|
| Structure | Groups: A-D | activity |
| 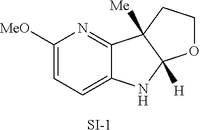 | D | |
| 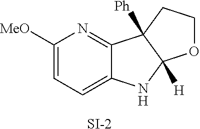 | D | |
| 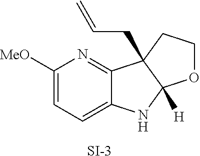 | D | |
| 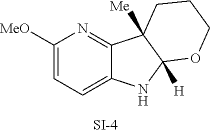 | D | |
| 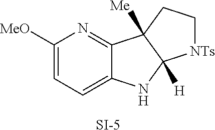 | C | |
| 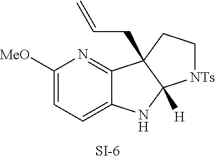 | D | |
| 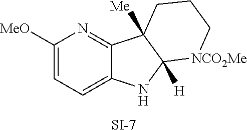 | D | |
| 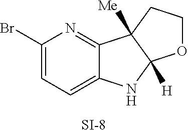 | D | |
| 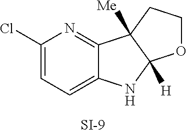 | D | |
| 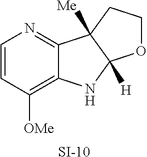 | D | |
| 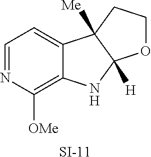 | D | |
| 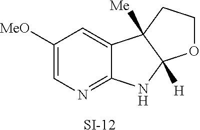 | D | |
| 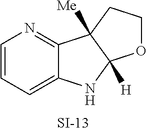 | D | |
| 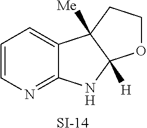 | D | |
| 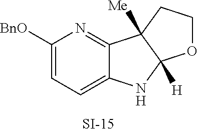 | D | |
| 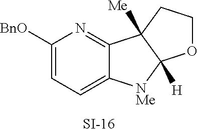 | D | |
| 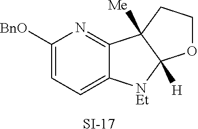 | D | |
| 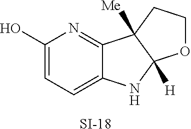 | D | |
| 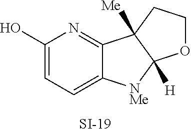 | B | |
| 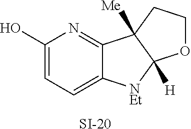 | B | |
| 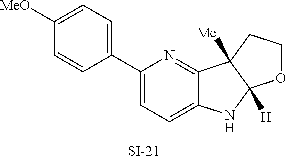 [SMILES: COc1ccc(-c2ccc3c(n2)[C@]2(C)CCO[C@@H]2N3)cc1 ; "SI-21"] | | |
| 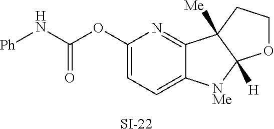 | B | |
| 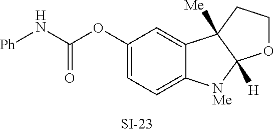 | B | A* |
| 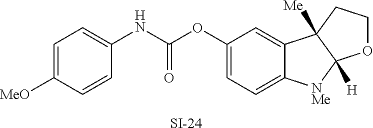 | B | C* |
| 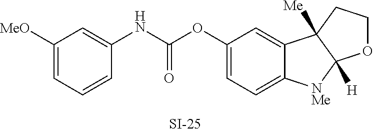 | B | A* |
| 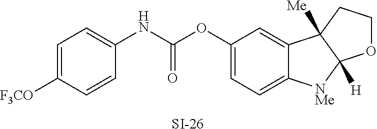 | C | D* |
| 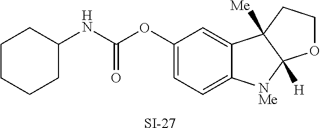 [SMILES: CN1c2ccc(OC(=O)NC3CCCCC3)cc2[C@]2(C)CCO[C@H]12 ; "SI-27"] | B | A* |
| 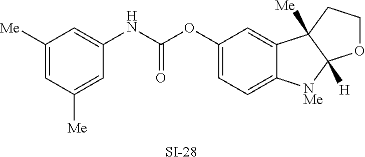 [SMILES: Cc1cc(C)cc(NC(=O)Oc2ccc3c(c2)[C@]2(C)CCO[C@@H]2N3C)c1 ; "SI-28"] | B | A* |
| 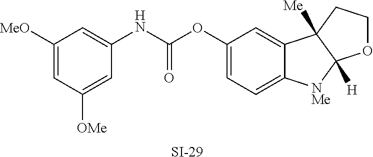 [SMILES: COc1cc(NC(=O)Oc2ccc3c(c2)[C@]2(C)CCO[C@@H]2N3C)cc(OC)c1 ; "SI-29"] | B | A* |
| 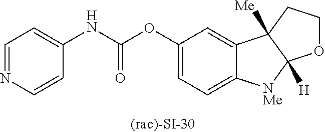 [SMILES: CN1c2ccc(OC(=O)Nc3ccncc3)cc2[C@]2(C)CCO[C@H]12 ; "(rac)-SI-30"] | A | D* |
| 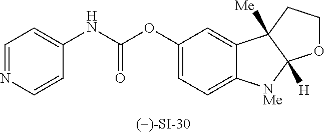 [SMILES: CN1c2ccc(OC(=O)Nc3ccncc3)cc2[C@]2(C)CCO[C@H]12 ; "(-)-SI-30"] | A | + |
| 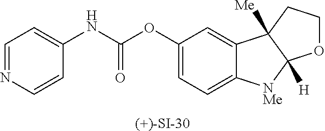 [SMILES: CN1c2ccc(OC(=O)Nc3ccncc3)cc2[C@]2(C)CCO[C@H]12 ; "(+)-SI-30"] | A | + |
| 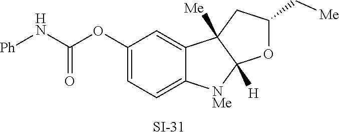 | B | C* |
| 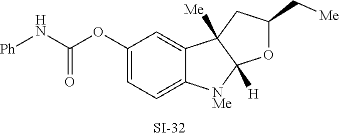 | B | C* |
| 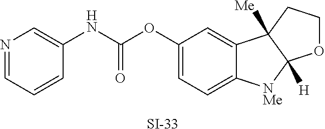 [SMILES: CN1c2ccc(OC(=O)Nc3cccnc3)cc2[C@]2(C)CCO[C@H]12 ; "SI-33"] | A | B |
| 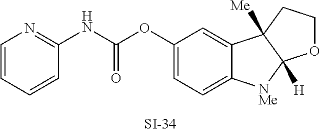 [SMILES: CN1c2ccc(OC(=O)Nc3ccccn3)cc2[C@]2(C)CCO[C@H]12 ; "SI-34"] | A | D* |
| 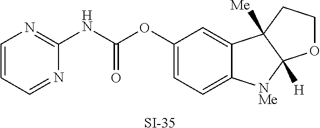 [SMILES: CN1c2ccc(OC(=O)Nc3ncccn3)cc2[C@]2(C)CCO[C@H]12 ; "SI-35"] | | |
| 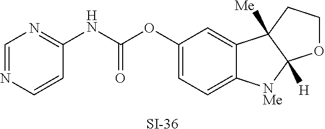 [SMILES: CN1c2ccc(OC(=O)Nc3ccncn3)cc2[C@]2(C)CCO[C@H]12 ; "SI-36"] | | |
| 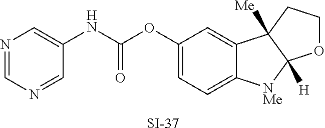 [SMILES: CN1c2ccc(OC(=O)Nc3cncnc3)cc2[C@]2(C)CCO[C@H]12 ; "SI-37"] | | |
| 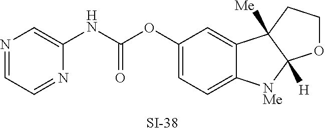 [SMILES: CN1c2ccc(OC(=O)Nc3cnccn3)cc2[C@]2(C)CCO[C@H]12 ; "SI-38"] | | |
| 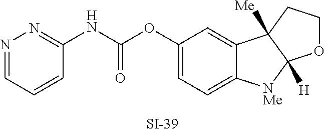 [SMILES: CN1c2ccc(OC(=O)Nc3cccnn3)cc2[C@]2(C)CCO[C@H]12 ; "SI-39"] | | |
| 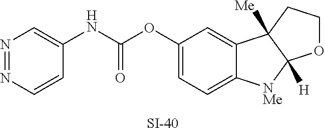 [SMILES: CN1c2ccc(OC(=O)Nc3ccnnc3)cc2[C@]2(C)CCO[C@H]12 ; "SI-40"] | | |
| 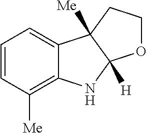 [SMILES: Cc1cccc2c1N[C@H]1OCC[C@@]21C] | | |
| 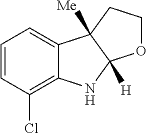 [SMILES: C[C@@]12CCO[C@@H]1Nc1c(Cl)cccc12] | | |
| 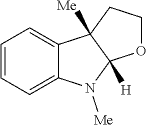 [SMILES: CN1c2ccccc2[C@]2(C)CCO[C@H]12] | | |
| 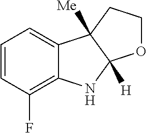 [SMILES: C[C@@]12CCO[C@@H]1Nc1c(F)cccc12] | | |
| 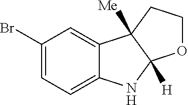 [SMILES: C[C@@]12CCO[C@@H]1Nc1ccc(Br)cc12] | | |
| 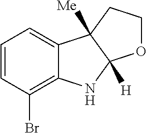 [SMILES: C[C@@]12CCO[C@@H]1Nc1c(Br)cccc12] | | |
| 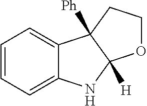 [SMILES: c1ccc([C@@]23CCO[C@@H]2Nc2ccccc23)cc1] | | |
| 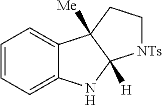 [SMILES: Cc1ccc(S(=O)(=O)N2CC[C@@]3(C)c4ccccc4N[C@@H]23)cc1] | | |
| 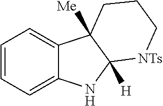 [SMILES: Cc1ccc(S(=O)(=O)N2CCC[C@@]3(C)c4ccccc4N[C@@H]23)cc1] | | |
| 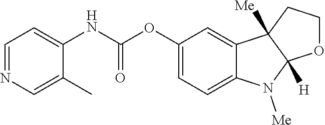 [SMILES: Cc1cnccc1NC(=O)Oc1ccc2c(c1)[C@]1(C)CCO[C@@H]1N2C] | | |
| 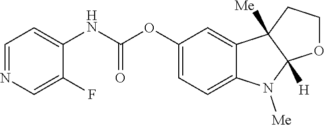 [SMILES: CN1c2ccc(OC(=O)Nc3ccncc3F)cc2[C@]2(C)CCO[C@H]12] | A | D* |
| 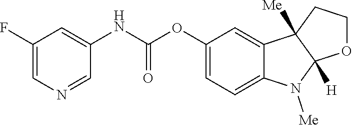 [SMILES: CN1c2ccc(OC(=O)Nc3cncc(F)c3)cc2[C@]2(C)CCO[C@H]12] | A | D* |
| 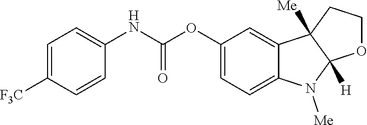 [SMILES: CN1c2ccc(OC(=O)Nc3ccc(C(F)(F)F)cc3)cc2[C@]2(C)CCO[C@H]12] | B | D* |
| 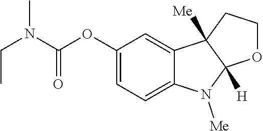 [SMILES: CCN(C)C(=O)Oc1ccc2c(c1)[C@]1(C)CCO[C@@H]1N2C] | B | D* |

A: < 1 μM;
B: 1-10 μM;
C: > 10 μM;
D: > 50 μM;
A*: < 0.5 μM;
B*: 0.5-2.5 μM;
C*: 2.5-5 μM;
D*: > 5 μM;
(+)-active as inhibitor in acetyl choline esterase assay (AChE).

In another aspect, the present disclosure provides methods of modulating neutral sphingomyelinase 2 (n-SMase2) in a cell, comprising contacting a cell with a compound disclosed herein. In certain embodiments, contacting the cell occurs in a subject suffering from a SMase2-mediated condition and/or disease.

In yet another aspect, the present disclosure provides methods of modulating acetylcholinesterase (AChE) in a cell, comprising contacting a cell with a compound disclosed herein. In certain embodiments, contacting the cell occurs in a subject suffering from a AChE-mediated condition and/or disease.

In yet another aspect, the present disclosure provides methods of modulating neutral sphingomyelinase 2 (n-SMase2) and acetylcholinesterase (AChE) in a cell, comprising contacting a cell with a compound disclosed herein. In certain embodiments, contacting the cell occurs in a subject suffering from a SMase2-mediated and AChE-mediated condition and/or disease.

In yet another aspect, the present disclosure provides methods of inhibiting the spread of Tau seeds from donor cells to recipient cells, comprising contacting the donor cells and/or the recipient with a compound disclosed herein. In certain embodiments, contacting the cells occur in a subject in need thereof.

In yet another aspect, the present disclosure provides methods of treating or preventing a neurodegenerative disease or condition, comprising administering to a subject in need thereof a compound disclosed herein. In certain embodiments, the neurodegenerative disease or condition is selected from a tauopathy, Alzheimer's disease, Parkinson's disease, Huntington's disease, Lewy body dementia, frontotemporal dementia, amyotrophic lateral sclerosis, multiple sclerosis, progressive supranuclear palsy, and age related cognitive decline. In certain embodiments, the neurodegenerative disease is Alzheimer's disease. In certain embodiments, the disease is age-related macular degeneration or glaucoma.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O—alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

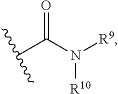

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

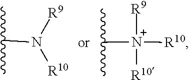

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

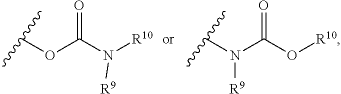

wherein R⁹ and R¹⁰ independently represent hydrogen or a hydrocarbyl group.

The term “carbocyclylalkyl”, as used herein, refers to an alkyl group substituted with a carbocycle group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “heteroalkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

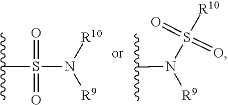

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

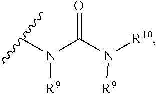

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Screening and Medicinal Chemical Optimization of Selective nSMase2 and Dual nSMase2/AChE Inhibitors

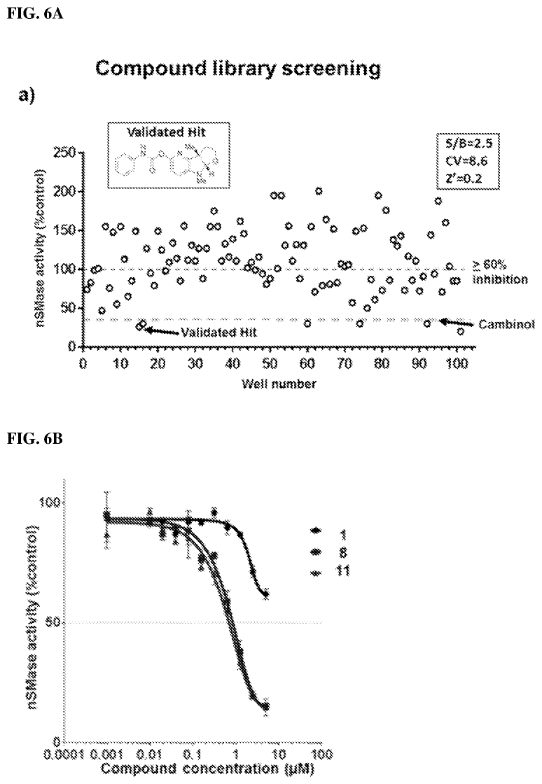

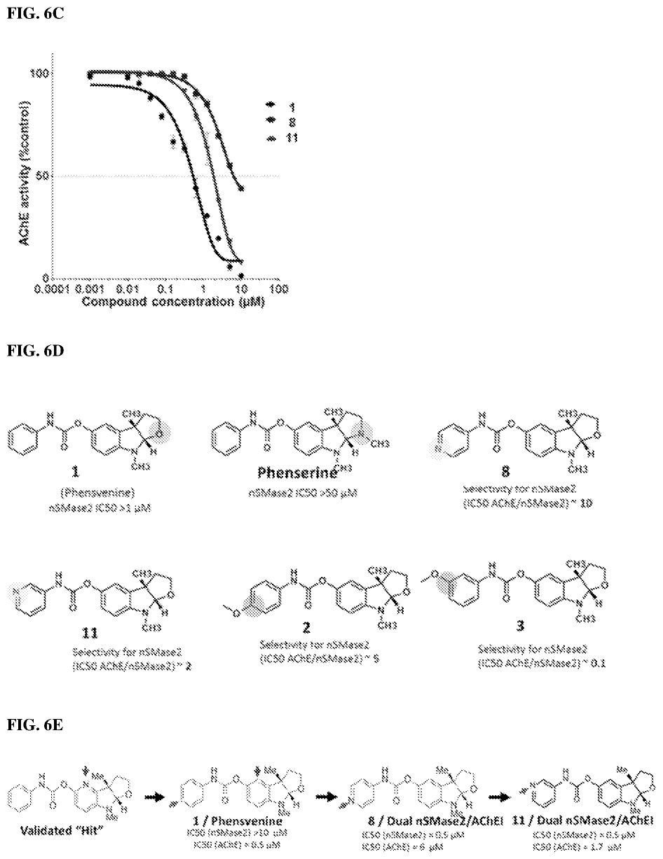

Screening of a small-molecule compound library using an Amplex Red Sphingomyelinase activity assay led to a number of hits that inhibited >60% of nSMase2 activity at a concentration of 50 μM, (FIG. 6A). After validation, one hit was selected for further medicinal chemistry optimization (FIG. 6A). Known nSMase2 inhibitor cambinol was used as a positive control in the screening assay.

Structural similarity between the validated hit and known AChE inhibitors phensvenine and phenserine prompted investigations to determine if those compounds are also nSMase2 inhibitors. Dose-response analysis revealed that phensvenine (1) indeed has nSMase2 inhibitory activity (FIG. 6B) but is a more potent inhibitor of AChE with an IC₅₀=0.5 μM (FIG. 6C). Interestingly, replacement of the oxygen in the furoindoline ring of phensevenine (O→N—CH3) yields phenserine, a commercially available AChE inhibitor and results in loss of nSMase2 inhibitory activity (IC₅₀>50 μM).

Compound 1 was then modified to synthesize a series of compounds with the goal of elucidating SAR information for dual nSMase2/AChE inhibition. These furoindoline compounds were synthesized using an interrupted Fisher indolization strategy; the general synthetic approach for compounds 1-7 is shown in Scheme 1 using substituted phenyl or cyclohexyl isocyanates.

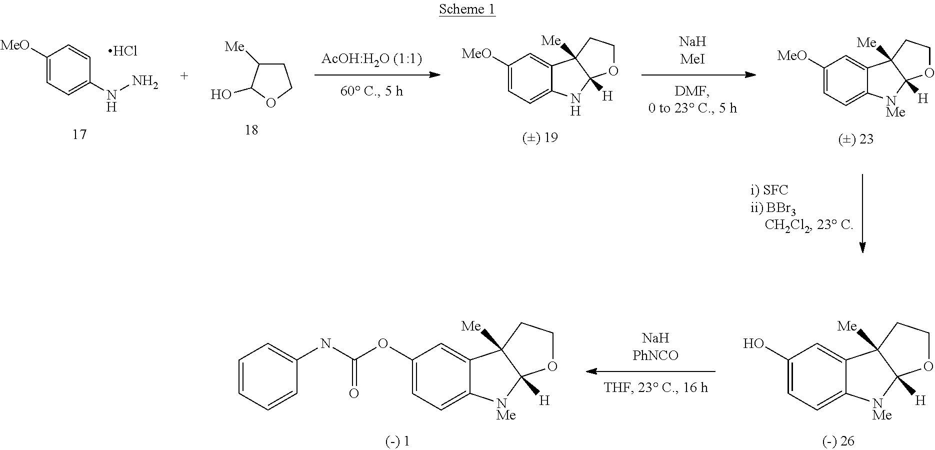

The synthetic approach of the key compounds 8 and 11 is shown in Scheme 2 starting from (−) 26 obtained after chiral separation. Compounds 13-16 were prepared according to Scheme 2 using the appropriate substituted anilines or N-ethylmethylamine

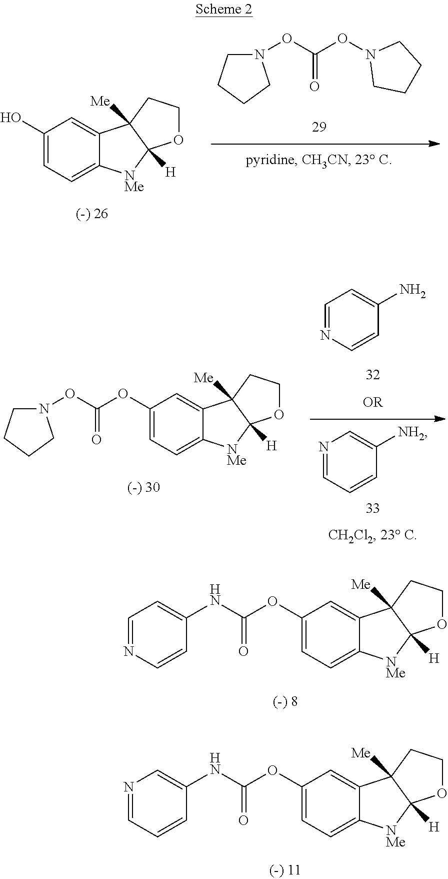

For compounds 9 and 10 we used the Scheme 3. Supercritical fluid chromatography (SFC) was used for chiral separation.

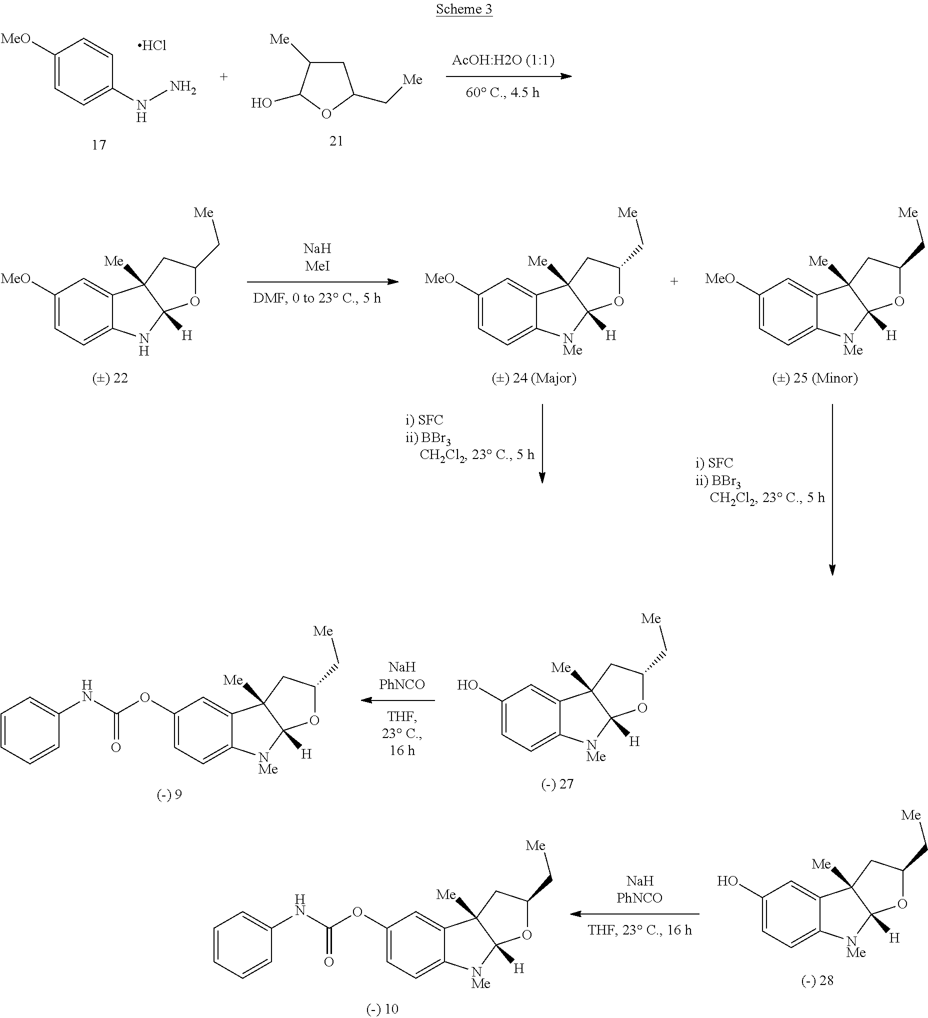

Sixteen compounds were prepared as part of the exploratory medicinal chemistry efforts and dose-response analysis was performed to obtain IC₅₀ values (Table 2).

The SAR analysis reveals a couple of key structural elements in this series required for enhanced nSMase2 and/or AChE inhibition (FIG. 6D). Substitutions in the carbamate phenyl ring pointed to a critical role for positions 3 and 4 as key control elements for nSMase2 and/or AChE inhibition. Substitution in the 4-position leads to increased selectivity for nSMase2 inhibition (such as for compounds 2, 4 and 8). In contrast, substitution in the 3-position leads to increased selectivity for AChE inhibition (such as 3 and 11). Introduction of electron donating groups (3, 6, and 7) at position 3 increased potency of AChE inhibition, while an electron withdrawing group (such as in 14) resulted in decreased potency. Importantly, replacement of the phenyl ring with a pyridyl ring in the carbamate moiety generally decreased potency of AChE inhibition and markedly enhanced potency for nSMase2 inhibition (e.g. 8, 11, 12). Most of the compounds (except 4) showed high predicted brain permeability by in silico StarDrop analysis and in a parallel artificial membrane permeability assay (PAMPA) (Table 2). A low degree of binding to human serum albumin (HSA) measured for most of the compounds, especially 8, 11, 12, and 13 (Table 2).

TABLE 2
Exemplary Compounds of the Present Invention

| Com- | | | | | | | HSA |
|---|---|---|---|---|---|---|---|
| pound | | | IC50 | IC50 | CNS perm | | (% un- |
| # | Structure | MW | nSMase2 | AChE | (PAMPA) | cLogP | bound) |
| 1 (Phens- venine) | 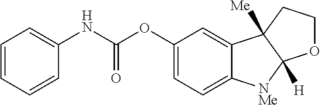 [SMILES: CN1c2ccc(OC(=O)Nc3ccccc3)cc2[C@]2(C)CCO[C@H]12] | 324.15 | >1 | 0.5 | 3.2 | 3.69 | 12 |
| 2 | 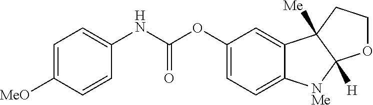 [SMILES: COc1ccc(NC(=O)Oc2ccc3c(c2)[C@]2(C)CCO[C@@H]2N3C)cc1] | 354.16 | 0.7 | 3.6 | 2 | 3.73 | 15 |
| 3 | 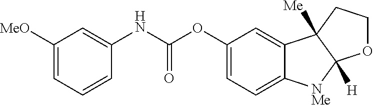 [SMILES: COc1cccc(NC(=O)Oc2ccc3c(c2)[C@]2(C)CCO[C@@H]2N3C)c1] | 354.16 | 1 | 0.1 | 2.3 | 3.73 | 10 |
| 4 | 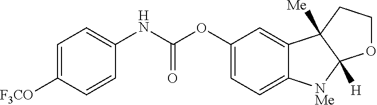 [SMILES: CN1c2ccc(OC(=O)Nc3ccc(OC(F)(F)F)cc3)cc2[C@]2(C)CCO[C@H]12] | 408.13 | 2.7 | >5 | 0.7 | 4.84 | 33 |
| 5 | 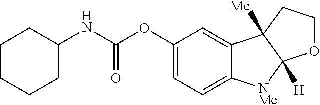 [SMILES: CN1c2ccc(OC(=O)NC3CCCCC3)cc2[C@]2(C)CCO[C@H]12] | 330.19 | 3.4 | 0.3 | 2.8 | 3.66 | 17 |
| 6 | 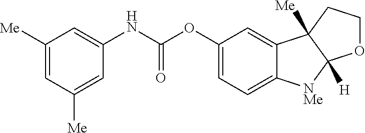 [SMILES: Cc1cc(C)cc(NC(=O)Oc2ccc3c(c2)[C@]2(C)CCO[C@@H]2N3C)c1] | 352.18 | 3.6 | 0.2 | 3.1 | 4.68 | 4 |
| 7 | 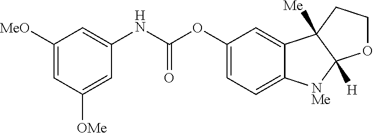 [SMILES: COc1cc(NC(=O)Oc2ccc3c(c2)[C@]2(C)CCO[C@@H]2N3C)cc(OC)c1] | 384.17 | 2 | 0.3 | 1.8 | 3.75 | 9 |
| 8 | 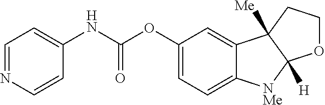 [SMILES: CN1c2ccc(OC(=O)Nc3ccncc3)cc2[C@]2(C)CCO[C@H]12] | 325.14 | 0.5 | 6 | 1.9 | 2.86 | 28 |
| 9 | 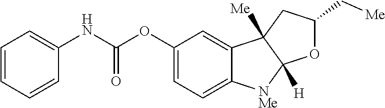 [SMILES: CC[C@H]1C[C@@]2(C)c3cc(OC(=O)Nc4ccccc4)ccc3N(C)[C@H]2O1] | 352.18 | >5 | 2.5 | 3 | 4.73 | 7 |
| 10 | 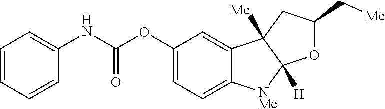 [SMILES: CC[C@@H]1C[C@@]2(C)c3cc(OC(=O)Nc4ccccc4)ccc3N(C)[C@H]2O1] | 352.18 | 5 | 3.7 | 3.2 | 4.73 | 5 |
| 11 | 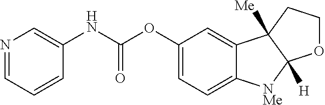 [SMILES: CN1c2ccc(OC(=O)Nc3cccnc3)cc2[C@]2(C)CCO[C@H]12] | 325.14 | 0.5 | 1.7 | 1.8 | 2.86 | 31 |
| 12 | 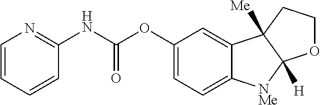 [SMILES: CN1c2ccc(OC(=O)Nc3ccccn3)cc2[C@]2(C)CCO[C@H]12] | 325.14 | 0.8 | >5 | 1.9 | 2.86 | 28 |
| 13 | 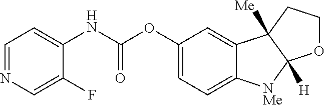 [SMILES: CN1c2ccc(OC(=O)Nc3ccncc3F)cc2[C@]2(C)CCO[C@H]12] | 343.13 | 0.6 | >5 | 1.3 | 2.65 | 35 |
| 14 | 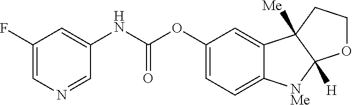 [SMILES: CN1c2ccc(OC(=O)Nc3cncc(F)c3)cc2[C@]2(C)CCO[C@H]12] | 343.13 | 0.9 | >5 | 2 | 3.1 | 21 |
| 15 | 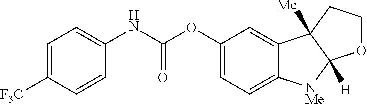 [SMILES: CN1c2ccc(OC(=O)Nc3ccc(C(F)(F)F)cc3)cc2[C@]2(C)CCO[C@H]12] | 392.13 | 5 | >5 | 2.2 | 4.94 | 7 |
| 16 | 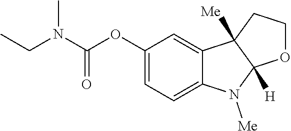 [SMILES: CCN(C)C(=O)Oc1ccc2c(c1)[C@]1(C)CCO[C@@H]1N2C] | 337.42 | >5 | >5 | NA | NA | NA |

Based on the SAR, the two dual inhibitors, 8 and 11, with 10- and 2-fold selectivity for nSMase2 inhibition over AChE, respectively, were further evaluated in in vitro and in vivo assays for exosomal tau release. Dual inhibition dose-response analyses for compounds 8 and 11 are shown in FIGS. 6b and 6C. The SAR from the medicinal chemistry efforts is summarized in FIG. 6E and is as follows: (1) replacement of ‘N’ in the furoindoline ring of the validated hit yields 1 (phensvenine) that is a dual inhibitor showing weak nSMase2 inhibition but potent AChE (1050=0.5 μM) inhibition; (2) the 4-pyridyl ring in the carbamate group leads to 8, a dual inhibitor with 10-fold selectivity for nSMase2 inhibition (IC₅₀=0.5 μM) over AChE inhibition; and (3) the 3-pyridyl carbamate compound 11 was a dual inhibitor with 2-fold selectivity for nSMase2 (IC₅₀=0.5 μM) and AChE (IC₅₀=1.7 μM) inhibition.

### Example 2: Preparation of Exemplary Compounds

### Materials and Methods.

Unless stated otherwise, reactions were conducted in flame-dried glassware under an atmosphere of air and commercially obtained reagents were used as received. Sodium hydride, boron tribromide, boron trichloride, phenyl isocyanate, N,N′-disuccinimidyl carbonate SI-13, cyclohexyl isocyanate, 3,5-dimethylphenyl isocyanate, 3,5-dimethoxyphenyl isocyanate, 3-aminopyridine SI-16, 4-aminopyridine SI-15, and 4-(trifluoromethyl)aniline SI-19 were obtained from Sigma-Aldrich. Hydrazine SI-1, 4-methoxyphenyl isocyanate, 3-methoxyphenyl isocyanate, and N-ethylmethylamine amine SI-20 were obtained from Oakwood Products, Inc. 4-(trifluoromethyl)phenyl isocyanate and 3-amino-5-fluoropyridine SI-18 were obtained from Combi-Blocks. Methyl iodide was obtained from Alfa Aesar. Reaction temperatures were controlled using an IKAmag temperature modulator, and unless stated otherwise, reactions were performed at room temperature (approximately 23° C.). Thin-layer chromatography (TLC) was conducted with EMD gel 60 F254 pre-coated plates (0.25 mm for analytical chromatography and 0.50 mm for preparative chromatography) and visualized using a combination of UV, anisaldehyde, iodine, and potassium permanganate staining techniques. Silicycle Siliaflash P60 (particle size 0.040-0.063 mm) was used for flash column chromatography. ¹H NMR spectra were recorded on Bruker spectrometers (500 MHz) and are reported relative to residual solvent signals. Data for ¹H NMR spectra are reported as follows: chemical shift (6 ppm), multiplicity, coupling constant (Hz), integration. Data for ¹³C NMR are reported in terms of chemical shift (125 MHz). ¹⁹F NMR spectra were recorded on Bruker spectrometers (at 376 MHz) and reported in terms of chemical shifts (δ ppm). Data for IR spectra were recorded on a Perkin-Elmer UATR Two FT-IR spectrometer and are reported in terms of frequency absorption (cm⁻¹). DART-MS spectra were collected on a Thermo Exactive Plus MSD (Thermo Scientific) equipped with an ID-CUBE ion source and a Vapur Interface (IonSense Inc.). Both the source and MSD were controlled by Excalibur software v. 3.0. The analyte was spotted onto OpenSpot sampling cards (IonSense Inc.) using volatile solvents (e.g. chloroform, dichloromethane). Ionization was accomplished using UHP He (Airgas Inc.) plasma with no additional ionization agents. The mass calibration was carried out using Pierce LTQ Velos ESI (+) and (−) Ion calibration solutions (Thermo Fisher Scientific). Optical rotations were measured with a Rudolph Autopol III Automatic Polarimeter. Any modification of the conditions shown in the representative procedures are specified in the corresponding schemes.

### Syntheses of Indoline Substrates

Representative Procedure for the synthesis of indoline substrates from Tables 1

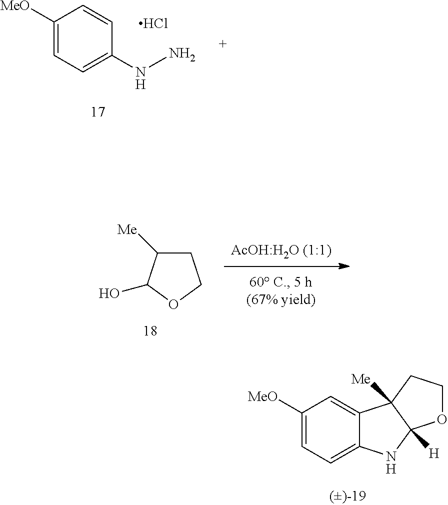 [SMILES: COc1ccc(NN)cc1 ; Cl ; CC1CCOC1O ; COc1ccc2c(c1)[C@]1(C)CCO[C@@H]1N2]

Indoline (±)-19. A 250 mL round-bottom flask containing a magnetic stir bar was charged with lactol 18 (3.37 g, 33.08 mmol, 1.0 equiv) followed by a solution of AcOH:H₂O (1:1, 170.0 mL, 0.2 M). Hydrazine 17 (5.78 g, 33.08 mmol, 1.0 equiv) was added and an air condenser attached to the flask. The reaction mixture was then placed into a pre-heated oil bath at 60° C. for 5 h. After the allotted time, the reaction mixture was taken out of the oil bath and allowed to cool to 23° C. over 20 min. The reaction mixture was then diluted with EtOAc (20 mL) and transferred to a separatory funnel. EtOAc (50 mL) and deionized water (50 mL) were then added, followed by a solution of saturated aqueous NaHCO₃ (450 mL). The layers were separated and the aqueous layer extracted with EtOAc (3×50 mL). The combined organics were washed with saturated aqueous NaCl (50 mL) and dried over MgSO₄. The volatiles were then removed under reduced pressure, and the crude residue was purified by flash column chromatography (3:1 Hexanes:EtOAc) to yield indoline ±19 (67% yield) as a red solid. Spectral data matches those previously reported.

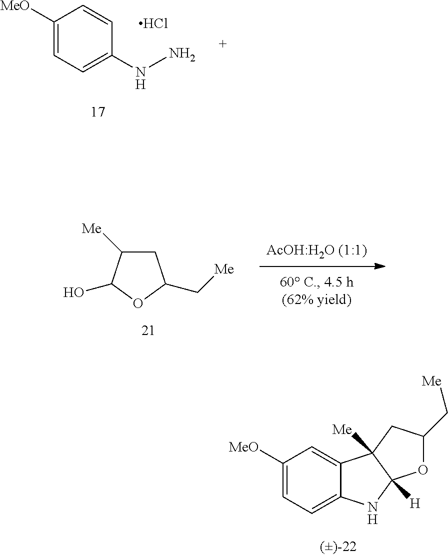 [SMILES: COc1ccc(NN)cc1 ; Cl ; CCC1CC(C)C(O)O1 ; CCC1C[C@@]2(C)c3cc(OC)ccc3N[C@H]2O1]

Indoline (±)-22. Purification by flash column chromatography (4:1 Hexanes:EtOAc) yielded indoline (±)-22(62% yield) as an amorphous solid in a 1.5:1 ratio of diastereomers.

### Methylation of Indoline Substrates

Representative Procedure for the methylation of indoline substrates. ((±)-SI-7 is used as an example).

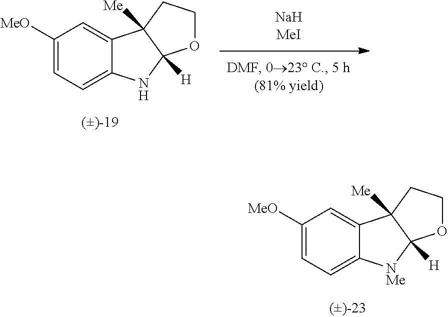 [SMILES: COc1ccc2c(c1)[C@]1(C)CCO[C@@H]1N2 ; COc1ccc2c(c1)[C@]1(C)CCO[C@@H]1N2C]

Indoline (±)-23. A 100 mL round-bottom flask was charged with a magnetic stir bar, flame-dried under reduced pressure, and allowed to cool under a N₂ atmosphere. Indoline (±)-SI-3 (4.52 g, 22.02 mmol, 1.0 equiv) was added and the flask was flushed with N₂ for 5 min. DMF (22.0 mL, 1.0 M) was added and the reaction mixture cooled to 0° C. for 10 min under an N2 atmosphere. NaH (60% dispersion in mineral oil, 1.90 g, 48.45 mmol, 2.2 equiv) was added in one portion and the reaction was left to stir for 30 min at 0° C. MeI (3.30 mL, 52.85 mmol, 2.4 equiv) was then added dropwise over 3 min After 30 min stirring at 0° C., the reaction mixture was warmed to 23° C. and allowed to stir for 4 h. The reaction mixture was then transferred to a separatory funnel with deionized H₂O (30 mL) and CH₂Cl₂ (30 mL), sequentially. The layers were separated and the aqueous layer was extracted with CH₂Cl₂ (3×50 mL). The combined organic layers were washed with deionized H₂O (3×50 mL), saturated aqueous NaCl (50 mL), and dried over Na₂SO₄. The volatiles were removed under reduced pressure, and the crude residue was purified by flash chromatography (5:1 Hexanes:EtOAc) to yield indoline (±)-SI-7 (3.92 g, 81% yield) as a colorless oil. Spectral data matches those previously reported. Chiral Preparative SFC: 21.2×250 mm Chiral Technologies AD-H SFC column, 7% i-PrOH, 40.0 mL/min, λ=210 nm, 40° C., nozzle pressure=100 bar CO₂, t_{R1}=3.2 min, [α]^{26.5}_{D}+58.67° (c=0.10, CH₂Cl₂); t_{R2}=5.1 min, [α]^{27.8}_{D}−40.00° (c=0.10, CH₂Cl₂).

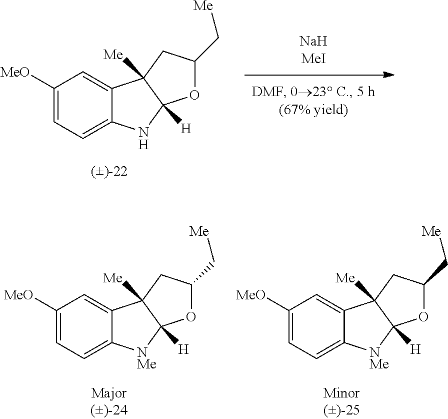 [SMILES: CCC1C[C@@]2(C)c3cc(OC)ccc3N[C@H]2O1 ; CC[C@H]1C[C@@]2(C)c3cc(OC)ccc3N(C)[C@H]2O1 ; CC[C@@H]1C[C@@]2(C)c3cc(OC)ccc3N(C)[C@H]2O1]

Indoline (±)-24 and (±)-25. Purification by preparative thin-layer chromatography (15:1 Hexanes:EtOAc) yielded indoline (±)-SI-8 and (±)-SI-9 (67% yield) in a 1.5:1 ratio of diastereomers as amorphous solids. Indoline (±)-SI-8 (major): R_{f} 0.50 (5:1 Hexanes:EtOAc); ¹H NMR (500 MHz, CDCl₃): δ 6.84-6.66 (m, 2H), 6.31 (d, J=8.3, 1H), 4.95 (s, 1H), 3.99 (app. p, J=6.74, 1H), 3.75 (s, 3H), 2.90 (s, 3H), 2.16 (dd, J=6.5, 12.3, 1H), 1.87 (dd, J=6.5, 12.3, 1H), 1.43-1.35 (m, 4H), 1.27-1.18 (m, 1H), 0.82 (t, J=7.3, 3H); ¹³C NMR (125 MHz, CDCl₃): 152.9, 143.7, 138.0, 112.1, 110.4, 107.0, 106.5, 80.3, 56.2, 52.4, 45.6, 32.5, 28.8, 24.4, 10.7; IR (film): 2958, 2935, 1596, 1497, 1280 cm⁻¹; HRMS-APCI (m/z) [M+H]⁺ calcd for C₁₅H₂₂NO₂, 248.16451. found 248.16371. Chiral Preparative SFC: 21.2×250 mm Chiral Technologies AD-H SFC column, 4% i-PrOH, 40.0 mL/min, 2=210 nm, 40° C., nozzle pressure=100 bar CO₂, t_{R1}=2.4 min, [α]^{25.3}_{D}+62.00° (c=0.10, CH₂Cl₂); t_{R2}=3.4 min. [α]^{26.7}_{D}−96.00° (c=0.10, CH₂Cl₂). Indoline (±)-SI-9 (minor): R_{f} 0.55 (5:1 Hexanes:EtOAc); ¹H NMR (500 MHz, CDCl₃): δ 6.68-6.65 (m, 2H), 6.28 (d, J=8.4, 1H), 5.05 (s, 1H), 3.76 (s, 3H), 3.59 (m, 1H), 2.88 (s, 3H), 2.19 (dd, J=4.4, 11.9, 1H), 1.70-1.58 (m, 2H), 1.50-1.43 (m, 4H), 0.86 (t, J=7.5, 3H); ¹³C NMR (125 MHz, CDCl₃): 152.7, 145.3, 136.7, 112.1, 110.6, 105.3, 105.2, 80.4, 56.2, 53.0, 47.1, 31.7, 27.8, 25.1, 10.4; IR (film): 2958, 2923, 1596, 1498, 1279 cm⁻¹; HRMS-APCI (m/z) [M+H]⁺ calcd for C₁₅H₂₂NO₂, 248.16451. found 248.16385. Chiral Preparative SFC: 21.2×250 mm Chiral Technologies AD-H SFC column, 4% i-PrOH, 40.0 ml/min, 2=210 nm, 40° C., nozzle pressure=100 bar CO₂, t_{R1}=2.1 min, [α]^{27.5}_{D}+105.33° (c=0.10, CH₂Cl₂); t_{R2}=2.9 min. [α]^{28.0}_{D}−91.33° (c=0.10, CH₂Cl₂).

The configuration of (±)-24 and (±)-25 was verified by 2_{D}-NOESY (500 MHz, CDCl₃), as the following correlations were observed:

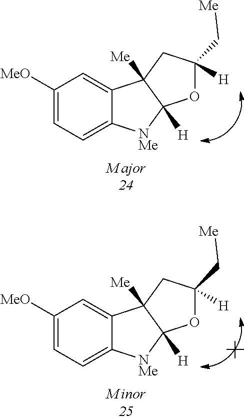 [SMILES: CC[C@H]1C[C@@]2(C)c3cc(OC)ccc3N(C)[C@H]2O1 ; CC[C@@H]1C[C@@]2(C)c3cc(OC)ccc3N(C)[C@H]2O1]

### Removal of Protecting Groups

Representative Procedure A for the deprotection of substrates (−)-26, (±)-27, and (±)-28 from Tables 1. ((−)-26 is used as an example).

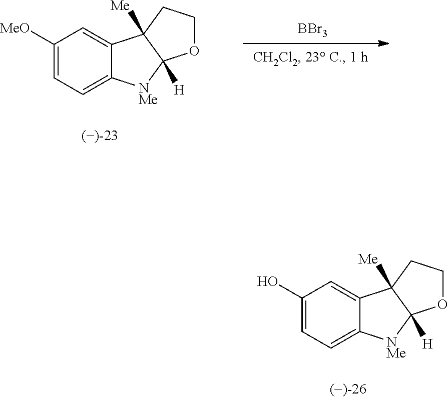 [SMILES: COc1ccc2c(c1)[C@]1(C)CCO[C@@H]1N2C ; "BBr3" ; CN1c2ccc(O)cc2[C@]2(C)CCO[C@H]12]

Indoline (−)-26 A 1-dram vial was charged with a magnetic stir bar, flame-dried under reduced pressure, and allowed to cool under a N₂ atmosphere. Indoline (−)-23 (15.0 mg, 0.068 mmol, 1.0 equiv) was added and the vial was flushed with N₂ for 5 min. CH₂Cl₂ (860 uL, 0.08 M) was added and the reaction mixture was left to run at 23° C. BBr₃ (1.0 M in CH₂Cl₂, 340.0 uL, 0.340 mmol, 5.0 equiv) was added dropwise over 1 min and the reaction was left to run at 23° C. for 1 h. After the allotted time, the volatiles were removed under N₂. MeOH (2.0 mL) was then added to the vial and the reaction mixture was allowed to stir for 5 min. The reaction was then concentrated under reduced pressure and the resulting residue was suspended in deionized water (3 mL). The suspension was then transferred to a separatory funnel with EtOAc (2 mL). A saturated aqueous solution of NaHCO₃ (5 mL) was added and the layers separated. The aqueous layer was then extracted with EtOAc (3×5 mL). The combined organic layers were washed with saturated aqueous NaCl (5 mL) and dried over Na₂SO₄. The volatiles were then removed under reduced pressure, and the crude residue was taken forward to the next step.

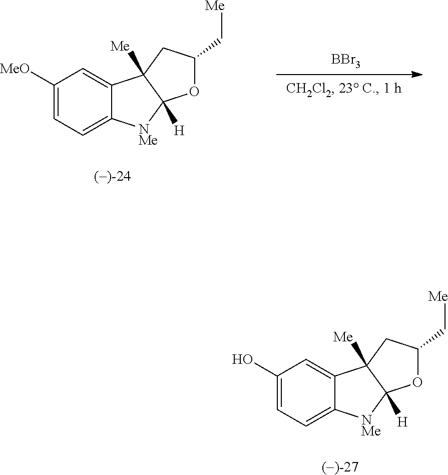 [SMILES: CC[C@H]1C[C@@]2(C)c3cc(OC)ccc3N(C)[C@H]2O1 ; "BBr3" ; CC[C@H]1C[C@@]2(C)c3cc(O)ccc3N(C)[C@H]2O1]

Indoline (−)-27. Following representative procedure A yielded indoline (−)-SI-11. The crude residue was taken forward to the next step.

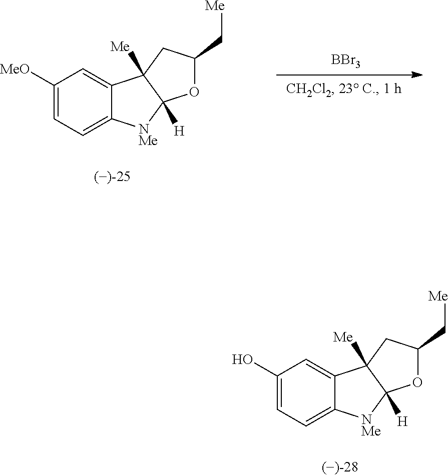 [SMILES: CC[C@@H]1C[C@@]2(C)c3cc(OC)ccc3N(C)[C@H]2O1 ; "BBr3" ; CC[C@@H]1C[C@@]2(C)c3cc(O)ccc3N(C)[C@H]2O1]

Indoline (−)-28. Following representative procedure A yielded indoline (−)-28. The crude residue was taken forward to the next step.

### Carbamoylation

Representative Procedure A for the synthesis of carbamates from Tables 1 ((−)-1 is used as an example).

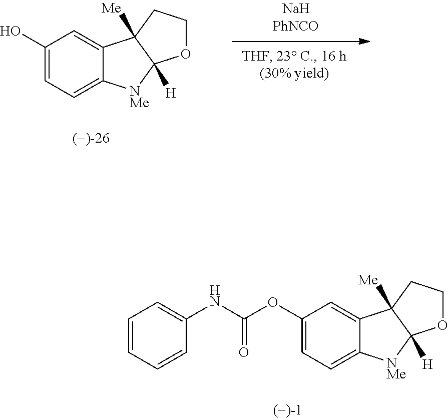 [SMILES: CN1c2ccc(O)cc2[C@]2(C)CCO[C@H]12 ; CN1c2ccc(OC(=O)Nc3ccccc3)cc2[C@]2(C)CCO[C@H]12]

Carbamate (−)-1. A 1-dram vial was charged with a magnetic stir bar, flame-dried under reduced pressure, and allowed to cool under a N₂ atmosphere. Indoline (−)-26 (10.0 mg, 0.049 mmol, 1.0 equiv) was added and the vial was flushed with N₂ for 5 min. The material was then dissolved in THF (244 uL, 0.2 M) followed by the addition of NaH (60% dispersion in mineral oil, 1.0 mg, 0.024 mmol, 0.5 equiv) in one portion under a constant flow of N2. PhNCO (6.9 mg, 6.4 uL, 0.058 mmol, 1.2 equiv) was then added dropwise over 1 min and the reaction was left to run at 23° C. for 16. After the allotted time, the reaction was quenched by addition of a saturated aqueous solution of NaHCO₃ (5 mL) and transferred to a separatory funnel with EtOAc (5 mL). The layers were separated and the aqueous layer was then extracted with EtOAc (3×5 mL). The combined organic layers were washed with saturated aqueous NaCl (5 mL) and dried over Na₂SO₄. The volatiles were then removed under reduced pressure, and the crude residue was purified by preparative thin-layer chromatography (1:1 Hexanes:EtOAc, 2% Et₃N) to yield carbamate (−)-1 (9.2 mg, 30% yield) as a brown solid. Carbamate (−)-1: R_{f} 0.45 (1:1 Hexanes:EtOAc); [α]^{31.0}_{D}−39.34° (c=0.10, CH₂Cl₂). Spectral data match those previously reported.

Representative Procedure B for the synthesis of carbamates from Tables 1. ((−)-12 is used as an example).

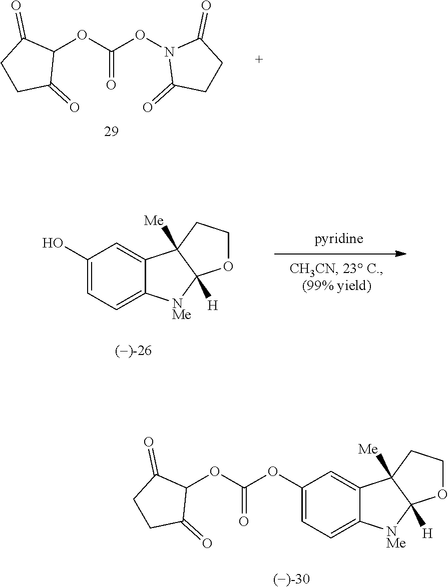 [SMILES: O=C(OC1C(=O)CCC1=O)ON1C(=O)CCC1=O ; CN1c2ccc(O)cc2[C@]2(C)CCO[C@H]12 ; CN1c2ccc(OC(=O)OC3C(=O)CCC3=O)cc2[C@]2(C)CCO[C@H]12]

Carbonate (−)-30. A 1-dram vial was charged with a magnetic stir bar, flame-dried under reduced pressure, and allowed to cool under a N₂ atmosphere. Indoline (−)-26 (75.0 mg, 0.370 mmol, 1.0 equiv) was added and the vial was flushed with N₂ for 5 min. The material was then dissolved in CH₃CN (730.0 uL, 0.50 M) and stirring began at 23° C. Pyridine (59.0 uL, 0.730 mmol, 2.0 equiv) was added in one portion under a constant flow of N2 followed by carbonate 29 (230.0 mg, 0.910 mmol, 2.5 equiv) in one portion. The reaction was then left to run at 23° C. for 18 h. After the allotted time, the reaction was diluted with CH₂Cl₂ (1.0 mL) and the solid that precipitated was collected by vacuum filtration over filter paper. The solid precipitate was then rinsed with CH₂Cl₂ (3 mL). The filtrate was concentrated under reduced pressure and the residue was suspended in EtOAc (5 mL) and transferred to a separatory funnel. The organic layer was washed with 5% aqueous citric acid (2×3 mL), saturated aqueous NaCl (5 mL), and dried over Na₂SO₄, sequentially. The volatiles were then removed under reduced pressure to yield carbonate (−)-30 (125.3 mg, 99% yield) as a white foam. Carbonate (−)-30: mp: 46-49° C.; R_{f} 0.38 (1:1 Hexanes:EtOAc); ¹H NMR (500 MHz, CDCl₃): δ 6.96 (dd, J=2.3, 8.3, 1H), 6.94 (d, J=2.3, 1H), 6.28 (d, J=8.3, 1H), 5.09 (s, 1H), 3.96 (ddd, J=1.6, 7.2, 8.7, 1H), 3.46 (ddd, J=5.2, 8.7, 11.1, 1H), 2.90 (s, 3H), 2.86 (s, 4H), 2.11 (ddd, J=1.6, 5.2, 12.1, 1H), 2.04 (ddd, J=7.2, 11.1, 12.1, 1H), 1.45 (s, 3H); ¹³C NMR (125 MHz, CDCl₃): 168.7, 151.0, 149.2, 149.2, 142.9, 135.9, 119.8, 115.3, 105.3, 104.5, 67.4, 52.5, 41.7, 31.1, 25.6, 24.7; IR (film): 2942, 2868, 1818, 1741, 1215 cm⁻¹; HRMS-APCI (m/z) [M+H]⁺ calcd for C₁₇H₁₉N₂O₆, 347.12376. found 347.12356; [α]^{31.0}_{D}−52.01° (c=0.10, CH₂Cl₂).

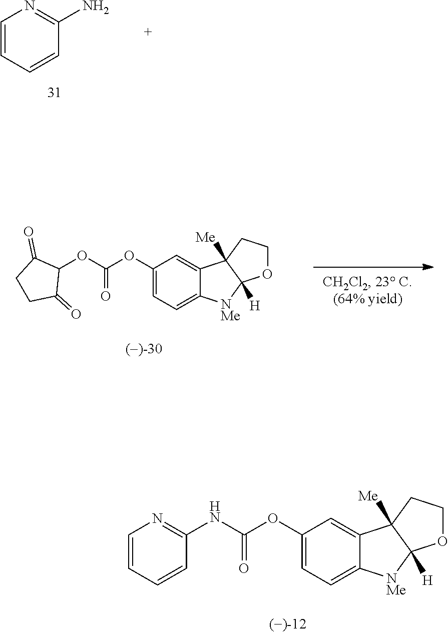 [SMILES: Nc1ccccn1 ; CN1c2ccc(OC(=O)OC3C(=O)CCC3=O)cc2[C@]2(C)CCO[C@H]12 ; CN1c2ccc(OC(=O)Nc3ccccn3)cc2[C@]2(C)CCO[C@H]12]

Carbamate (−)-12. A 1-dram vial was charged with a magnetic stir bar, flame-dried under reduced pressure, and allowed to cool under a N₂ atmosphere. Carbonate (−)-14 (10.0 mg, 0.029 mmol, 1.0 equiv) was added and the vial was flushed with N₂ for 5 min. The material was then dissolved in CH₂Cl₂ (600 uL, 0.05 M) and stirring began at 23° C. Amine SI-15 (5.4 mg, 0.057 mmol, 2.0 equiv) was then added in one portion under a constant flow of N2. The reaction was then left to run at 23° C. for 18 h. After the allotted time, the volatiles were removed under reduced pressure and the crude residue was purified by preparative thin-layer chromatography (1:1 Hexanes:EtOAc) to yield carbamate (−)-12 (6.0 mg, 64% yield) as an amorphous solid. Carbamate (−)-12: R_{f} 0.63 (1:5 Hexanes:EtOAc); ¹H NMR (500 MHz, CDCl₃): δ 8.33-8.32 (m, 1H), 8.30 (br. s, 1H), 8.00 (d, J=8.4, 1H), 7.72-7.68 (m, 1H), 7.02 (ddd, J=1.0, 4.9, 7.4, 1H), 6.92-6.89 (m, 2H), 6.32 (d, J=8.3, 1H), 5.09 (s, 1H), 3.96 (ddd, J=1.6, 7.3, 8.8, 1H), 3.50 (ddd, J=5.3, 8.8, 11.2, 1H), 2.92 (s, 3H), 2.14 (ddd, J=1.3, 5.3, 11.9, 1H), 2.08-2.02 (m, 1H), 1.47 (s, 3H); ¹³C NMR (125 MHz, CDCl₃): 152.6, 151.6, 148.6, 148.1, 142.2, 138.6, 135.6, 121.0, 119.3, 116.7, 112.6, 105.6, 104.8, 67.5, 52.5, 41.8, 31.3, 24.8; IR (film): 3180, 2955, 2925, 1746, 1588 cm⁻¹; HRMS-APCI (m/z) [M+H]⁺ calcd for C₁₈H₂₀N₃O₃, 326.14992. found 326.15099; [α]^{26.8}_{D} −102.7° (c=0.10, CH₂Cl₂).

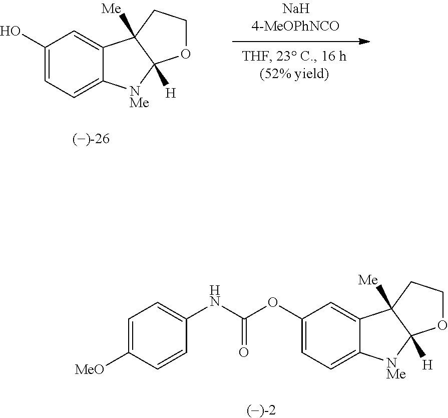 [SMILES: CN1c2ccc(O)cc2[C@]2(C)CCO[C@H]12 ; COc1ccc(NC(=O)Oc2ccc3c(c2)[C@]2(C)CCO[C@@H]2N3C)cc1]

Carbamate (−)-2. Following representative procedure A yielded carbamate (−)-2 (13.4 mg, 52% yield) as a white solid. Carbamate (−)-2: mp: 140-143° C.; R_{f} 0.56 (1:1 Hexanes:EtOAc); ¹H NMR (500 MHz, CDCl₃): δ 7.38-7.31 (m, 2H), 6.90-6.86 (m, 3H), 6.86-6.83 (m, 1H), 6.32-6.27 (m, 1H), 5.08 (s, 1H), 3.95 (ddd, J=1.5, 7.3, 8.7, 1H), 3.79 (s, 3H), 3.45 (ddd, J=5.2, 8.7, 11.1, 1H), 2.90 (s, 3H), 2.13 (ddd, J=1.4, 5.2, 11.9, 1H), 2.02 (ddd, J=7.2, 11.3, 11.3, 1H), 1.45 (s, 3H); ¹³C NMR (125 MHz, CDCl₃): δ 156.1, 152.8, 148.2, 142.4, 135.3, 130.7, 120.8, 120.6, 116.6, 114.3, 105.4, 104.7, 67.3, 55.5, 52.3, 41.6, 31.2, 24.6; IR (film): 3311, 2958, 1717, 1512, 1196 cm⁻¹; HRMS-APCI (m/z) [M+H]⁺ calcd for C₂₀H₂₃N₂O₄⁺, 355.16523. found, 355.16422; [α]^{30.7} _{D}−62.65° (c=0.10, CH₂Cl₂).

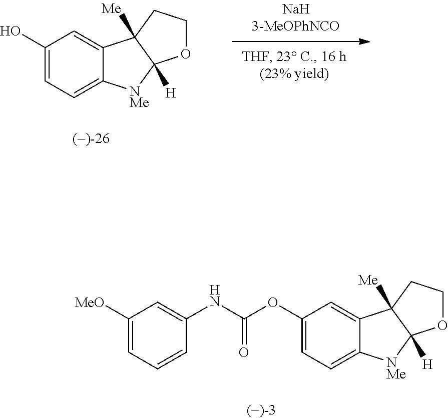 [SMILES: CN1c2ccc(O)cc2[C@]2(C)CCO[C@H]12 ; COc1cccc(NC(=O)Oc2ccc3c(c2)[C@]2(C)CCO[C@@H]2N3C)c1]

Carbamate (−)-3. Following representative procedure A yielded carbamate (−)-3 (12.0 mg, 23% yield) as a colorless oil. Carbamate (−)-3: R_{f} 0.65 (1:1 Hexanes:EtOAc); ¹H NMR (500 MHz, CDCl₃): δ 7.21 (app. t, J=8.2, 2H), 6.92-6.85 (m, 4H), 6.67-6.63 (m, 1H), 6.31 (d, J=4.2, 1H), 5.09 (s, 1H), 3.96 (ddd, J=1.5, 7.3, 8.7, 1H), 3.80 (s, 3H), 3.50 (ddd, J=5.2, 8.7, 11.1, 1H), 2.90 (s, 3H), 2.14 (ddd, J=1.4, 5.2, 11.9, 1H), 2.03 (ddd, J=7.2, 11.3, 11.3, 1H), 1.46 (s, 3H); ¹³C NMR (125 MHz, CDCl₃): δ 160.4, 152.5, 148.3, 142.2, 138.9, 135.4, 129.8, 120.9, 116.5, 110.8, 109.7, 105.4, 104.7, 104.2, 67.3, 55.3, 52.3, 41.6, 31.2, 24.6; IR (film): 3301, 2928, 1746, 1611, 1494, 1196 cm⁻¹; HRMS-APCI (m/z) [M+H]⁺ calcd for C₂₀H₂₃N₂O₄⁺, 355.16523. found, 355.16429; [α]^{25.2}_{D} −32.00° (c=0.10, CH₂Cl₂).

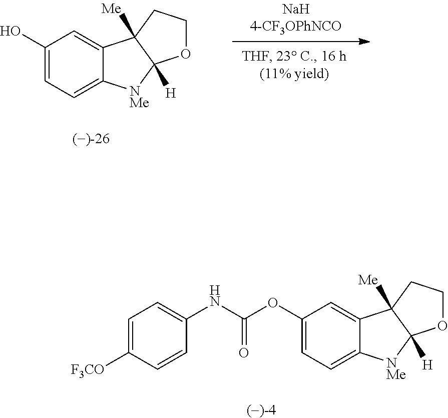 [SMILES: CN1c2ccc(O)cc2[C@]2(C)CCO[C@H]12 ; CN1c2ccc(OC(=O)Nc3ccc(OC(F)(F)F)cc3)cc2[C@]2(C)CCO[C@H]12]

Carbamate (−)-4. Following representative procedure A yielded carbamate (−)-4 (3.2 mg, 11% yield) as a white solid. Carbamate (−)-4: mp: 138-140° C.;

R_{f} 0.69 (1:1 Hexanes:EtOAc); ¹H NMR (500 MHz, CDCl₃): δ 7.47 (d, J=4.2, 2H), 7.19 (d, J=4.0, 2H), 6.90-6.85 (m, 2H), 6.31 (d, J=4.3, 1H), 5.09 (s, 1H), 3.96 (ddd, J=1.5, 7.3, 8.7, 1H), 3.50 (ddd, J=5.2, 8.7, 11.1, 1H), 2.91 (s, 3H), 2.13 (ddd, J=1.4, 5.2, 11.9, 1H), 2.04 (ddd, J=7.2, 11.3, 11.3, 1H), 1.46 (s, 3H); ¹³C NMR (125 MHz, CDCl₃): δ 152.5, 145.0, 142.1, 136.3, 135.5, 122.0, 120.8, 120.5 (q, J=293), 119.7, 116.4, 105.4, 104.6, 67.3, 52.3, 41.6, 31.1, 24.6; ¹⁹F-NMR (376 Hz, CDCl₃): δ−58.2; IR (film): 3322, 2929, 1719, 1549, 1497 cm⁻¹; HRMS-APCI (m/z) [M+H]⁺ calcd for C₂₀H₂₀F₃N₂O₄⁺, 409.1397. found, 409.13604; [α]^{30.2}_{D} −35.35° (c=0.10, CH₂Cl₂).

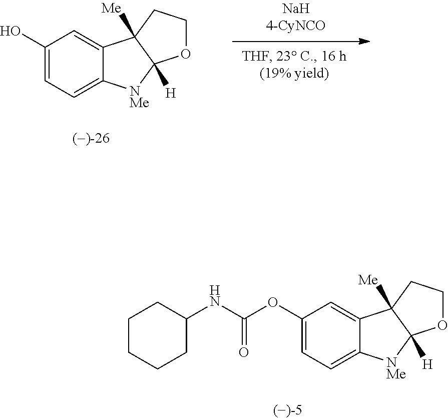 [SMILES: CN1c2ccc(O)cc2[C@]2(C)CCO[C@H]12 ; CN1c2ccc(OC(=O)NC3CCCCC3)cc2[C@]2(C)CCO[C@H]12]

Carbamate (−)-5. Following representative procedure A yielded carbamate (−)-5 (5.6 mg, 19% yield) as a white solid. Carbamate (−)-5: mp: 130-133° C.;

R_{f} 0.61 (1:1 Hexanes:EtOAc); ¹H NMR (500 MHz, CDCl₃): δ 6.83-6.80 (m, 2H), 6.27 (d, J=4.0, 1H), 5.06 (s, 1H), 4.83-4.82 (m, 1H), 3.94 (ddd, J=1.5, 7.3, 8.7, 1H), 3.57-3.54 (m, 1H), 3.48 (ddd, J=5.2, 8.7, 11.1, 1H), 2.89 (s, 3H), 2.12 (ddd, J=1.4, 5.2, 11.9, 1H), 2.05-1.99 (m, 3H), 1.74-1.71 (m, 2H), 1.64-1.60 (m, 1H), 1.44 (s, 3H), 1.41-1.32 (m, 2H), 1.25-1.17 (m, 3H); ¹³C NMR (125 MHz, CDCl₃): δ 154.6, 147.9, 142.9, 135.2, 120.7, 116.6, 105.5, 104.7, 67.3, 52.3, 50.1, 41.6, 33.3, 31.2, 25.5, 24.8, 24.6; IR (film): 3314, 2928, 1712, 1492.51, 1198 cm⁻¹; HRMS-APCI (m/z) [M+H]⁺ calcd for C₁₉H₂₇N₂O₃⁺, 331.20162. found, 331.20025; [α]^{28.7} _{D} −130.69° (c=0.10, CH₂Cl₂).

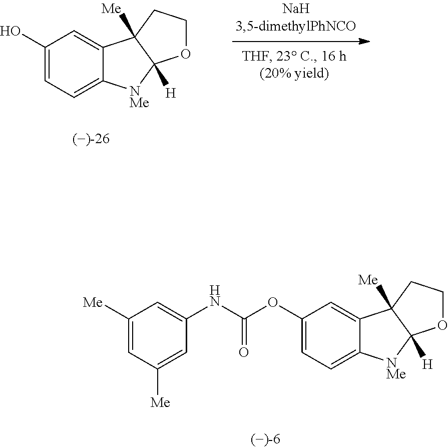 [SMILES: CN1c2ccc(O)cc2[C@]2(C)CCO[C@H]12 ; Cc1cc(C)cc(NC(=O)Oc2ccc3c(c2)[C@]2(C)CCO[C@@H]2N3C)c1]

Carbamate (−)-6. Following representative procedure A yielded carbamate (−)-6 (10.2 mg, 20% yield) as an amorphous solid. Carbamate (−)-6:

R_{f} 0.68 (1:1 Hexanes:EtOAc); ¹H NMR (500 MHz, CDCl₃): δ 7.07 (s, 2H), 6.90-6.85 (m, 2H), 6.73 (s, 1H), 6.33-6.29 (m, 1H), 5.09 (s, 1H), 3.96 (ddd, J=1.5, 7.3, 8.7, 1H), 3.50 (ddd, J=5.2, 8.7, 11.1, 1H), 2.91 (s, 3H), 2.29 (s, 6H), 2.14 (ddd, J=1.4, 5.2, 11.9, 1H), 2.04 (ddd, J=7.2, 11.3, 11.3, 1H), 1.46 (s, 3H); ¹³C NMR (125 MHz, CDCl₃): δ 152.5, 148.2, 142.4, 138.9, 137.4, 135.4, 125.5, 120.9, 116.6, 116.4, 105.4, 104.7, 67.4, 52.3, 41.6, 31.2, 24.6, 21.4; IR (film): 3308, 2923, 1748, 1497, 1195 cm⁻¹; HRMS-APCI (m/z) [M+H]⁺ calcd for C₂₁H₂₅N₂O₃⁺, 353.18597. found, 353.18496; [α]^{29.4} _{D} −39.34° (c=0.10, CH₂Cl₂).

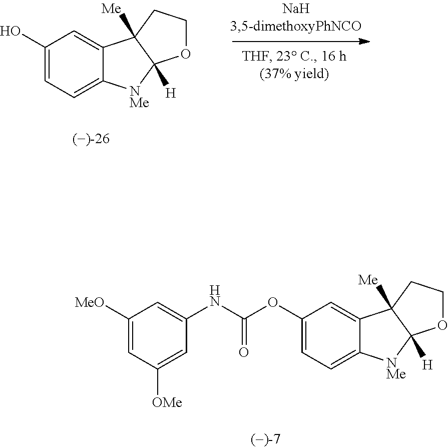 [SMILES: CN1c2ccc(O)cc2[C@]2(C)CCO[C@H]12 ; COc1cc(NC(=O)Oc2ccc3c(c2)[C@]2(C)CCO[C@@H]2N3C)cc(OC)c1]

Carbamate (−)-7. Following representative procedure A yielded carbamate (−)-7 (14.0 mg, 37% yield) as a white solid. Carbamate (−)-7: mp: 67-69° C.;

R_{f} 0.56 (1:1 Hexanes:EtOAc); ¹H NMR (500 MHz, CDCl₃): δ 6.30-6.83 (m, 3H), 6.72-6.65 (m, 2H), 6.30 (d, J=4.2, 1H), 6.21 (t, J=2.2, 1H), 5.08 (s, 1H), 3.96 (ddd, J=1.5, 7.4, 8.8, 1H), 3.77 (s, 6H), 3.50 (ddd, J=5.2, 8.7, 11.1, 1H), 2.91 (s, 3H), 2.13 (ddd, J=1.4, 5.2, 11.9, 1H), 2.04 (ddd, J=7.2, 11.3, 11.3, 1H), 1.45 (s, 3H); ¹³C NMR (125 MHz, CDCl₃): δ 161.2, 152.4, 148.3, 142.2, 139.4, 135.4, 120.9, 116.5, 105.4, 104.7, 96.8, 96.2, 67.3, 55.4, 52.3, 41.6, 31.2, 24.6; IR (film): 3306, 2936, 1749, 1615, 1202 cm⁻¹; HRMS-APCI (m/z) [M+H]⁺ calcd for C₂₁H₂₅N₂O₅⁺, 385.17580. found, 385.17580; [α]^{30.8}_{D} −56.00° (c=0.10, CH₂Cl₂).

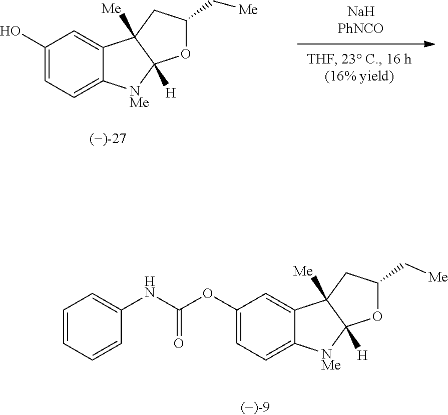 [SMILES: CC[C@H]1C[C@@]2(C)c3cc(O)ccc3N(C)[C@H]2O1 ; CC[C@H]1C[C@@]2(C)c3cc(OC(=O)Nc4ccccc4)ccc3N(C)[C@H]2O1]

Carbamate (−)-9. Following representative procedure A yielded carbamate (−)-9 (8.5 mg, 16% yield) as a clear oil. Carbamate (−)-9: R_{f} 0.73 (1:1 Hexanes:EtOAc); ¹H NMR (500 MHz, CDCl₃ δ 7.484-7.40 (m, 2H), 7.36-7.30 (m, 2H), 7.13-7.07 (m, 1H), 6.9-6.84 (m, 3H), 6.34 (d, J=8.4, 1H), 5.0 (s, 1H), 4.00 (ddd, J=6.8, 6.8, 13.6, 1H), 2.93 (s, 3H), 2.16 (dd, J=12.2, 6.6, 1H), 1.87 (dd, J=12.5, 6.9), 1.47-1.40 (m, 1H), 1.38 (s, 3H), 1.32-1.22 (m, 1H), 0.83 (t, J=7.6, 3H); ¹³C NMR (125 MHz, CDCl₃): 152.5, 146.8, 142.4, 137.6, 137.2, 129.1, 123.7, 120.6, 118.7, 116.4, 106.7, 105.7, 80.2, 52.1, 45.6, 31.8, 28.6, 24.2, 10.6; IR (film): 3313, 2961, 1722, 1496, 1198 cm⁻¹; HRMS-APCI (m/z) [M+H]⁺ calcd for C₂₁H₂₄N₂O₃, 353.18597. found 353.18462. [α]^{24.2} _{D} −49.33° (c=0.10, CH₂Cl₂).

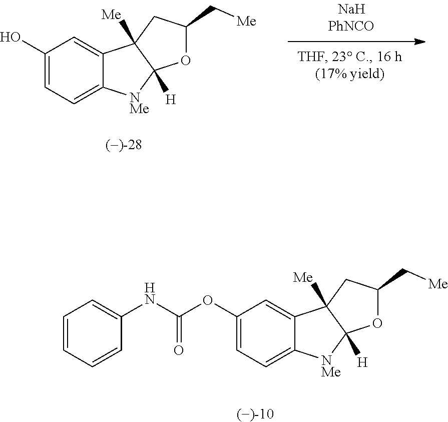 [SMILES: CC[C@@H]1C[C@@]2(C)c3cc(O)ccc3N(C)[C@H]2O1 ; CC[C@@H]1C[C@@]2(C)c3cc(OC(=O)Nc4ccccc4)ccc3N(C)[C@H]2O1]

Carbamate (−)-10. Following representative procedure A yielded carbamate (−)-10 (10.5 mg, 17% yield) as a clear oil. Carbamate (−)-10: R_{f}0.55 (5:1 Hexanes:EtOAc); ¹H NMR (500 MHz, CDCl₃): δ 7.47-7.40 (m, 2H), 7.37 (m, 2H), 7.13-7.06 (m, 1H), 6.93-6.83 (m, 3H), 6.30 (d, 1H), 5.10 (s, 1H), 3.68-3.60 (m, 1H), 2.91 (s, 3H), 2.20 (dd, J=4.4, 12.0, 1H), 1.72-1.54 (m, 3H), 1.53-1.46 (m, 1H), 1.44 (s, 3H), 0.87 (t, 3H); ¹³C NMR (125 MHz, CDCl₃): 152.5, 148.4, 142.1, 137.6, 135.9, 129.1, 123.8, 120.7, 118.7, 116.4, 105.0, 104.5, 80.1, 52.7, 47.1, 31.1, 27.5, 25.1, 10.3; IR (film): 3314, 2962, 1724, 1498, 1200 cm⁻¹; HRMS-APCI (m/z) [M+H]⁺ calcd for C₂₁H₂₄N₂O₃, 353.18597. found 353.18482. [α]^{21.4} _{D} −27.33° (c=0.10, CH₂Cl₂).

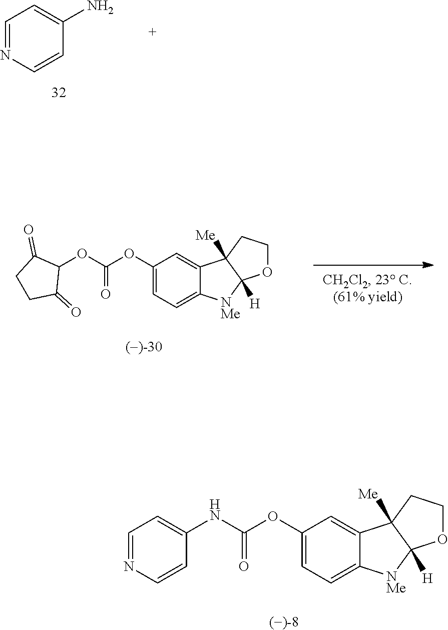 [SMILES: Nc1ccncc1 ; CN1c2ccc(OC(=O)OC3C(=O)CCC3=O)cc2[C@]2(C)CCO[C@H]12 ; CN1c2ccc(OC(=O)Nc3ccncc3)cc2[C@]2(C)CCO[C@H]12]

Carbamate (−)-8. Following representative procedure B yielded carbamate (−)-(8) (108.3 mg, 61% yield) as a white solid. Carbamate (−)-(8): mp: 164.0-166.0° C.; R_{f} 0.18 (1:4 Hexanes:EtOAc); ¹H NMR (500 MHz, CDCl₃): δ 8.50 (dd, J=1.6, 4.7, 2H), 7.39 (dd, J=1.6, 4.7, 2H), 7.22-7.20 (m, 1H), 6.89-6.86 (m, 2H), 6.31 (d, J=8.1, 1H), 5.10 (s, 1H), 3.97 (ddd, J=1.5, 7.1, 8.7, 1H), 3.50 (ddd, J=5.1, 8.7, 11.1, 1H), 2.91 (s, 3H), 2.13 (ddd, J=1.5, 5.1, 12.1, 1H), 2.04 (ddd, J=7.1, 11.1, 12.1, 1H), 1.46 (s, 3H); ¹³C NMR (125 MHz, CDCl₃): 152.1, 150.9, 148.7, 145.0, 141.9, 135.7, 120.8, 116.4, 112.7, 105.5, 104.7, 67.4, 52.5, 41.8, 31.2, 24.8; IR (film): 3165, 2960, 1749, 1594, 1494 cm⁻¹; HRMS-APCI (m/z) [M+H]⁺ calcd for C₁₈H₂₀N₃O₃, 326.14992. found 326.14971. Chiral Preparative SFC: 10×250 mm Chiral Technologies OD-H SFC column, 15% i-PrOH with 0.2% Et₃N (v/v), 30.0 mL/min, λ=254 nm, 40° C., nozzle pressure=100 bar CO₂, t_{R1}=4.9 min, [α]^{24.8} _{D} −50.00° (c=0.10, CH₂Cl₂); t_{R2}=5.4 min. [α]^{28.3} _{D}+62.000 (c=0.10, CH₂Cl₂).

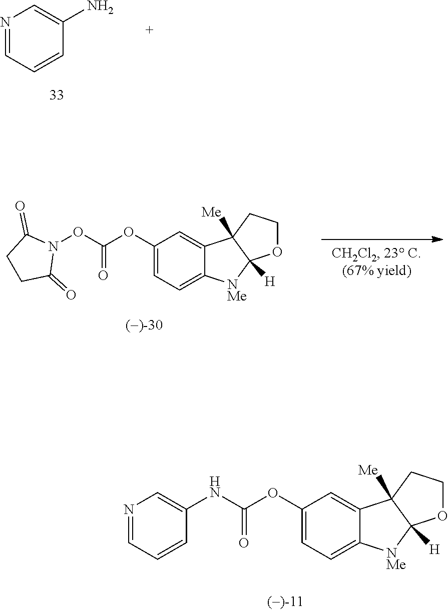 [SMILES: Nc1cccnc1 ; CN1c2ccc(OC(=O)ON3C(=O)CCC3=O)cc2[C@]2(C)CCO[C@H]12 ; CN1c2ccc(OC(=O)Nc3cccnc3)cc2[C@]2(C)CCO[C@H]12]

Carbamate (−)-11. Following representative procedure B yielded carbamate (−)-11 (7.5 mg, 67% yield) as a white solid. Carbamate (−)-11: mp: 131.0-133.6° C.;

R_{f} 0.24 (1:5 Hexanes:EtOAc); ¹H NMR (500 MHz, CDCl₃): δ 8.56 (d, J=8.6, 1H), 8.35 (dd, J=1.3, 4.8, 1H), 8.06 (br. d, J=6.6, 1H), 7.28 (dd, J=4.8, 8.6, 1H), 6.90-6.68 (m, 2H), 6.31 (d, J=8.1, 1H), 5.09 (s, 1H), 3.96 (ddd, J=1.3, 7.2, 8.6, 1H), 3.50 (ddd, J=5.1, 8.6, 11.0, 1H), 2.91 (s, 3H), 2.13 (ddd, J=1.3, 5.1, 12.0, 1H), 2.04 (ddd, J=7.2, 11.0, 12.0, 1H), 1.46 (s, 3H); ¹³C NMR (125 MHz, CDCl₃): δ 152.8, 148.6, 145.0, 142.2, 140.4, 135.7, 134.8, 126.0, 123.9, 120.9, 116.5, 105.5, 104.8, 67.5, 52.5, 41.8, 31.3, 24.8; IR (film): 3187, 2931, 1745, 1497, 1197 cm⁻¹; HRMS-APCI (m/z) [M+H]⁺ calcd for C₁₈H₂₀N₃O₃⁺, 326.14992. found, 326.15073; [α]_{D}^{24.3} −52.70° (c=0.10, CH₂Cl₂).

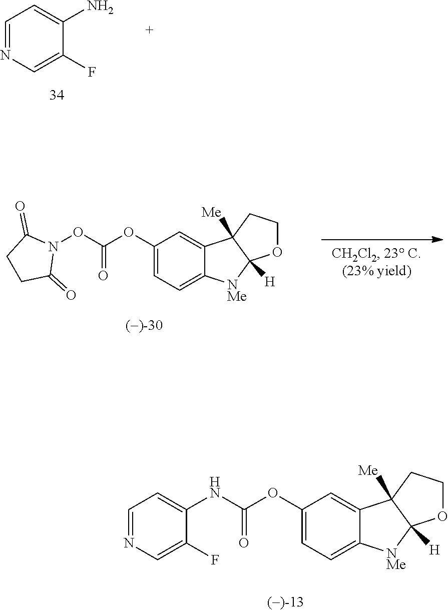 [SMILES: Nc1ccncc1F ; CN1c2ccc(OC(=O)ON3C(=O)CCC3=O)cc2[C@]2(C)CCO[C@H]12 ; CN1c2ccc(OC(=O)Nc3ccncc3F)cc2[C@]2(C)CCO[C@H]12]

Carbamate (−)-13. Following representative procedure B yielded carbamate (−)-13 (2.7 mg, 23% yield) as an amorphous solid. Carbamate (−)-13:

R_{f} 0.38 (1:5 Hexanes:EtOAc); ¹H NMR (500 MHz, CDCl₃): δ 8.43 (d, J=2.4, 1H), 8.34 (d, J=5.7, 1H), 8.14 (br. t, J=6.3, 1H), 7.34 (br. s, 1H), 6.91-6.87 (m, 2H), 6.32 (d, J=8.2, 1H), 5.10 (s, 1H), 3.97 (ddd, J=1.7, 7.3, 8.8, 1H), 3.50 (ddd, J=5.3, 8.8, 11.0, 1H), 2.92 (s, 3H), 2.13 (ddd, J=1.3, 5.3, 12.0, 1H), 2.05 (ddd, J=7.3, 11.0, 12.0, 1H), 1.47 (s, 3H); ¹³C NMR (125 MHz, CDCl₃): δ 151.7, 150.2, 148.8, 148.2, 147.1, 147.1, 141.8, 137.3, 137.1, 135.8, 133.6, 133.5, 120.8, 116.3, 113.4, 105.5, 104.7, 67.4, 52.5, 41.8, 31.2, 24.8; ¹⁹F-NMR (376 Hz, CDCl₃): δ−148.00; IR (film): 3238, 2926, 2870, 1753, 1620 cm⁻¹; HRMS-APCI (m/z) [M+H]⁺ calcd for C₁₈H₁₉N₃O₃F⁺, 344.14050. found, 344.14163; [α]^{28.4} _{D} −62.70° (c=0.10, CH₂Cl₂).

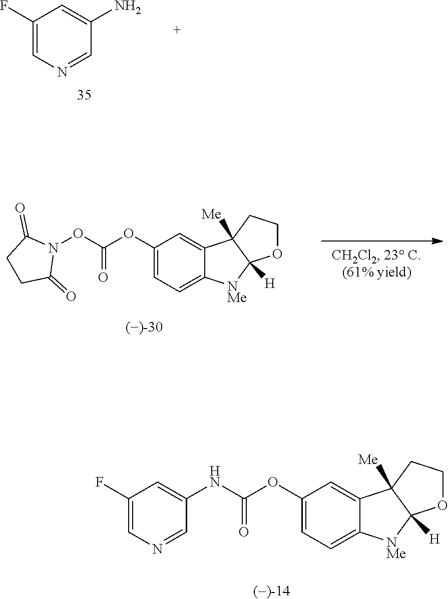 [SMILES: Nc1cncc(F)c1 ; CN1c2ccc(OC(=O)ON3C(=O)CCC3=O)cc2[C@]2(C)CCO[C@H]12 ; CN1c2ccc(OC(=O)Nc3cncc(F)c3)cc2[C@]2(C)CCO[C@H]12]

Carbamate (−)-14. Following representative procedure B yielded carbamate (−)-14 (6.0 mg, 61% yield) as a colorless oil. Carbamate (−)-14: R_{f} 0.38 (1:1 Hexanes:EtOAc); ¹H NMR (500 MHz, CDCl₃): δ 8.29-8.29 (m, 1H), 8.21 (d, J=2.5, 1H), 7.99 (br. d, J=9.3, 1H), 6.89-6.86 (m, 2H), 6.31 (d, J=8.3, 1H), 5.10 (s, 1H), 3.97 (ddd, J=1.6, 7.3, 8.7, 1H), 3.50 (ddd, J=5.2, 8.7, 11.1, 1H), 2.91 (s, 3H), 2.13 (ddd, J=1.3, 5.2, 12.1, 1H), 2.05 (ddd J=7.2, 11.1, 12.1, 1H), 1.47 (s, 3H); ¹³C NMR (125 MHz, CDCl₃): δ 160.8, 158.7, 152.5, 148.7, 142.0, 135.9, 135.9, 135.7, 133.1, 132.9, 128.5, 120.8, 116.4, 113.4, 113.2, 105.5, 104.7, 67.4, 52.5, 41.8, 31.2, 24.7; ¹⁹F-NMR (376 Hz, CDCl₃): δ −125.26 (d, J=10.5); IR (film): 3243, 2959, 2925, 1748, 1601, 1197 cm⁻¹; HRMS-APCI (m/z) [M+H]⁺ calcd for C₁₈H₁₉N₃O₃F₁⁺, 344.14050. found, 344.14028; [au]^{31.0} _{D} −57.33° (c=0.10, CH₂Cl₂).

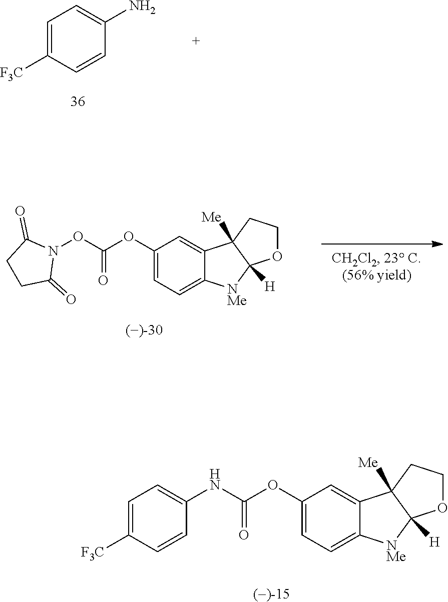 [SMILES: Nc1ccc(C(F)(F)F)cc1 ; CN1c2ccc(OC(=O)ON3C(=O)CCC3=O)cc2[C@]2(C)CCO[C@H]12 ; CN1c2ccc(OC(=O)Nc3ccc(C(F)(F)F)cc3)cc2[C@]2(C)CCO[C@H]12]

Carbamate (−)-15. Following representative procedure B yielded carbamate (−)-15 (6.3 mg, 56% yield) as a white solid. Carbamate (−)-15: mp: 193.0-195.0° C.;

R_{f} 0.72 (1:1 Hexanes:EtOAc); ¹H NMR (500 MHz, CDCl₃): δ 7.59-7.54 (m, 4H), 7.07 (br. s, 1H), 6.90-6.87 (m, 2H), 6.31 (d, J=8.4, 1H), 5.10 (s, 1H), 3.96 (ddd, J=1.5, 7.3, 8.8, 1H), 3.50 (ddd, J=5.2, 8.8, 11.1, 1H), 2.91 (s, 3H), 2.13 (ddd, J=1.5, 5.2, 12.0, 1H), 2.04 (ddd, J=7.3, 11.1, 12.0, 1H), 1.46 (s, 3H); ¹³C NMR (125 MHz, CDCl₃): δ 152.4, 148.6, 142.1, 140.9, 135.7, 127.5, 126.6 (q, J=3.7), 125.8, 125.6, 125.3, 123.2, 121.0, 120.9, 118.3, 116.5, 105.5, 104.8, 67.5, 52.5, 41.8, 31.3, 24.7; ¹⁹F-NMR (376 Hz, CDCl₃): δ −62.03; IR (film): 3352, 2885, 1719, 1615, 1117 cm⁻¹; HRMS-APCI (m/z) [M+H]⁺ calcd for C₂₀H₂₀N₂O₃F₃⁺, 393.14205. found, 393.14164; [α]^{30.7} _{D} −58.03° (c=0.10, CH₂Cl₂).

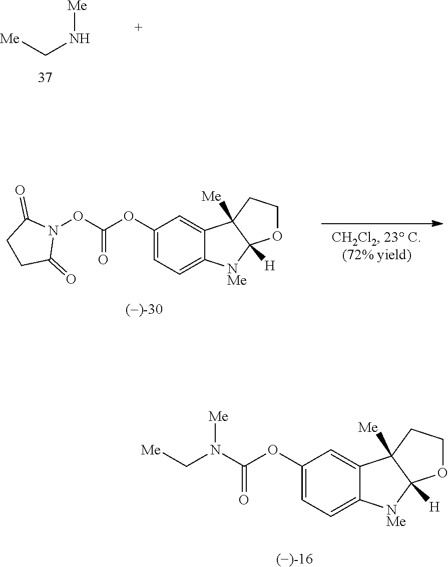

Carbamate (−)-16. Following representative procedure B yielded carbamate (−)-16 (6.0 mg, 72% yield) as a colorless oil. Carbamate (−)-16: R_{f} 0.54 (1:1 Hexanes:EtOAc); ¹H NMR (500 MHz, CDCl₃): δ 6.81 (app. d, J=8.3, 2H), 6.28 (d, J=8.7, 1H), 5.06 (s, 1H), 3.94 (ddd, J=1.5. 7.3, 8.7, 1H), 3.50-3.39 (m, 3H), 3.04-2.97 (m, 3H), 2.89 (s, 3H), 2.13 (ddd, J=1.5, 5.1, 12.0, 1H), 2.01 (ddd, J=7.3, 11.3, 12.0, 1H), 1.44 (s, 3H), 1.25-1.16 (m, 3H); ¹³C NMR (125 MHz, CDCl₃): δ 155.6, 155.4, 148.0, 143.4, 135.3, 121.0, 116.8, 116.8, 105.6, 104.9, 67.5, 52.5, 44.1, 41.7, 34.3, 33.8, 31.4, 24.7, 13.4, 12.7; IR (film): 2963, 2931, 1717, 1614, 1396 cm⁻¹; HRMS-APCI (m/z) [M+H]⁺ calcd for C₁₆H₂₃N₂O₃⁺, 291.17032. found, 291.16895; [α]^{30.9} _{D} −53.97° (c=0.10, CH₂Cl₂).

### Example 3: Preparation of Exemplary Compounds Via Flow Chemistry

The approach to obtain indoline scaffolds in the microfluidic reactor using the interrupted Fischer indole synthesis is shown below.

As part of developing the transformation was initially tried with the reaction of phenyl-hydrazine (4) and lactol 5 in the reactor using (1:1) AcOH—H₂O at 60° C. and 1 bar pressure to determine if it was possible to obtain furoindoline 6. Unfortunately, no product was observed. Next the temperature, pressure and retention time in the microfluidic reactor was modulated. After several variations it was discovered that successful interrupted Fischer indolization reaction occurred at 80° C., 2 bar pressure, and 20 min retention time in the microfluidic reactor affording the desired furoindoline 6 in 84% yield (entry 5 of Table 2). Surprisingly, further increasing the temperature, pressure and retention time in the microfluidic reactor led to the optimal conditions of 120° C., 3 bar pressure and 5 min retention time in the microfluidic reactor affording a 97% yield of 6 as determined by HPLC (entry 8). Scale up and purification of the product by flash chromatography gave 85% isolated yield of furoindoline 6 in 85% isolated yield.

TABLE 2
Optimization of interrupted Fischer indolization in a microfluidic reactor^{a}

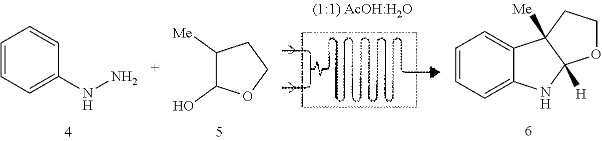 [SMILES: NNc1ccccc1 ; CC1CCOC1O ; C[C@@]12CCO[C@@H]1Nc1ccccc12]

| | | | | Reten- | |
|---|---|---|---|---|---|
| | | Temper- | Flow | tion | |
| | Pressure | ature | Rate^{b} | Time^{c} | Yield^{d} |
| Entry | (bar) | (° C.) | (μL/min) | (min) | (%) |
| 1^{e} | 1 | 60 | 500 | 2 | 0 |
| 2^{e} | 2 | 70 | 500 | 2 | 0 |
| 3^{e} | 2 | 70 | 200 | 5 | 0 |
| 4^{e} | 2 | 80 | 200 | 5 | 0 |
| 5^{e} | 2 | 80 | 50 | 20 | 84 |
| 6^{e} | 2 | 100 | 50 | 20 | 97 |
| 7^{f} | 3 | 120 | 100 | 10 | 97 |
| 8^{f} | 3 | 120 | 200 | 5 | 97^{g} |

^{a} Synthesis of compound 6 was performed under varying conditions (entry 1-8); two solutions were prepared and introduced by Pump A & B into the Asia microfluidic reactor; one contained the phenyl hydrazine 4 (1.0 equivalent) in AcOH—H₂O (2.5 mL, 1:1 v/v) and the other contained latent aldehyde 5 (1.1 equivalent) in AcOH—H₂O (2.5 mL, 1:1 v/v). ^{b} Combined flow rate from both pumps. ^{c} Residence time in microfluidic chip reactor (1000 μL volume). ^{d} HPLC yield from microfluidic flow reactor. ^{e} Hydrazine added at 0.2 M. Latent aldehyde at 1.1 eqiv. ^{f} Hydrazine added at 0.5 M. Latent aldehyde at 1.1 eqiv. ^{g} Scale-up of reaction yielded furoindoline 6 in 85% yield.

After identifying the optimal microfluidic conditions for the interrupted Fischer indolization, the reaction was performed using different hydrazines while keeping the latent aldehyde (5) constant. As shown in Table 3, the reaction is broad in scope with respect to the hydrazine surrogates. Both para- and ortho-substituents were tolerated under the microfluidic reaction condition (entries 1, 2, 4, 5 and 6) and afforded the corresponding furoindoline products in good yields. The N-methyl-substituted hydrazine 9 was shown to be a competent coupling partner (entry 3) and afforded the desired N-methyl furoindoline 16 in 68% yield.

TABLE 3
Hydrazine variants in the microfluidic interrupted Fischer indolization reaction.^{a}

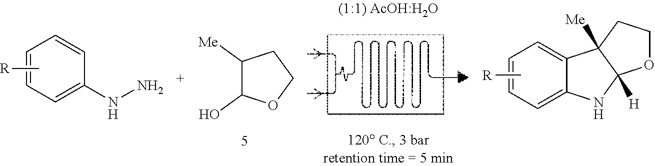

| | Hydrazine | Product | Yield |
|---|---|---|---|
| Entry | Variant | | |
| 1 | 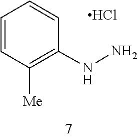 | 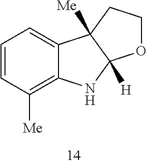 [SMILES: Cc1cccc2c1N[C@H]1OCC[C@@]21C] | 78% |
| 2 | 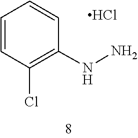 | 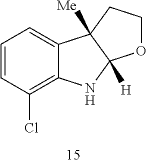 [SMILES: C[C@@]12CCO[C@@H]1Nc1c(Cl)cccc12] | 57% |
| 3 | 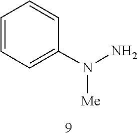 | 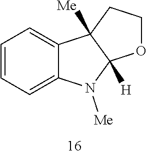 [SMILES: CN1c2ccccc2[C@]2(C)CCO[C@H]12] | 68% |
| 4 | 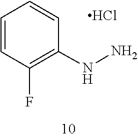 | 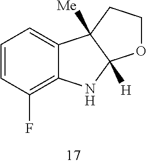 [SMILES: C[C@@]12CCO[C@@H]1Nc1c(F)cccc12] | 34% |
| 5 | 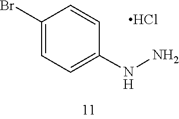 | 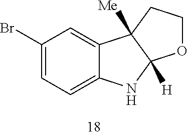 [SMILES: C[C@@]12CCO[C@@H]1Nc1ccc(Br)cc12] | 55% |
| 6 | 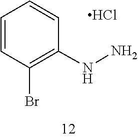 | 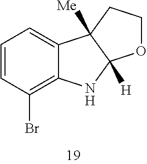 [SMILES: C[C@@]12CCO[C@@H]1Nc1c(Br)cccc12] | 56% |
| 7 | 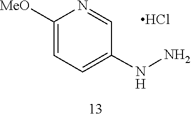 | 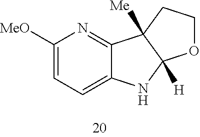 [SMILES: COc1ccc2c(n1)[C@]1(C)CCO[C@@H]1N2] | 40% |

^{a}The reactions were performed using optimized condition (see Table 2, entry 8); two solutions were prepared and introduced by Pump A & B into the Asia microfluidic reactor; one contained the different hydrazine surrogates (7-13) (1.0 equivalent) in AcOH—H₂O (2.5 mL, 1:1 v/v) and the other contained latent aldehyde 5 (1.1 equivalent) in AcOH—H₂O (2.5 mL, 1:1 v/v). Average isolated yields from at least two trials for products (14-20) are reported.
The p-methoxypyridylhydrazine salt 13 afforded the furanoazaindoline 20 in moderate 40% yield (entry 7). Several of the products such as the haloindolines (entries 2, 5, 6), are setup for further functionalization by transition-metal-catalyzed cross-coupling chemistry.

The scope of the transformation with nitrogen- or oxygen-containing latent aldehyde coupling partners was further evaluated by using phenyl hydrazine 4 (Table 5). The use of five-membered oxygen- or nitrogen-containing latent aldehydes afforded the corresponding furoindoline 24 and pyrrolidinoindoline 25 in 49% and 42% yield, respectively. Surprisingly, even the six-membered homolog (26) of the pyrrolidinoindoline framework was obtained using this methodology, although in lower yield.

TABLE 4
Variations of the latent aldehyde surrogate in the microfluidic reaction ^{a}

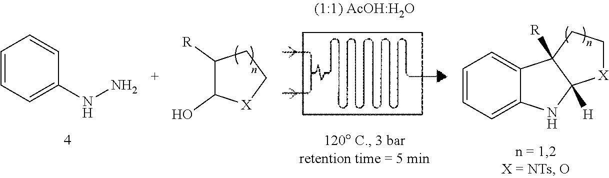

| Entry | Lactol Variant | Product | Yield |
|---|---|---|---|
| 1 | 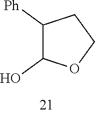 [SMILES: OC1OCCC1c1ccccc1] | 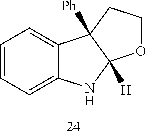 [SMILES: c1ccc([C@@]23CCO[C@@H]2Nc2ccccc23)cc1] | 49% |
| 2 | 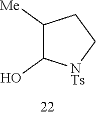 [SMILES: Cc1ccc(S(=O)(=O)N2CCC(C)C2O)cc1] | 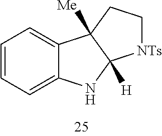 [SMILES: Cc1ccc(S(=O)(=O)N2CC[C@@]3(C)c4ccccc4N[C@@H]23)cc1] | 42% |
| 3 | 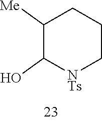 [SMILES: Cc1ccc(S(=O)(=O)N2CCCC(C)C2O)cc1] | 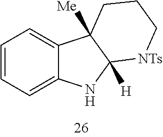 [SMILES: Cc1ccc(S(=O)(=O)N2CCC[C@@]3(C)c4ccccc4N[C@@H]23)cc1] | 21% |

^{a} The reactions were performed using optimized conditions (see Table 2, entry 8); two solutions were prepared and introduced by Pump A & B into the Asia microfluidic reactor; one contained the phenyl hydrazine (4) (1.0 equivalent) in AcOH—H₂O (2.5 mL, 1:1 v/v) and the other contained lactol variant (21-23) (1.1 equivalent) in AcOH—H₂O (2.5 mL, 1:1 v/v). Average isolated yields from at least two trials for products (24-26) are reported.
^{b} Reaction performed in AcOH.

In summary, the use of a microfluidic reactor for the interrupted Fischer indolization resulted in short reaction times and led to indoline and azaindoline products in good yields. Unexpectedly, it was possible to accelerate the microfluidic reaction by increasing temperature and pressure to achieve yields up to 85% yield with 5 minutes residence time in the reactor. This rapid green chemistry methodology should facilitate continuous synthesis of fused indoline ring systems that can be coupled with additional microfluidic reactors for multistep synthesis. This approach allows for rapid synthesis of these important scaffolds as dual enzyme inhibitors and will prove useful for the discovery of new drug candidates.

### A. Fischer Indolization

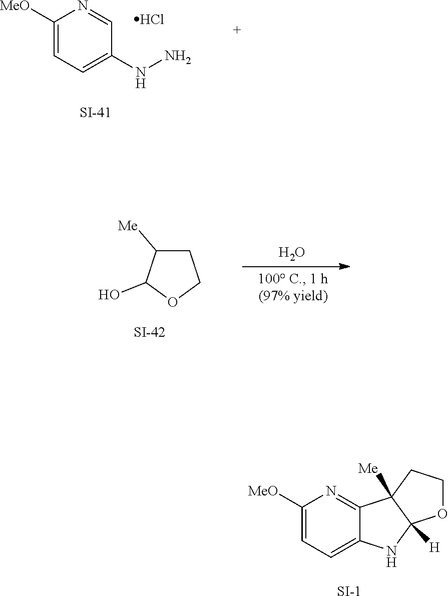

Representative Procedure (azaindoline SI-1 is used as an example). Azaindoline SI-1. A scintillation vial containing a magnetic stir bar was charged with lactol SI-42 (23.6 mg, 0.231 mmol, 1.0 equiv) and deionized H₂O (4.6 mL, 0.05 M). Hydrazine SI-41 (60.9 mg, 0.347 mmol, 1.5 equiv) was added and the vial was capped with a Teflon-lined screw cap. The reaction mixture was then placed in a pre-heated aluminum block and allowed to stir at 100° C. for 1 h. After cooling to room temperature, the reaction mixture was transferred to a separatory funnel with deionized H₂O (3 mL) and EtOAc (3 mL). The reaction mixture was then basified with 40% w/w KOH in deionized H₂O (4 mL) to a pH of 12. The layers were separated and the aqueous layer was extracted with EtOAc (3×5 mL). The combined organic layers were washed with saturated aqueous NaCl (5 mL), and dried over Na₂SO₄. The volatiles were removed under reduced pressure, and the crude residue was purified by preparative thin-layer chromatography (2:1 Hexanes:EtOAc) to yield azaindoline SI-1 (97% yield) as a yellow oil. Azaindoline SI-1: ¹H NMR (500 MHz, CDCl₃): δ 6.88 (d, J=8.4, 1H), 6.43 (d, J=8.4, 1H), 5.27 (s, 1H), 3.96 (ddd, J=8.9, 7.6, 1.7, 1H), 3.87 (s, 3H), 3.53 (ddd, J=10.9, 8.9, 5.3, 1H), 2.40 (ddd, J=12.1, 5.3, 1.7, 1H), 2.03 (ddd, J=12.1, 10.9, 7.6, 1H) 1.48 (s, 3H).

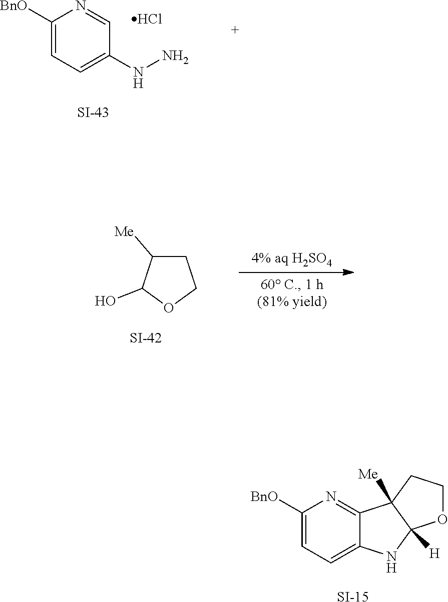

Azaindoline SI-15. Purification by flash chromatography (2:1 Hexanes:EtOAc) yielded azaindoline SI-15 (81% yield) as a yellow oil. Azaindoline SI-15: ¹H NMR (500 MHz, CDCl₃): δ 7.46 (d, J=7.2, 2H), 7.35 (t, J=7.2, 2H), 7.30-7.28 (m, 1H), 6.87 (d, J=8.5, 1H), 6.48 (d, J=8.5, 1H), 5.31 (s, 1H), 5.30 (s, 1H), 5.27 (s, 1H), 4.25 (br. s, 1H), 3.97 (ddd, J=9.1, 7.7, 1.7, 1H), 3.53 (ddd, J=11.1, 9.1, 5.1. 1H), 2.39 (ddd, J=12.1, 5.1, 1.7, 1H), 2.04 (ddd, J=12.1, 11.1 7.7, 1H), 1.48 (s, 3H).

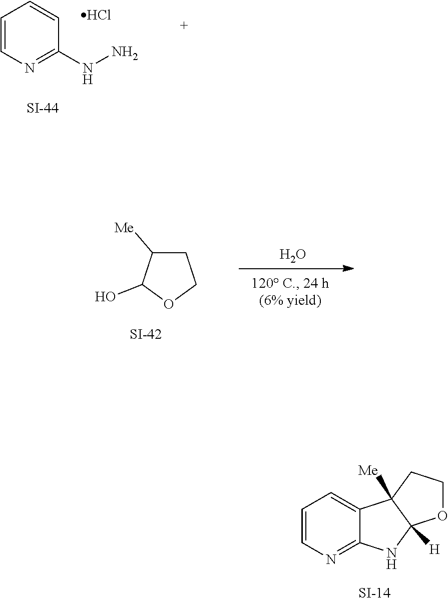

Azaindoline SI-14. Purification by flash chromatography (EtOAc, 2% Et₃N) yielded azaindoline SI-14 (6% yield) as a white solid. Azaindoline SI-14: ¹H NMR (500 MHz, CDCl₃): δ 7.88 (dd, J=5.3, 1.5, 1H), 7.26 (dd, J=7.1, 1.5), 6.59 (dd, J=5.3, 7.1, 1H), 5.35 (br. s, 1H), 5.31 (s, 1H), 3.98 (ddd, J=8.6, 7.1, 1.6, 1H), 3.57 (ddd, J=11.0, 8.6, 5.2, 1H), 2.14-2.04 (m, 2H), 1.48 (s, 3H).

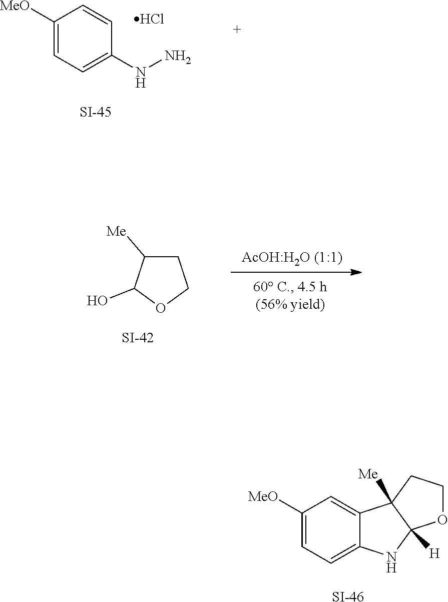

Indoline SI-46. Purification by flash chromatography (3:1 Hexanes:EtOAc→1:1 Hexanes:EtOAC) yielded indoline SI-46 (56% yield) as a yellow oil. Indoline SI-46: ¹H NMR (500 MHz, CDCl₃): δ 6.69 (d, J=2.3, 1H), 6.63 (dd, J=8.5, 2.8, 1H), 6.53 (d, J=8.3), 5.26 (s, 1H), 4.36 (br. S, 1H), 3.95 (ddd, J=8.6, 7.3, 1.4, 1H), 3.76 (s, 3H), 3.56 (ddd, J=5.1, 8.6, 10.9, 1H), 2.17 (ddd, J=1.4, 5.1, 11.9, 1H), 2.10-2.04 (m, 1H), 1.47 (s, 3H).

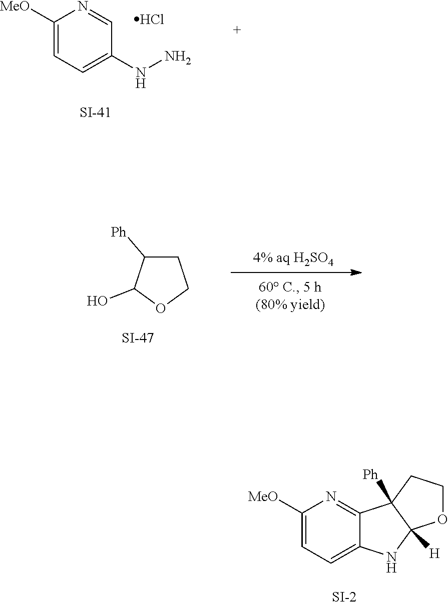

Azaindoline SI-2. Purification by preparative thin-layer chromatography (2:1 Hexanes:EtOAc) yielded azaindoline SI-2 (80% yield) as an amorphous solid. Azaindoline SI-2: ¹H NMR (500 MHz, CDCl₃): δ 7.42 (d, J=7.8, 2H), 7.32 (t, J=7.8, 2H), 7.26-7.21 (m, 1H), 6.94 (d, J=8.6, 1H), 6.47 (d, J=8.6, 1H), 5.69 (s, 1H), 4.39 (s, 1H), 4.18-4.15 (m, 1H), 3.85 (s, 3H), 3.67-3.62 (m, 1H), 2.85-2.82 (m, 1H), 2.65-2.59 (m, 1H).

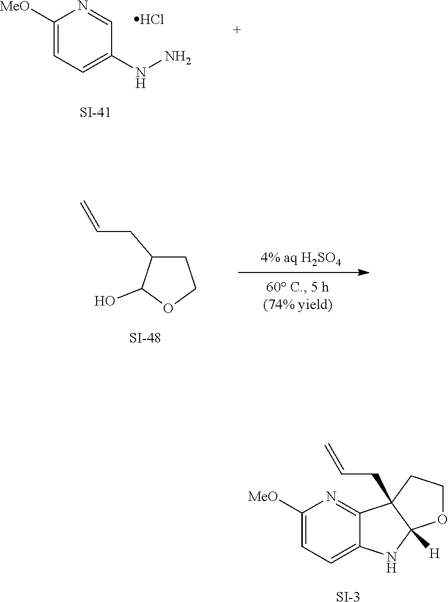

Azaindoline SI-3. Purification by preparative thin-layer chromatography (3:2 Hexanes:EtOAc) yielded azaindoline SI-3 (74% yield) as a red oil. Azaindoline SI-3: ¹H NMR (400 MHz, CDCl₃): δ 6.88-6.86 (m, 1H), 6.45-6.42 (m, 1H), 5.75-5.65 (m, 1H), 5.39 (s, 1H), 5.10-5.06 (m, 1H), 5.03-4.99 (m, 1H), 3.98-3.93 (m, 1H), 3.87 (s, 3H), 3.53 (ddd, J=11.2, 8.8, 5.3, 1H), 2.75-2.69 (m, 1H), 2.52-2.47 (m, 1H), 2.34-2.29 (m, 1H), 2.09 (app. ddd, J=11.2, 11.2, 7.4, 1H).

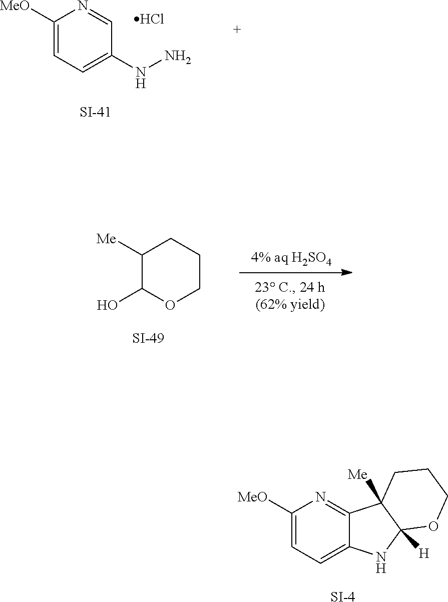

Azaindoline SI-4. Purification by preparative thin-layer chromatography (3:2 Hexanes:EtOAc) yielded azaindoline SI-4 (62% yield) as an amorphous solid. Azaindoline SI-4: ¹H NMR (400 MHz, CDCl₃): δ 6.93 (d, J=8.1, 1H), 6.41 (d, J=8.1, 1H), 4.74 (s, 1H), 4.04 (br. s, 1H), 3.87 (s, 3H), 3.75 (dddd, J=11.2, 4.0, 4.0, 1.6, 1H), 3.40 (ddd, J=11.2, 10.0, 3.0, 1H), 2.45 (dddd, J=13.3, 4.0, 4.0, 1.6, 1H), 1.62 (ddd, J=13.6, 11.8, 4.7, 1H), 1.55-1.47 (m, 1H), 1.47-1.36 (m, 1H), 1.17 (s, 3H).

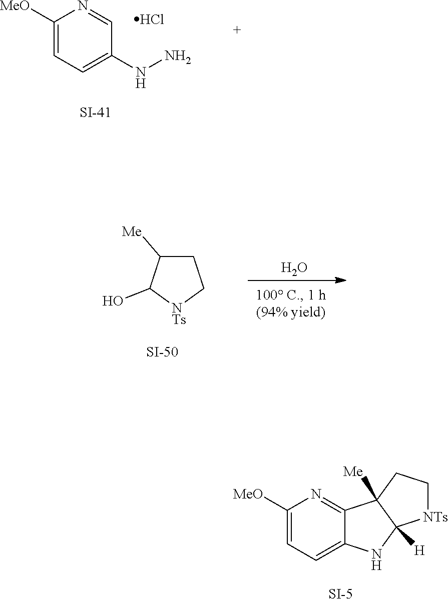

Azaindoline SI-5. Purification by preparative thin-layer chromatography (2:1 Hexanes:EtOAc) yielded azaindoline SI-5 (94% yield) as a colorless oil. Azaindoline SI-5: ¹H NMR (400 MHz, CDCl₃): δ 7.74 (d, J=8.3, 2H), 7.32 (d, J=8.3, 2H), 6.90 (d, J=8.4, 1H), 6.44 (d, J=8.4, 1H), 4.98 (s, 1H), 3.84 (s, 3H), 3.39 (ddd, J=10.5, 8.2, 2.3, 1H), 3.10 (ddd, J=10.5, 10.5, 6.4, 1H), 2.44 (s, 3H), 2.41 (ddd, J=12.5, 6.4, 2.3, 1H), 1.70 (ddd, J=12.5, 10.5, 8.2, 1H), 1.27 (s, 3H).

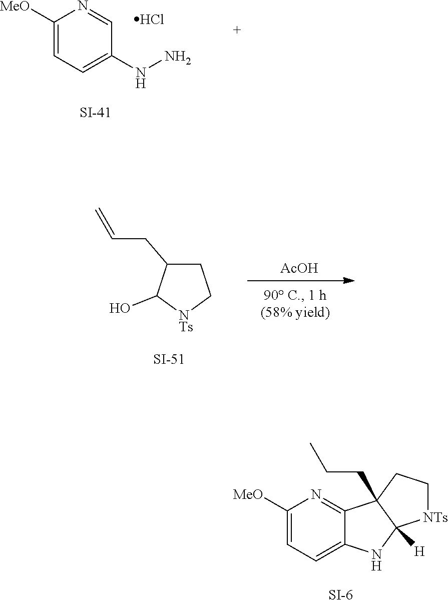

Azaindoline SI-6. Purification by preparative thin-layer chromatography (2:1 Hexanes:EtOAc) yielded azaindoline SI-6 (58% yield) as a colorless oil. Azaindoline SI-6: ¹H NMR (500 MHz, CDCl₃): δ 7.74 (d, J=7.8, 2H), 7.32 (d, J=7.8, 2H), 6.89 (d, J=8.4, 1H), 6.45 (d, J=8.4, 1H), 5.54 (dddd, J=17.4, 10.0, 7.4, 7.4, 1H), 5.11 (s, 1H), 4.98-4.93 (m, 2H), 4.52 (br. s, 1H), 3.84 (s, 3H), 3.38 (ddd, J=10.3, 8.3, 2.3, 1H), 3.10 (ddd, J=10.3, 10.3, 6.2, 1H), 2.44 (s, 3H), 2.41-2.38 (m, 1H), 2.32-2.27 (m, 2H), 1.78 (ddd, J=12.2, 10.2, 8.3, 1H).

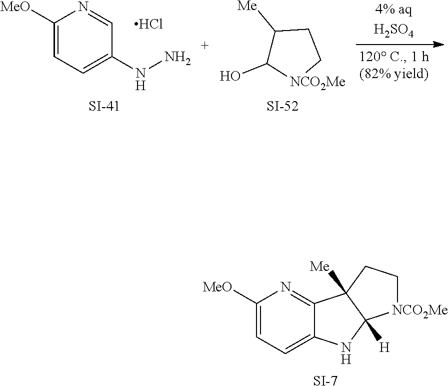

Azaindoline SI-7. Purification by preparative thin-layer chromatography (1:1 Hexanes:EtOAc) yielded azaindoline SI-7 (82% yield) as an amorphous solid. Azaindoline SI-7: ¹H NMR (500 MHz, CDCl₃, 58° C.): δ 6.85 (d, J=8.3, 1H), 6.43 (d, J=8.3, 1H), 5.10 (br. s, 1H), 3.88 (s, 3H), 3.72-3.64 (m, 4H), 3.09-3.04 (m, 1H), 2.53-2.49 (m, 1H), 2.02-1.95 (m, 1H), 1.42 (s, 3H).

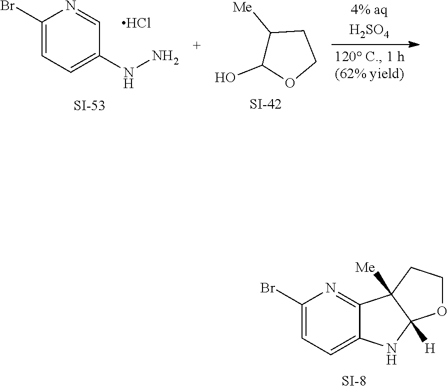

Azaindoline SI-8. Purification by preparative thin-layer chromatography (1:1 Hexanes:EtOAc) yielded azaindoline SI-8 (62% yield) as a light brown solid. Azaindoline SI-8: ¹H NMR (500 MHz, CDCl₃): δ 7.08 (d, J=8.3, 1H), 6.89 (d, J=8.3, 1H), 5.29 (s, 1H), 4.64 (br. s, 1H), 3.98 (ddd, J=8.9, 7.6, 1.6, 1H), 3.52 (dddd, J=11.3, 8.9, 5.3, 1H), 2.43 (ddd, J=12.3, 5.3, 1.6, 1H), 2.05 (ddd, J=12.3, 11.3, 7.6, 1H), 1.50 (s, 3H).

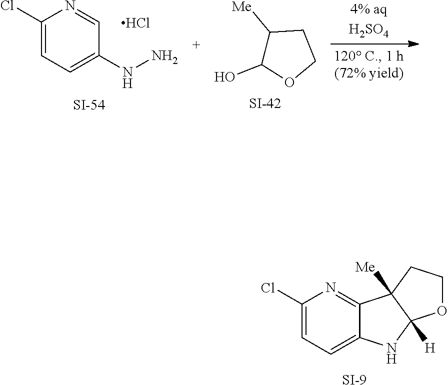

Azaindoline SI-9. Purification by preparative thin-layer chromatography (1:1 Hexanes:EtOAc) yielded azaindoline SI-9 (72% yield) as a brown solid. Azaindoline SI-9: ¹H NMR (500 MHz, CDCl₃): δ 6.95 (d, J=8.3, 1H), 6.77 (d, J=8.3, 1H), 5.31 (s, 1H), 4.62 (br. s, 1H), 3.99 (ddd, J=8.9, 7.6, 1.6, 1H), 3.53 (ddd, J=11.3, 8.9, 5.3, 1H), 2.43 (ddd, J=12.3, 5.3, 1.6, 1H), 2.06 (ddd, J=12.3, 11.3, 7.6, 1H), 1.51 (s, 3H).

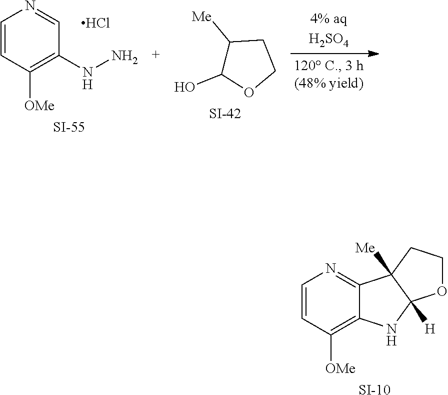

Azaindoline SI-10. Purification by preparative thin-layer chromatography (EtOAc) yielded azaindoline SI-10 (48% yield) as an amorphous solid. Azaindoline SI-10: ¹H NMR (500 MHz, CDCl₃): δ 7.94 (d, J=5.3, 1H), 6.61 (d, J=5.3, 1H), 5.33 (s, 1H), 4.51 (br. s, 1H), 3.99 (ddd, J=8.8, 7.6, 1.5, 1H), 3.88 (s, 3H), 3.54 (ddd, J=11.2, 8.8, 5.5, 1H), 2.45 (ddd, J=12.2, 5.5, 1.5, 1H), 2.08 (ddd, Js=12.2, 11.2, 7.6, 1H), 1.53 (s, 3H).

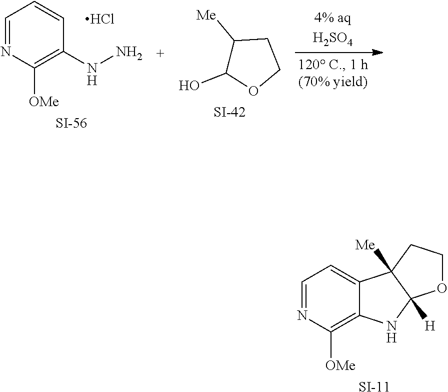

Azaindoline SI-11. Purification by preparative thin-layer chromatography (2:1 Hexanes:EtOAc) yielded azaindoline SI-11 (70% yield) as a colorless solid. Azaindoline SI-11: ¹H NMR (500 MHz, CDCl₃): δ 7.64 (d, J=5.1, 1H), 6.70 (d, J=5.1, 1H), 5.34 (s, 1H), 4.48 (br. s, 1H), 3.99-3.95 (m, 4H), 3.54 (ddd, J=11.2, 8.7, 5.1, 1H), 2.16 (ddd, J=12.3, 5.1, 1.6, 1H), 2.07 (ddd, J=12.3, 11.2, 7.3, 1H), 1.47 (s, 3H).

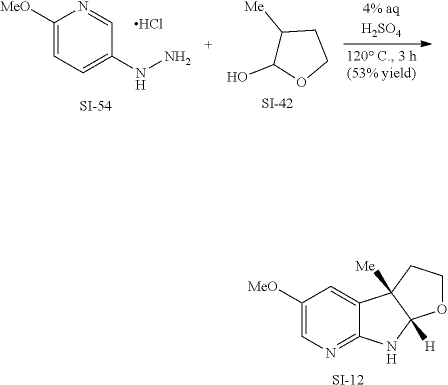

Azaindoline SI-12. Purification by preparative thin-layer chromatography (1:1 Hexanes:EtOAc) yielded azaindoline SI-12 (53% yield) as a yellow solid. Azaindoline SI-12: ¹H NMR (500 MHz, CDCl₃): δ 7.56 (d, J=2.7, 1H), 6.95 (d, J=2.7, 1H), 5.31 (s, 1H), 5.11 (br. s, 1H), 3.97 (ddd, J=8.8, 7.0, 1.6, 1H), 3.78 (s, 3H), 3.58 (ddd, J=10.9, 8.8, 5.4, 1H), 2.13-2.03 (m, 2H), 1.47 (s, 3H)

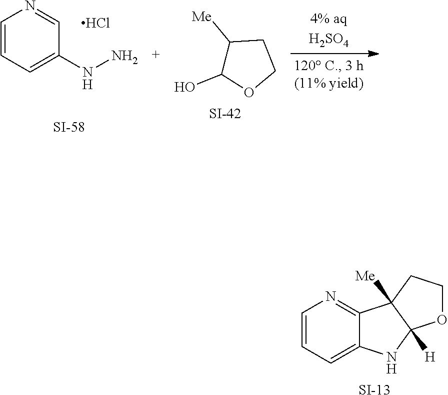

Azaindoline SI-13. Purification by preparative thin-layer chromatography (EtOAc, 2% Et₃N) yielded azaindoline SI-13 (11% yield) as an amorphous solid. Azaindoline SI-13: ¹H NMR (500 MHz, CDCl₃): δ 7.95 (dd, J=5.1, 1.4, 1H), 6.93 (dd, J=7.9, 5.1, 1H), 6.79 (dd, J=7.9, 1.4, 1H), 5.31 (s, 1H), 4.59 (br. s, 1H), 3.99 (ddd, J=8.9, 7.6, 1.6, 1H), 3.53 (ddd, J=11.2, 8.9, 5.3, 1H), 2.43 (ddd, J=12.2, 5.3, 1.6, 1H), 2.09 (ddd, J=12.2, 11.2, 7.6, 1H), 1.52 (s, 3H).

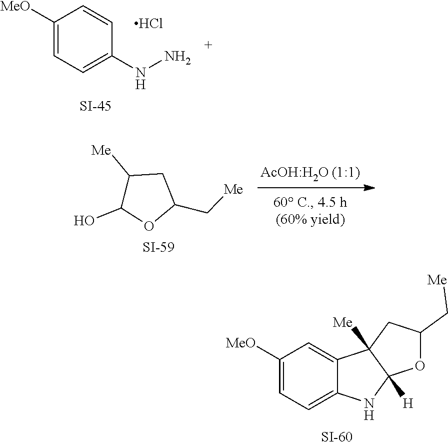

Indoline SI-60. Purification by preparative thin-layer chromatography (1:1 Hexanes:EtOAc) yielded indoline SI-60 (60% yield) in a 1.5:1 mixture of diastereomers as an amorphous solid. Indoline SI-60: ¹H NMR (500 MHz, CDCl₃): δ 6.69-6.67 (6.69-6.67) (m, 1H), 6.65-6.62 (6.65-6.62) (m, 1H), 6.56 (6.53) (d, J=8.5, 1H), 5.14 (5.29) (s, 1H), 3.95-3.89 (3.72-3.69) (m, 1H), 3.76 (3.76) (s, 3H), 2.17 (2.23) (dd, J=12.0, 6.1, 1H), 1.87 (dd, J=12.0, 7.5, 1H), 1.68-1.63 (1.68-1.63) (m, 1H), 1.52-1.46 (1.52-1.46) (m, 1H), 1.37 (1.45) (s, 3H), 1.33-1.24 (1.33-1.24) (m, 1H), 0.89-0.84 (0.89-0.84) (m, 3H).

### B. Indoline Alkylation

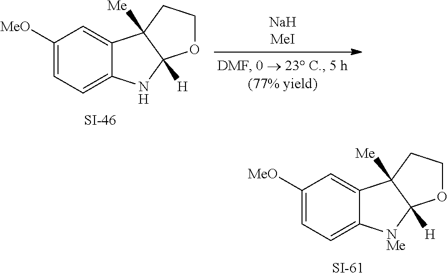

Representative Procedure (Indoline SI-61 is used as an example). Indoline SI-61. A 1-dram vial was charged with a magnetic stir bar, flame-dried under reduced pressure, and allowed to cool under a N₂ atmosphere. Indoline SI-46 (83.0 mg, 0.294 mmol, 1.0 equiv) was added and the vial was flushed with N₂ for 5 min. DMF (294 uL, 1.0 M) was added and the reaction mixture cooled to 0° C. under an N₂ atmosphere. NaH (60% dispersion in mineral oil, 26.0 mg, 0.647 mmol, 2.2 equiv) was added in one portion and the reaction was left to stir for 30 min at 0° C. MeI (44 uL, 0.706 mmol, 2.4 equiv) was then added dropwise over 1 min. After 30 min stirring at 0° C., the reaction mixture was warmed to 23° C. and allowed to stir for 4 h. The reaction mixture was then transferred to a separatory funnel with deionized H₂O (3 mL) and CH₂Cl₂ (3 mL). The layers were separated and the aqueous layer was extracted with CH₂Cl₂ (3×5 mL). The combined organic layers were washed with deionized H₂O (3×3 mL), saturated aqueous NaCl (10 mL), and dried over Na₂SO₄. The volatiles were removed under reduced pressure, and the crude residue was purified by flash chromatography (3:1 Hexanes:EtOAc) to yield indoline SI-61 (61.0 mg, 77% yield) as a colorless oil. Indoline SI-61: ¹H NMR (500 MHz, CDCl₃): δ 6.69 (d, J=2.3, 1H), 6.66 (dd, J=8.3, 2.7, 1H), 6.28 (d, J=8.3, 1H), 5.03 (s, 1H), 3.94 (ddd, J=8.9, 7.5, 1.6, 1H), 3.75 (s, 3H), 3.47 (ddd, J=10.8, 8.5, 5.3, 1H), 2.88 (s, 3H), 2.12 (ddd, J=12.1, 5.3, 1.6, 1H), 2.07-2.01 (m, 1H), 1.44 (s, 3H).

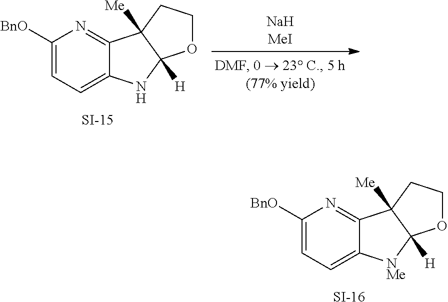

Azaindoline SI-16. Purification by flash chromatography (3:1 Hexanes:EtOAc) yielded azaindoline SI-16 (77% yield) as a colorless oil. Azaindoline SI-16: ¹H NMR (500 MHz, CDCl₃): δ 7.46 (d, J=7.5, 2H), 7.35 (t, J=7.5, 2H), 7.30-7.27 (m, 1H), 6.64 (d, J=8.3, 1H), 6.50 (d, J=8.3, 1H), 5.32-5.27 (m, 2H), 5.04 (s, 1H), 3.96 (ddd, J=9.0, 7.6, 1.6, 1H), 3.43 (ddd, J=11.2, 9.0, 5.6, 1H), 2.88 (s, 3H), 2.36 (ddd, J=12.2, 5.6, 1.6, 1H), 2.02 (ddd, J=12.2, 11.2, 7.6, 1H), 1.47 (s, 3H).

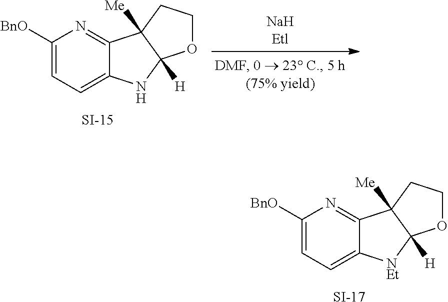

Azaindoline SI-17. Purification by flash chromatography (3:1 Hexanes:EtOAc) yielded azaindoline SI-17 (75% yield) as a colorless oil. Azaindoline SI-17: ¹H NMR (500 MHz, CDCl₃): δ 7.46 (d, J=7.5, 2H), 7.35 (t, J=7.5, 2H), 7.28 (t, J=7.5, 1H), 6.66 (d, J=8.5, 1H), 6.49 (t, J=8.5, 1H), 5.28 (d, J=3.0, 2H), 5.15 (s, 1H), 3.94 (ddd, J=8.0, 7.5, 1.4, 1H), 3.44 (ddd, J=11.2, 8.8, 5.4, 1H), 3.28 (dq, J=14.1, 7.1, 2H), 2.35 (ddd, J=12.1, 5.4, 1.4, 1H), 2.00 (app. ddd, J=11.2, 11.2, 7.5, 1H), 1.46 (s, 3H), 1.21 (t, J=7.1, 3H).

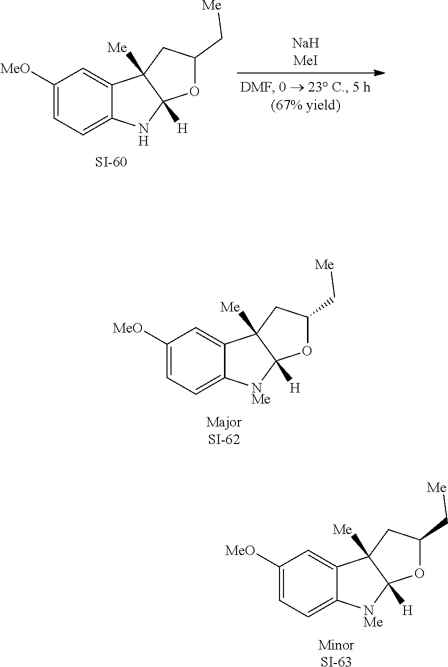

Indoline SI-62 and SI-63. Purification by preparative thin-layer chromatography (10:1 Hexanes:EtOAc) yielded indoline SI-62 and SI-63 (67% yield) in a 1.5:1 ratio of diastereomers as amorphous solids. Indoline SI-62: ¹H NMR (500 MHz, CDCl₃, major product): δ 6.68-6.66 (m, 2H), 6.31 (d, J=8.1, 1H), 4.94 (s, 1H), 3.76 (s, 3H), 2.89 (s, 3H), 2.15 (dd, J=12.3, 6.8, 1H), 1.87 (dd, J=12.3, 6.8, 1H), 1.43-1.34 (m, 5H), 1.27-1.18 (m, 1H), 0.81 (t, J=7.4, 3H). Indoline SI-63: ¹H NMR (500 MHz, CDCl₃, minor product): 6.67-6.64 (m, 2H), 6.27 (d, J=9.1, 1H), 5.04 (s, 1H), 3.75 (s, 3H), 3.63-3.56 (m, 1H), 2.88 (s, 3H), 2.19 (dd, J=11.7, 4.3, 1H), 1.69-1.62 (m, 2H), 1.51-1.43 (m, 5H), 0.86 (t, J=7.4, 3H).

### C. Protecting Group Removal

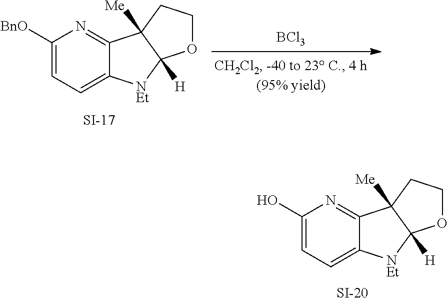

Representative Procedure A (Azaindoline SI-20 is used as an example). Azaindoline SI-20. A 1-dram vial was charged with a magnetic stir bar, flame-dried under reduced pressure, and allowed to cool under a N₂ atmosphere. Azaindoline SI-17 (10.4 mg, 0.033 mmol, 1.0 equiv) was added and the vial was flushed with N2 for 5 min. CH₂Cl₂ (670 uL, 0.05 M) was added and the reaction mixture cooled to −40° C. BCl₃ (1 M in CH₂Cl₂, 235.0 uL, 0.235 mmol, 7.0 equiv) was added dropwise over 1 min and the reaction mixture was stirred at −40° C. After 5 min, the reaction mixture was warmed to 23° C. and allowed to stir for 4 h. The reaction mixture was then transferred to separatory funnel containing ice-cold deionized H₂O (5 mL). The reaction mixture was basified with saturated aqueous NaHCO₃ (5 mL) to a pH of 9 and extracted with EtOAc (5×3 mL). The combined organic layers were washed with saturated aqueous NaCl (10 mL), and dried over Na₂SO₄. The volatiles were removed under reduced pressure, and the crude residue was purified by preparative thin-layer chromatography (EtOAc) to yield azaindoline SI-20 (7.0 mg, 95% yield) as a brown solid. Azaindoline SI-20: ¹H NMR (500 MHz, CDCl₃): δ 6.78 (d, J=8.5, 1H), 6.48 (d, J=8.5, 1H), 5.12 (s, 1H), 3.95 (ddd, J=9.0, 7.4, 1.5, 1H), 3.48 (ddd, J=11.2, 8.9, 5.1, 1H), 3.28 (app. dq, J=14.1, 7.1, 2H), 2.41 (ddd, J=12.4, 5.1, 1.5, 1H), 2.07 (ddd, J=12.4, 11.2, 7.4, 1H), 1.52 (s, 3H), 1.22 (t, J=7.1, 3H).

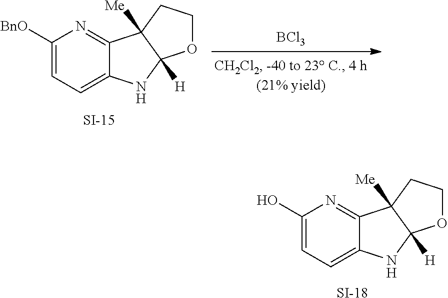

Azaindoline SI-18. Purification by preparative thin-layer chromatography (EtOAc, 2% Et₃N) yielded azaindoline SI-18 (21% yield) as a brown solid. Azaindoline SI-18: ¹H NMR (500 MHz, CDCl₃): δ 7.03 (d, J=8.5, 1H), 6.44 (d, J=8.5, 1H), 5.23 (s, 1H), 3.99 (ddd, J=9.0, 7.3, 1.6, 1H), 3.60 (ddd, J=11.1, 9.1, 5.4, 1H), 2.42 (ddd, J=12.2, 5.4, 1.6, 1H), 2.09 (ddd, J=12.2, 11.1, 7.3, 1H), 1.54 (s, 3H).

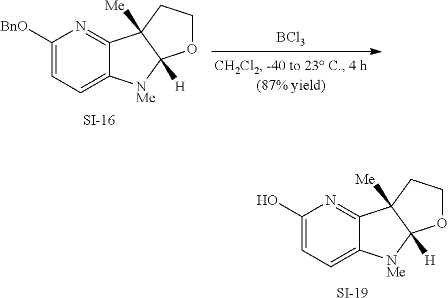

Azaindoline SI-19. Purification by preparative thin-layer chromatography (EtOAc) yielded azaindoline SI-19 (87% yield) as a brown solid. Azaindoline SI-19: ¹H NMR (500 MHz, CDCl₃): δ 6.77 (d, J=8.5, 1H), 6.49 (d, J=8.5, 1H), 5.01 (s, 1H), 3.97 (ddd, J=8.8, 7.4, 1.6, 1H), 3.47 (ddd, J=11.2, 8.8, 5.2, 1H), 2.88 (s, 3H), 2.44 (ddd, J=12.2, 5.2, 1.6, 1H), 2.08 (ddd, J=12.2, 11.2, 7.4, 1H), 1.53 (s, 3H).

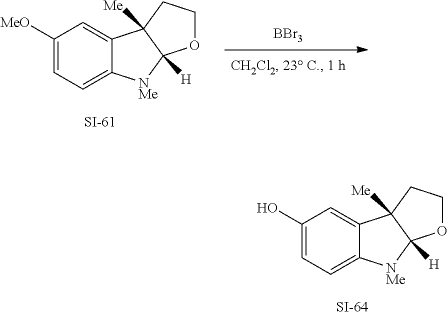

Representative Procedure B (Indoline SI-64 is used as an example). Indoline SI-64. A 1-dram vial was charged with a magnetic stir bar, flame-dried under reduced pressure, and allowed to cool under a N₂ atmosphere. Indoline SI-61 (20.0 mg, 0.091 mmol, 1.0 equiv) was added and the vial was flushed with N₂ for 5 min. CH₂Cl₂ (1.0 mL, 0.08 M) was added and the reaction mixture was left to run at 23° C. BBr₃ (1.0 M in CH₂Cl₂, 460.0 uL, 0.456 mmol, 5.0 equiv) was added dropwise over 1 min and the reaction was left to run at 23° C. for 1 h. After the allotted time, the volatiles were removed under N₂. MeOH (2.0 mL) was then added to the vial and the reaction mixture was allowed to stir for 5 min. The reaction was then concentrated under reduced pressure and the resulting residue was suspended in deionized water (3 mL). The suspension was then transferred to a separatory funnel with EtOAc (2 mL). A saturated aqueous solution of NaHCO₃ (5 mL) was added and the layers separated. The aq. layer was then extracted with EtOAc (3×5 mL). The combined organic layers were washed with saturated aqueous NaCl (5 mL) and dried over Na₂SO₄. The volatiles were then removed under reduced pressure, and the crude residue was taken onto the next step.

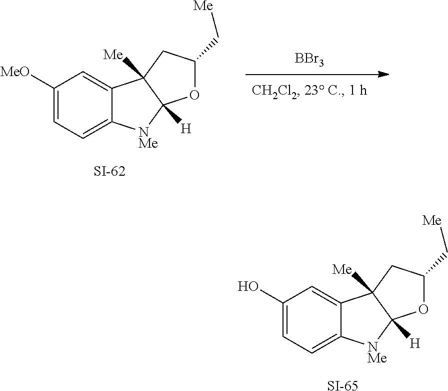

Indoline SI-65. Material taken forward crude to the next reaction.

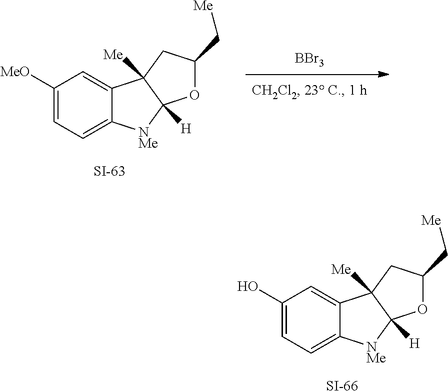

Indoline SI-66. Material taken forward crude to the next reaction.

### D. Carbamoylation

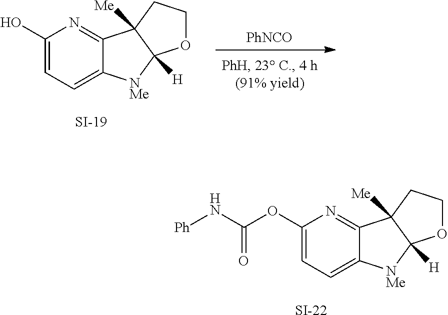

Representative Procedure A (Aza-phensvenine SI-22 is used as an example). Aza-phensvenine SI-22. A 1-dram vial was charged with a magnetic stir bar, flame-dried under reduced pressure, and allowed to cool under a N₂ atmosphere. Azaindoline SI-19 (5.0 mg, 0.0242 mmol, 1.0 equiv) was added and the vial was flushed with N₂ for 5 min. The vial was then charged with a stock solution of PhNCO (1.7 uL, 0.0219 mmol, 1.5 equiv) in PhH (200 uL, 0.075 M), and the reaction mixture was allowed to stir at 23° C. under an N₂ atmosphere. After 4 h, the volatiles were removed under reduced pressure to yield aza-phensvenine SI-22 (7.2 mg, 91% yield) as an amorphous solid. Aza-phensvenine SI-22: ¹H NMR (500 MHz, C₆D₆): δ 7.27 (d, J=8.0, 2H), 7.05-7.02 (m, 2H), 6.83-6.80 (m, 1H), 6.77 (d, J=8.1, 1H), 6.61 (br. s, 1H), 6.13 (d, J=8.1, 1H), 4.86 (s, 1H), 3.65 (ddd, J=8.9, 7.6, 1.6, 1H), 3.25 (ddd, J=11.2, 8.9, 5.4, 1H), 2.52 (s, 3H), 2.26 (ddd, J=12.2, 5.4, 1.6, 1H), 1.67 (ddd, J=12.2, 11.2, 7.6, 1H), 1.32 (s, 3H).

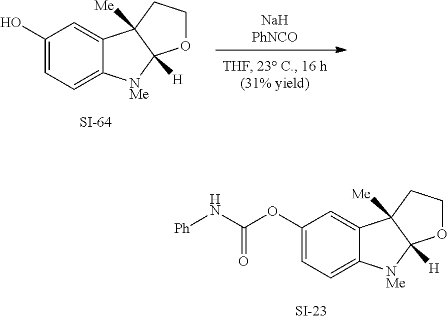

Representative Procedure B (Phensvenine SI-23 is used as an example). Phensvenine SI-23. A 1-dram vial was charged with a magnetic stir bar, flame-dried under reduced pressure, and allowed to cool under a N₂ atmosphere. Indoline SI-64 (18.7 mg, 0.0911 mmol, 1.0 equiv) was added and the vial was flushed with N₂ for 5 min. The material was then dissolved in THF (500 uL, 0.2 M) followed by the addition of NaH (60% dispersion in mineral oil, 1.1 mg, 0.0273 mmol, 0.3 equiv) in one portion under a constant flow of N₂. PhNCO (11.9 mg, 11.0 uL, 0.100 mmol, 1.1 equiv) was then added dropwise over 1 min and the reaction was left to run at 23° C. for 16. After the allotted time, the reaction was quenched by addition of a saturated aqueous solution of NaHCO₃ (5 mL) and transferred to a separatory funnel with EtOAc (5 mL). The layers were separated and the aq. layer was then extracted with EtOAc (3×5 mL). The combined organic layers were washed with saturated aqueous NaCl (5 mL) and dried over Na₂SO₄. The volatiles were then removed under reduced pressure, and the crude residue was purified by preparative thin-layer chromatography (1:1 Hexanes:EtOAC, 2% Et₃N) to yield phensvenine SI-23 (9.2 mg, 31% yield) as a brown solid. Phensvenine SI-23: ¹H NMR (500 MHz, CDCl₃): δ 7.43 (d, J=7.9, 2H), 7.32 (t, J=7.9, 2H), 7.09 (t, J=7.9, 1H), 6.90-6.88 (m, 2H), 6.32-6.30 (m, 1H), 5.09 (s, 1H), 3.96 (ddd, J=8.7, 7.5, 1.4, 1H), 3.50 (ddd, J=11.1, 8.7, 5.2, 1H), 2.91 (s, 3H), 2.13 (ddd, J=12.1, 5.2, 1.3, 1H), 2.04 (app. ddd, J=12.1, 11.1, 7.5, 1H), 1.46 (s, 3H). Compounds SI-33-SI-40 can be synthesized using this general approach.

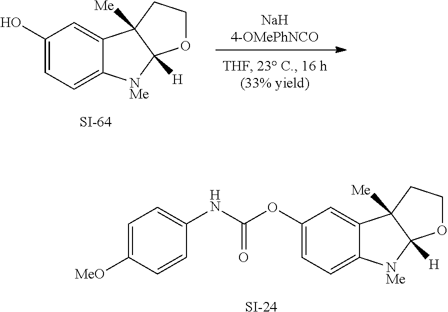

Indoline SI-24. Purification by preparative thin-layer chromatography (1:1 Hexanes:EtOAc, 2% Et₃N) yielded indoline SI-24 (33% yield) as a light brown solid. Indoline SI-24: ¹H NMR (500 MHz, CDCl₃): δ 7.34 (br. d, J=7.9, 2H), 6.89-6.86 (m, 4H), 6.32-6.29 (m, 1H), 5.08 (s, 1H), 3.95 (ddd, J=8.7, 7.5, 1.6, 1H), 3.79 (s, 3H), 3.49 (ddd, J=11.1, 8.7, 5.6, 1H), 2.9 (s, 3H), 2.12 (ddd, J=12.1, 5.6, 1.6, 1H), 2.03 (app. ddd, J=12.1, 11.1, 7.5, 1H), 1.46 (s, 3H).

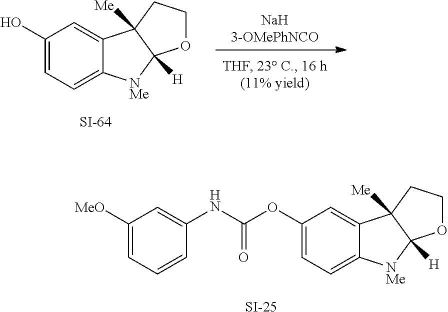

Indoline SI-25. Purification by preparative thin-layer chromatography (1:1 Hexanes:EtOAc, 2% Et₃N) yielded indoline SI-25 (11% yield) as a light brown solid. Indoline SI-25: ¹H NMR (500 MHz, CDCl₃): δ 7.21 (t, J=8.0, 2H), 6.90-6.86 (m, 3H), 6.64 (ddd, J=8.2, 2.4, 0.7, 1H), 6.31 (d, J=8.2, 1H), 5.08 (s, 1H), 3.95 (ddd, J=8.7, 7.3, 1.6, 1H), 3.79 (s, 3H), 3.49 (ddd, J=11.1, 8.7, 5.3, 1H), 2.90 (s, 3H), 2.13 (ddd, J=12.1, 5.4, 1.6, 1H), 2.03 (app. ddd, J=12.1, 11.1, 7.3, 1H), 1.46 (s, 3H).

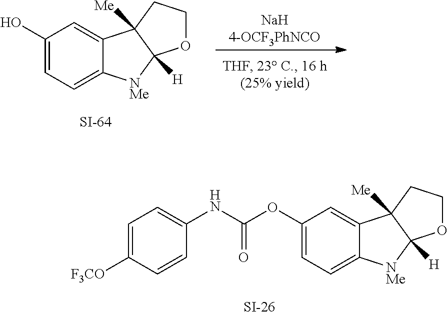

Indoline SI-26. Purification by preparative thin-layer chromatography (1:1 Hexanes:EtOAc, 2% Et₃N) yielded indoline SI-26 (11% yield) as a white solid. Indoline SI-26: ¹H NMR (500 MHz, CDCl₃): δ 7.46 (d, J=8.0, 2H), 7.18-7.17 (m, 2H), 6.89-6.86 (m, 2H), 6.32-6.31 (m, 1H), 5.09 (s, 1H), 3.96 (ddd, J=8.7, 7.3, 1.5, 1H), 3.49 (ddd, J=11.1, 8.7, 5.2, 1H), 2.90 (s, 3H), 2.12 (ddd, J=12.1, 5.2, 1.5, 1H), 2.04 (app. ddd, J=12.1, 11.1, 7.5, 1H), 1.45 (s, 3H).

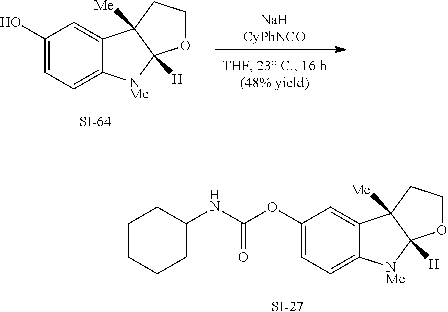

Indoline SI-27. Purification by preparative thin-layer chromatography (2:1 Hexanes:EtOAc) yielded indoline SI-27 (11% yield) as an off-white solid. Indoline SI-27: ¹H NMR (500 MHz, CDCl₃): δ 6.82-6.80 (m, 2H), 6.27 (d, J=8.2, 1H), 5.06 (s, 1H), 3.93 (ddd, J=8.8, 7.5, 1.4, 1H), 3.48 (ddd, J=11.1, 8.8, 5.5, 1H), 2.89 (s, 3H), 2.12 (ddd, J=12.1, 5.5, 1.4, 1H), 2.05-1.98 (m, 3H), 1.74-1.71 (m, 2H), 1.63-1.60 (m, 2H), 1.44 (s, 3H), 1.38-1.32 (m, 2H), 1.26-1.17 (m, 3H).

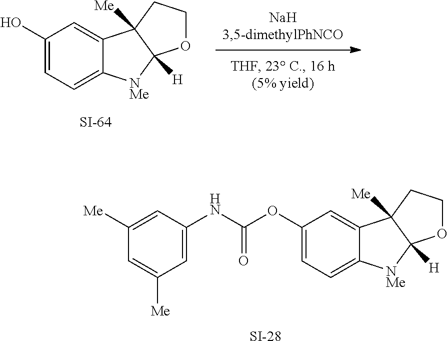

Indoline SI-28. Purification by preparative thin-layer chromatography (1:1 Hexanes:EtOAc) yielded indoline SI-28 (5% yield) as a light brown solid. Indoline SI-28: ¹H NMR (500 MHz, CDCl₃): δ 6.99-6.98 (m, 3H), 6.88-6.86 (m, 2H), 6.31-6.29 (m, 1H), 5.08 (s, 1H), 3.94 (ddd, J=8.7, 7.4, 1.5, 1H), 3.49 (ddd, J=11.1, 8.7, 5.3, 1H), 2.90 (s, 3H), 2.23 (app. s, 6H), 1.45 (s, 3H).

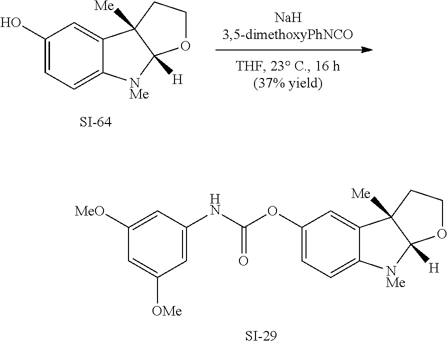

Indoline SI-29. Purification by preparative thin-layer chromatography (3:1 PhH:Et₂O) yielded indoline SI-29 (37% yield) as a colorless oil. Indoline SI-29: ¹H NMR (500 MHz, CDCl₃): δ 6.91-6.86 (m, 3H), 6.67 (s, 2H), 6.31 (d, J=8.3), 6.22 (t, J=2.2, 1H), 5.09 (s, 1H), 3.97-3.94 (m, 1H), 3.77 (s, 6H), 3.49 (ddd, J=11.1, 8.8, 5.5, 1H), 2.90 (s, 3H), 2.14-2.11 (m, 1H), 2.07-2.01 (m, 1H), 1.46 (s, 3H).

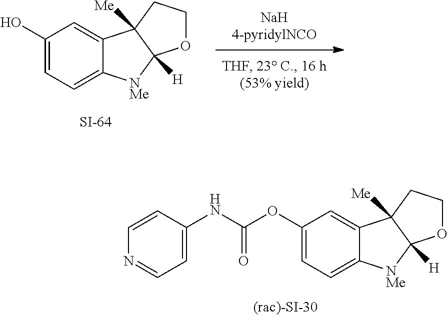

Indoline (rac)-SI-30. Purification by preparative thin-layer chromatography (1:1 PhH:EtOAc) yielded indoline (rac)-SI-30 (53% yield) as a white solid. Indoline (rac)-SI-30: ¹H NMR (500 MHz, CDCl₃): δ 8.50 (d, J=6.6, 2H), 7.38-7.37 (m, 2H), 6.88-6.86 (m, 2H), 6.31 (d, J=8.3, 1H), 5.10 (s, 1H), 3.96 (ddd, J=8.8, 7.3, 1.5, 1H), 3.49 (ddd, J=11.1, 8.8, 5.4, 1H), 2.91 (s, 3H), 2.13 (ddd, J=12.1, 5.4, 1.5, 1H), 2.04 (app. ddd J=12.1, 11.1, 7.3, 1H), 1.46 (s, 3H).

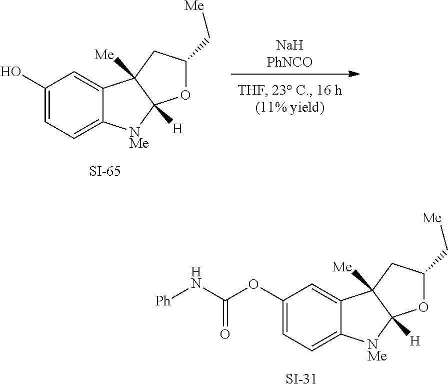

Indoline SI-31. Purification by preparative thin-layer chromatography (15:1 PhH:EtOAc) yielded indoline SI-31 (11% yield) as a white solid. Indoline SI-31: ¹H NMR (500 MHz, CDCl₃): δ 7.44 (app. d, J=8.0, 2H), 7.34-7.31 (m, 2H), 7.11-7.08 (m, 1H), 6.90-6.87 (m, 2H), 6.34 (d, J=8.4, 1H), 4.99 (s, 1H), 4.00 (pent., J=6.6, 1H), 2.92 (s, 3H), 2.15 (dd, J=12.1, 6.5, 1H), 1.87 (dd, J=12.1, 6.5, 1H), 1.46-1.40 (m, 1H), 1.38 (s, 3H), 1.29-1.24 (m, 2H), 0.83 (t, J=7.4, 3H).

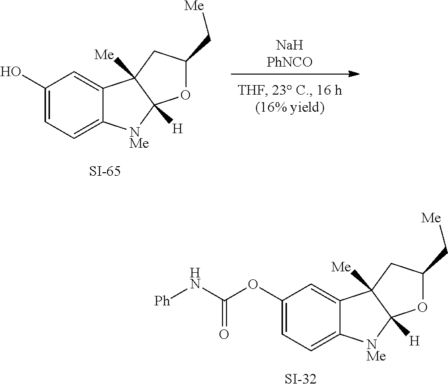

Indoline SI-32. Purification by preparative thin-layer chromatography (4:1 Hexanes:EtOAc) yielded indoline SI-32 (16% yield) as a light brown solid. Indoline SI-32: ¹H NMR (500 MHz, CDCl₃): δ 7.44 (app. d, J=8.2, 2H), 7.33 (app. t, J=7.3, 2H), 7.11-7.08 (m, 1H), 6.88-6.86 (m, 2H), 6.30 (d, J=8.2, 1H), 5.09 (s, 1H), 3.66-3.60 (m, 1H), 2.91 (s, 3H), 2.19 (dd, J=11.9, 4.6, 1H), 1.68-1.58 (m, 3H), 1.51-1.37 (m, 4H), 0.87 (t, J=7.36, 3H).

### E. Palladium-Catalyzed Cross Coupling

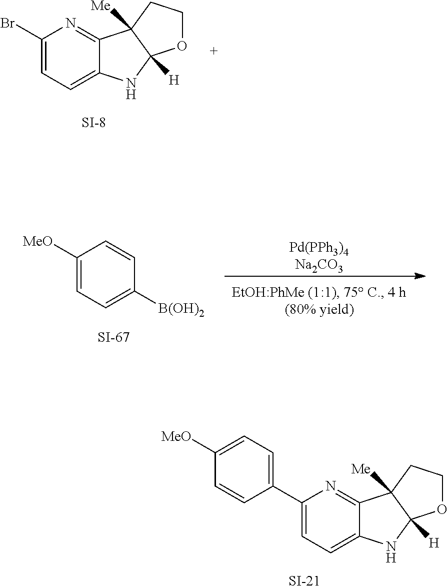

Azaindoline SI-21. A 1-dram vial was charged with a magnetic stir bar, flame-dried under reduced pressure, and allowed to cool under a N₂ atmosphere. Azaindoline SI-8 (13.0 mg, 0.048 mmol, 1.0 equiv), (4-methoxyphenyl)boronic acid SI-67 (8.8 mg, 0.058 mmol, 1.2 equiv), and Pd(PPh₃)₄ (1.9 mg, 0.002 mmol, 3.5 mol %) was added and the vial was flushed with N2 for 5 min. Toluene (320 uL, 0.15 M), EtOH (320 uL, 0.15 M), and a 2.0 M aqueous solution of Na₂CO₃ (480 uL, 0.1 M) was then added to the vial. The mixture was then sparged with N₂ for 5 min. The vial was then capped with a Teflon-lined screw cap and placed in a pre-heated aluminum block and allowed to stir at 75° C. for 4 h. After the allotted time, the reaction was allowed to cool to 23° C. over 20 min. The reaction was then diluted with EtOAc (5 mL) and deionized water (3 mL) and transferred to a separatory funnel. The layers were separated and the aq. layer extracted with EtOAc (3×5 mL). The combined organic layers were washed with saturated aqueous NaCl (5 mL), and dried over Na₂SO₄. The volatiles were removed under reduced pressure, and the crude residue was purified by preparative thin-layer chromatography (CH₂Cl₂) to yield azaindoline SI-21 (80% yield) as a colorless oil. Azaindoline SI-21: ¹H NMR (500 MHz, CDCl₃): δ 7.87-7.86 (m, 2H), 7.31 (d, J=8.2, 1H), 6.96-6.94 (m, 2H), 6.83 (d, J=8.2, 1H), 5.34 (s, 1H), 4.61 (br. s, 1H), 3.98 (ddd, J=8.9, 7.5, 1.6, 1H), 3.84 (s, 3H), 3.55 (ddd, J=11.1, 8.9, 5.3, 1H), 2.54-2.50 (m, 1H), 2.13-2.07 (m, 1H), 1.55 (s, 3H).

### F. Separation of Enantiomers

Compound (rac)-SI-30 was separated into its enantiomers on a 100 mg scale. Absolute configuration was not determined.

| Preparative method: | Analytical Method: |
|---|---|
| OD-H (10 × 250 mm) | OD-H (4.6 × 250 mm) |
| 15% isopropanol with | 25% isopropanol with |
| 0.2% Et₃N in CO₂, 100 bar | 0.2% Et₃N in CO₂, 100 bar |
| 30 mL/min | 3.5 mL/min |

### Example 4: Exemplary nSMase2 Dose Response Assay

To evaluate nSMase2 inhibitory activity of tested compounds, cell lysates from HEK293T cells over-expressing human nSMase2 were used as the source of the nSMase2 enzyme. The Amplex Red Sphingomyelinase activity assay was performed according to previously a described protocol¹. Confluent cells were washed with PBS and harvested with lysis buffer [100 mM Tris-HCl pH 7.5, 100 mM sucrose, 100 μM PMSF, 1× protease inhibitor cocktail] using a cell scraper, and then sonicated 3 times on ice for 10 sec and centrifuged at 10,000×g for 10 min at 4° C. to remove remaining cell debris. An initial screening of a compound library was performed using 50 μM compound concentrations. In secondary testing, selected compounds were analyzed at multiple compound concentrations and plotted as a percentage of DMSO control; dose-response curves were generated to determine IC₅₀s using GraphPad Prism software (GraphPad Software, La Jolla Calif.). In order to determine the mechanism of nSMase2 inhibition by 8 and 11, enzyme kinetic analysis was performed as follows: nSMase2 activity assay was performed with a range of the substrate sphingomyelin; concentrations ranged from 10 to 400 μM in the presence of 4 different concentrations of the inhibitors—0.1, 0.5, 1, and 5 μM. Activity of the enzyme in the presence of the different concentrations of the inhibitors was plotted against substrate concentration and the maximal rate of the enzymatic reaction (Vₘₐₓ) and Michaelis constant were calculated using GraphPad Prism.

### Example 5: Exemplary nSMase2 Docking Analysis

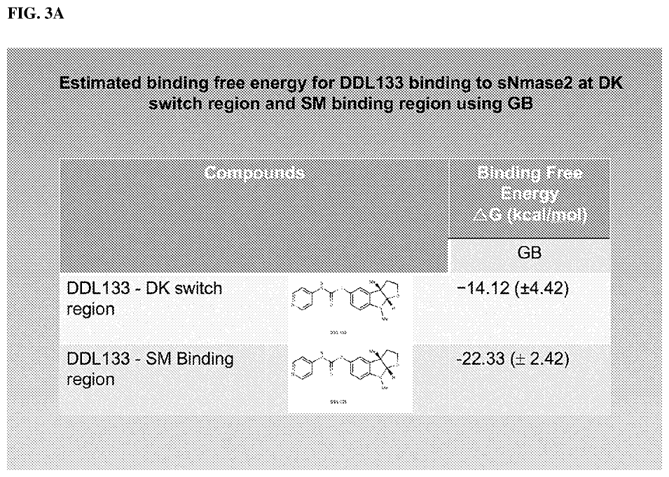

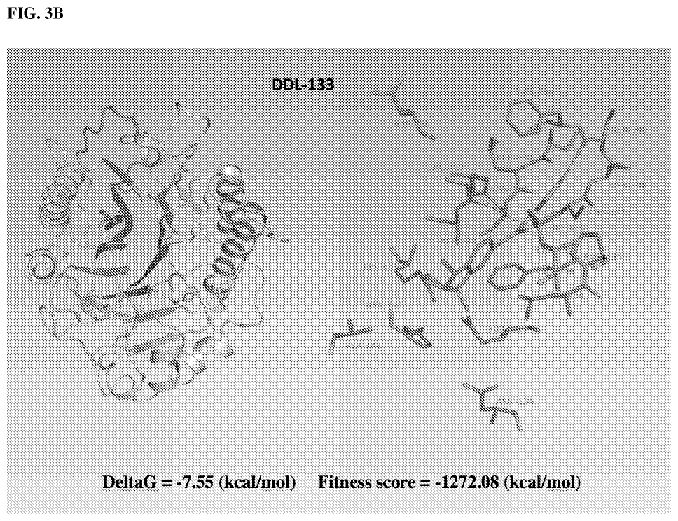

Molecular docking analysis of compound 8 to nSMase2 (pdp: 5UVG) was performed using the Swiss Dock server on a Area51 Work Area 51 R4 linux workstation configured with one intel core i7 @ 3.5 Gz Hexa core processor, 32 GB RAM and NVIDIA GTX 1080 Ti with 11 GB GDDR5X GPU card to carry out molecular modeling, docking, molecular dynamics as well as X-ray data processing, model building and structure refinement processing. Prior to docking, missing regions in the nSMase2 crystal structure were built using the MODELER program. All rotatable single bonds were allowed to rotate in compound cambinol and the docking results were screened and analyzed with the Chimera program. Molecular Dynamics (MD) simulation was performed to determine the binding free energy of compound cambinol binding to nSMase2. An AMBER16 package was used to perform the MD simulation. The Antechamber module in AMBER was used to generate the parameters for compound 8. The SwissDock web server was used to predict compound cambinol binding to nSMase2. The results of this docking analysis are depicted in FIG. 3.

### Example 6: Exemplary AChE Dose Response Assay for Certain Compounds of the Disclosure

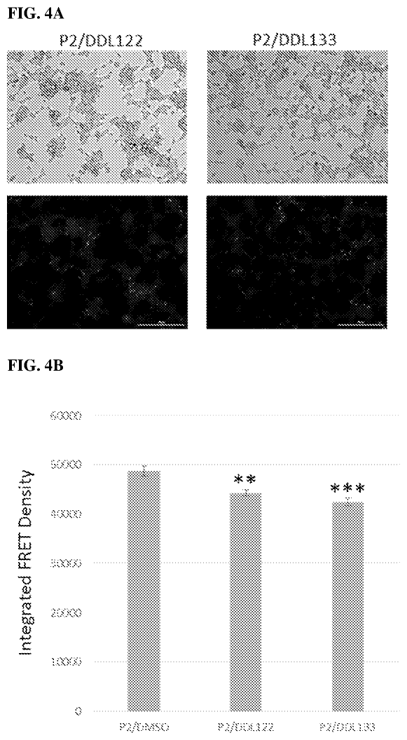

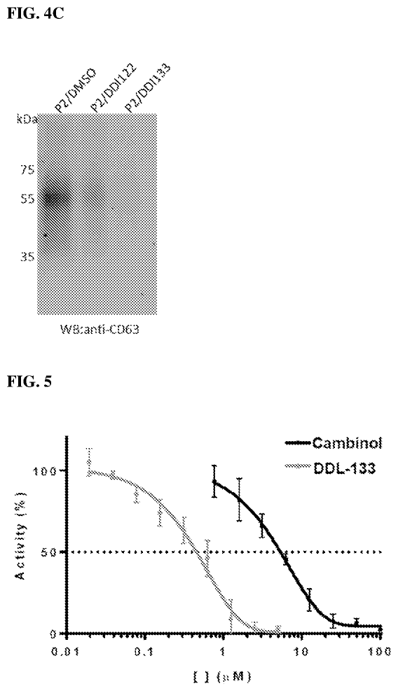

This acetylcholinesterase (AChE) assay is based on an Amplex Red assay kit (Thermo Fisher A12217). Human AChE (Sigma) was used. Briefly, 5 μL of compounds at the desired concentration were loaded into wells of a 384-well plate. Then, 5 μl of human AChE (30 mU/mL) was loaded into each well, followed by the addition of 10 μL of the working solution which contains 400 μM Amplex Red reagent, 2 U/mL HRP, 0.2 U/mL choline oxidase, and 100 μM acetylcholine. The reaction was monitored for 60 minutes and read at 530/590 nm. AChE IC₅₀ was determined by plotting AChE activity as a percentage of DMSO control using GraphPad Prism software. The results of this docking analysis are depicted in FIG. 4B.

### Example 7: Mechanism of nSMase2 Inhibition by Fluoroindoline Compounds

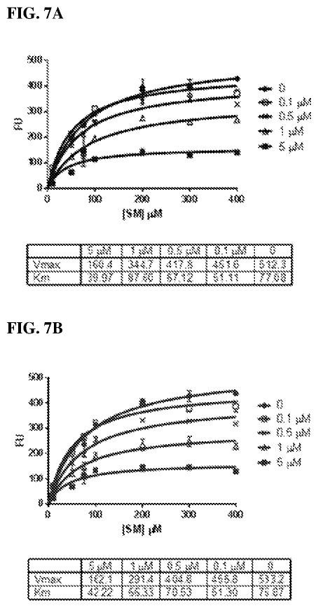

To determine the type of nSMase2 inhibition by compounds 8 and 11, kinetics assays were performed. As shown in FIGS. 7A and 7B, increasing concentrations of compounds 8 and 11 resulted in decreasing Kₘ values as well as concomitant decreases in the Vₘₐₓ, indicative of a non-competitive mechanism of inhibition of nSMase2. Thus, it can be concluded that both compounds bind to the enzyme and inhibit enzyme-substrate complex formation.

Using a published crystal structure of the nSMase2 catalytic domain, it was found that both 8 and 11 could bind to nSMase2 in the distal DK-switch (Asp-Lys) site located away from the substrate sphingomyelin site and in concordance with the kinetic analysis as modulation of the DK-switch could lead to non-competitive inhibition of the enzyme. This aligns with previously published studies on the known nSMase2 inhibitor cambinol that was also shown to bind the nSMase2 catalytic domain in the region of the DK switch and prevent enzyme activation by keeping the switch in the ‘off’ position. Molecular Dynamics (MD) simulation was performed to determine the binding free energy of compound 8 binding to nSMase2. Compound 8 stays at the DK-switch site of nSMase2 through the 50 ns simulation with an estimated binding energy of −14.3 kcal/mol.

### Example 8: Exemplary In Vitro Inhibition of Tau Propagation by Certain Compounds of the Disclosure

Brain autopsy samples were obtained from the University of California Irvine, University of Southern California AD Research Centers, and University of California, Los Angeles. Brain tissue was cryopreserved and synaptosomal fractions (P2-fractions, or P2) were prepared as previously described. In order to prepare P2-extracts, aliquots were quickly defrosted at 37° C. and centrifuged at 10,000×g for 10 min at 4° C. to remove P2 from sucrose. After aspirating the supernatant, cold PBS was added to each sample in Ma 1:5 weight/volume ratio. Samples were then sonicated in 10-second intervals three times, incubated on ice for 30 min and centrifuged at 20,000 g for 20 min at 4° C. P2 extracts were collected and stored at −80° C. Tau biosensor cell treatment and analysis. HEK293T Tau RD P301S FRET biosensor cells (tau biosensors) were growing in the Dulbecco's Modified Eagle's medium (DMEM) with higher glucose, 10% FBS, and 1% Penicillin-streptomycin at 37° C./5% CO2. Cells were plated in 10-sm dishes (3 million cells per dish in the regular medium) and grown for 12 hrs. Cells were transduced with pulled synaptosomal (P2) extracts from AD cases using lipofectamine 2000. 35 μg of pulled AD material per one 10-sm dish was used. Defrosted P2 extracts were sonicated in water bath sonicator for 10 minutes, and diluted with Opti-MEM serum reduced medium to the final volume 200 μL per 10-sm dish. In a separate tube lipofectamine 2000 was combined with Opti-MEM medium, based on 25 μl of lipofectamine and 175 μL OptiMEM medium per each 10-sm dish, and incubated for 10 minutes at room temperature (RT). Each P2 extract mix (200 μl) and prepared lipofectamine 2000 mix (200 μl) was combined and incubated for 20 min at RT. Each 10-sm dish with cell received 400 μl of the final liposomes in the total volume of medium 5 ml per dish. Cultures treated with equal amount of empty liposomes were used as a control. Cells were incubated with liposomes for 20 hrs and then trypsinized/washed to eliminate exogenous tau seeds and re-plated in exosome-free medium (30,000 cells per well of 96-well plate) with either 10 μM of DDL-112 (cambinol), DDL-122, DDL-133 or DMSO (0.001%) for control.

Cells were imaged after 24 hrs using automated microscope imaging system (Lionheart FX) equipped with 20× objective and CFP-YFP FRET Image filter cube. Treatment with the nSMase2 inhibitors (DDL-112 and 133) visibly decreased amount of FRET-positive tau aggregates comparing to DMSO-treated control (FIG. 5A). Moreover decrease in the FRET-positive signal is in agreement with nSMase2 activity of the inhibitors (DDL-133>DDL-122). 60 hrs after the start of the treatment, cells and cell medium were collected. Cells were prepared for flow cytometry as previously described. FRET signal was detected using LSRII flow cytometer (BD biosciences). Cells were backgated onto forward scatter versus side scatter to insure a single cell analysis, 5,000 cells were collected within the gate. FRET negative and FRET-positive cell populations were defined as previously described. Flow cytometry data presented in FIG. 5B confirmed our microscopy data described above and demonstrated significant decrease in FRET-positive signal (integrated FRET density=% of FRET pos cells×Median of FRET intensity) for inhibition of nSMase2 activity in cells with DDL133>DDL122.

Extracellular vesicles (EVs) were purified from the cell culture medium using Exo-Quick-TC exosome purification kit (SBI, EXOTC10A-1) according to the manufacturing instructions. Immunoblot analysis of the EV fractions was done by 10-20% Tris-Glycine gel in non-reduced conditions, transferred to PVDF membrane and probed with antibodies against CD63 (ThermoFisher, 10628D), followed by HRP-conjugated secondary antibodies. Chemiluminescent signals were obtained with Super Signal West Femto substrate (Thermo Scientific Pierce 34,095) and detected using a BioSpectrum 600 imaging system and quantified using VisionWorks Version 6.6A software (UVP; Upland, Calif.). A gradual decrease in the level of expression of exosomal marker CD63 (DMSO>DDL122>DDL133, FIG. 5C) was observed. This reflects strong inhibition of extracellular vesicle release by the new class of dual nSMase2/AChE inhibitors.

Exosome production and uptake are part of normal cell physiology. Although molecular pathways may vary between cell types and depend on cell homeostasis, most cells communicate through exosomal exchange both in vivo and in vitro. Using known nSMase2 inhibitors cambinol and GW4869, the role of the nSMase2-dependent pathway of EV biogenesis in tau transmission from donor to recipient cells in this non-neuronal cell model has been demonstrated using two different in vitro assays—the Donor plus Recipient (D+R) assay and EV-mediated transfer (EMT) assay.

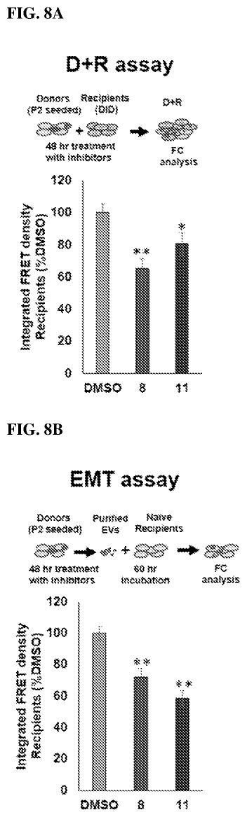

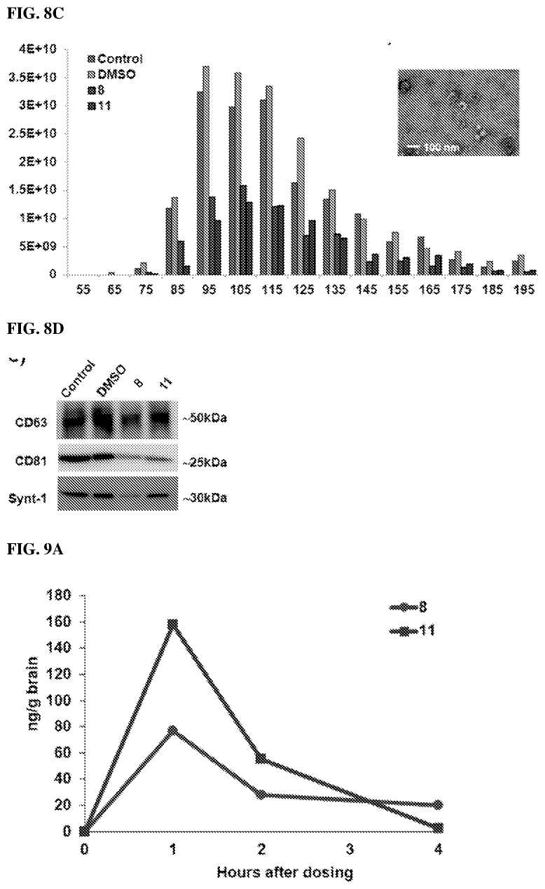

The principles of D+R and EMT assays are presented in schematic form in FIGS. 8A and B, respectively. Our data demonstrates that treatment with 8 or 11 at a concentration of 20 μM significantly suppresses tau seed transfer from donor to recipient cells in the D+R and EMT assays. Shuttling by tau-bearing EVs is not the only pathway of tau seed transfer between cells in vivo or when donor and recipient cells are growing together in vitro, as in the D+R assay. In contrast, the EMT assay lets us isolate the effect of the inhibitors on EV-mediated tau seed transmission, which can explain the profound difference in the magnitude of FRET fluorescence density by dual nSMase2/AChE inhibitor 11 between the assays—19.5% decrease from dimethyl sulfoxide (DMSO)-control in D+R assay and 41.3% decrease in EMT assay.

The EVs purified from the seeded donor cells growing in the presence of our dual inhibitor compounds or DMSO control were characterized. Successful purification of EVs was confirmed by tunable resistive pulse sensing (TRPS) (FIG. 8C), transmission electron microscopy (TEM) (FIG. 8C), and western blotting analysis with known exosomal markers (FIG. 8D). Treatment with dual nSMase2/AChE inhibitor 8 or 11 did not affect EV size distribution, but decreased the concentrations of exosomal-type small EVs (FIG. 8C). Levels of exosomal markers CD63, CD81, and syntenin-1 were reduced in EVs purified from 8—and 11—treated cells in comparison with the DMSO control (FIG. 8D). Relatively high suppression of tau transfer by 11 compared to 8 in the EMT assay may be related to the greater AChE inhibitory activity of 11 in conjunction with its nSMase2 inhibition and the role of dual inhibitory activity in exosome-mediated transfer of tau seeds.

Cell viability and/or rate of proliferation may have an effect of tau seed transfer from donor to recipient cells through different mechanisms. Thus, the effects of tau seeding and treatment with nSMase2/AChE inhibitors on donor cell number and viability were evaluated. Twenty-four hour exposure to AD human brain synaptosomal extracts decreased the rate of the donor cell survival in the next passage compared to cells treated with lipofectamine 2000. The specific mechanisms of cell death in tau-seeded donor cultures was not determined. It is possible that a subset of tau-bearing EVs inhibited by nSMase2 inhibitors are apoptotic exosome-like vesicles (AEVs) that—in contrast to apoptotic bodies—represent a subtype of exosomes originating from multivesicular endosomes (MVE) at the early apoptotic phase. AEV biogenesis is controlled by the ESCRT-independent sphingosine1-phosphate (S1P)/S1PRs signaling pathway and it can be partially inhibited by nSmase2 inhibitor GW4869 (42). AChE inhibitors are known to protect different cell types, including HEK293T, from apoptosis and thus dual inhibitor 11 could potentially indirectly suppress AEV production. Although it is not possible to exclude this scenario, the treatment of donor cells with 11 for 48 hours after sub-culture didn't affect donor cell numbers or survival compared to DMSO or compound 8 treated donor cells. It was hypothesized that other factors may contribute to the greater effect of 11 on tau seed transfer in the EMT assay. Interestingly, intracellular uptake of tau mediated by the muscarinic acetylcholine receptors (mAChR) M1 and M3 was recently reported. Thus, accumulation of tau oligomers may exacerbate cholinergic deficit in AD through suppression of ACh uptake via mAChR M1/M3 receptors on postsynaptic terminals. Based on similar reasoning, inhibition of AChE could also have a direct effect on tau seed uptake through the increased levels of ACh in the synapse and M1/M3 receptor occupancy.

The preliminary experiments using rivastigmine, a AChE (but not nSMase2) inhibitor, reveals that inhibition of AChE may partially suppress EV-mediated transfer from donor to recipient cells (data not shown) providing further support to our hypothesis. Thus, the dual nSMase2/AChE inhibitors 8 and 11 may simultaneously effect both tau seed release and uptake.

### Example 9: Brain Pharmacokinetics and Target Engagement for Leading Compounds

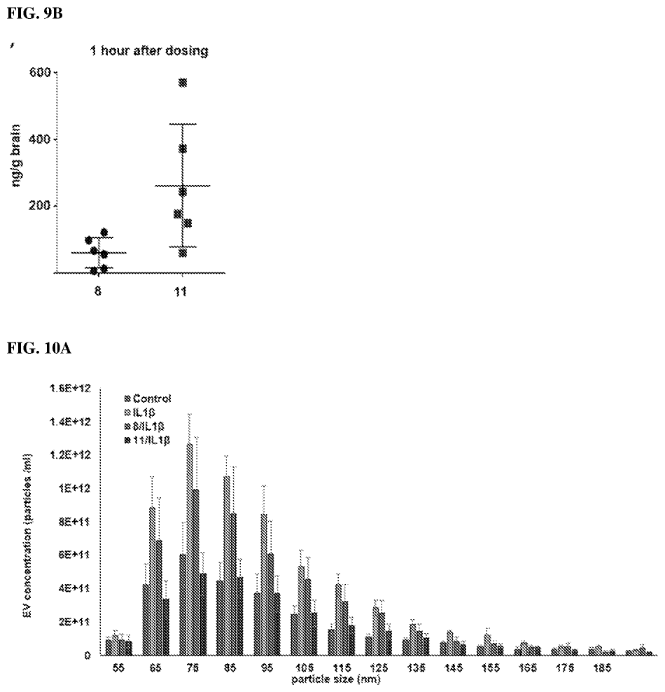

Pharmacokinetic (PK) analysis on the leads 8 and 11 to determine brain permeability using wild type mice was performed. The compounds were subcutaneously (SQ) injected at a dose of 20 mg per kg of body weight (mpk). Brain and plasma samples were collected 1, 2, and 4 hours after dosing. The PK analysis revealed that 8 and 11 reached peak brain levels around one hour after SQ dosing and brain levels were detected for both compounds 2-4 hrs after injection (FIG. 9A).

To carefully evaluate brain compound levels at the Tₘₐₓ (1 hour) time point, 20 mpk SQ dosing of compounds 8 and 11 was performed again using 6 mice per group. Average brain level of the compounds at the peak was equal to 61 ng/g (˜0.2 μM) and 262 ng/g (˜0.8 μM) for compounds 8 and 11 respectively (FIG. 4B). This data confirmed good brain permeability of the lead compounds as was predicted by in silico and PAMPA analysis (Table 2). Compound 11 showed higher average brain levels compared to 8 and the brain levels corresponded to ˜2-fold nSMase2 IC50 by and ˜0.5-fold AChE IC50.

### Example 10: Inhibition of Brain Exosome Release by the Selective nSMase2 and Dual nSMase2/AChE Inhibitors in Rapid In Vivo Assay

The chronic inflammation that is reported in AD and tauopathy models is characterized by elevated levels of pro-inflammatory cytokines in brain parenchyma, including interleukin 1β (IL1β), known to induce nSMase2 activity through the IL1-Receptor 1 (IL1-R1) (44). Neuroinflammation and upregulation of IL1β signaling is linked with an early stage of tauopathy development; blocking of IL1β signaling in the 3×Tg mouse AD model attenuates tau pathology and rescues cognition. It was demonstrated that striatal injection of IL1β to wildtype mice induced release of astrocyte-derived EVs into the blood, resulting in peripheral acute cytokine responses which can be suppressed by pre-treatment with nSMase2 inhibitors.

In order to rapidly test the dual nSMase2 inhibitors in vivo, Tau P301S (PS19 line) tauopathy mouse model was used. For the in vivo assay there were 4 groups: group I (control) received SQ injection of vehicle (DMSO) and intracerebroventricular (ICV) injection of another vehicle (0.0006% BSA in PBS, pH7.4) an hour after SQ treatment; group II (IL1β) received SQ injection of vehicle and unilateral ICV injection of 0.2 ng of IL1β an hour later; group III (8/IL1β)—SQ treatment with 20 mg/kg of 8 and ICV injection of 0.2 ng of IL1β; and group IV—SQ treatment with 20 mg/kg of 11 and ICV injection of 0.2 ng of IL1β. The one-hour interval between treatment with the inhibitors and IL1β ICV injection was chosen based on the brain PK analysis presented above. All animals were sacrificed at 3 hrs after compound or vehicle treatment and 2 hrs after ICV injection of IL1β. Brain EVs were purified as previously described.

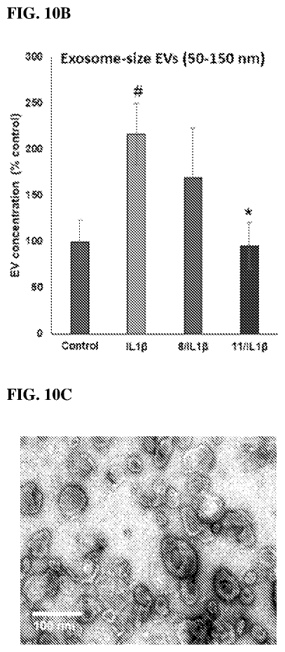

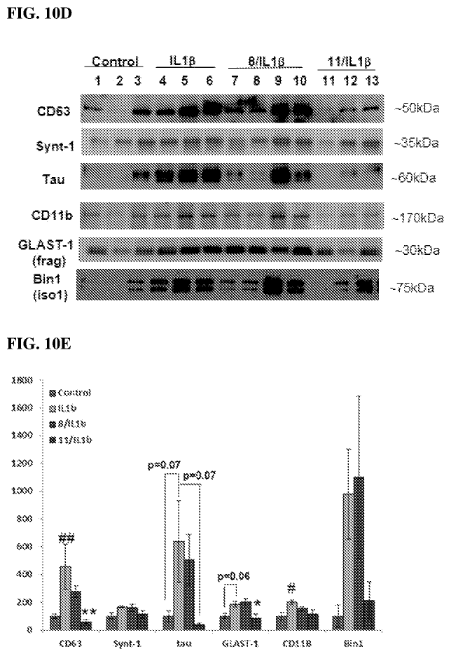

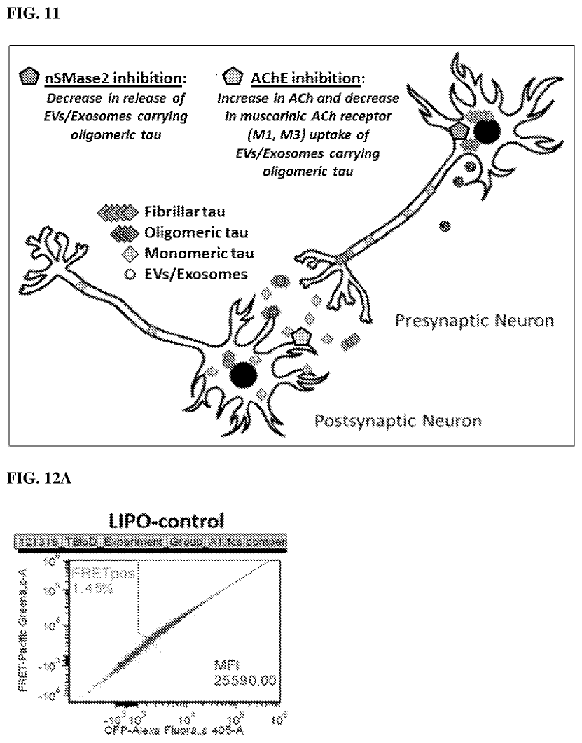

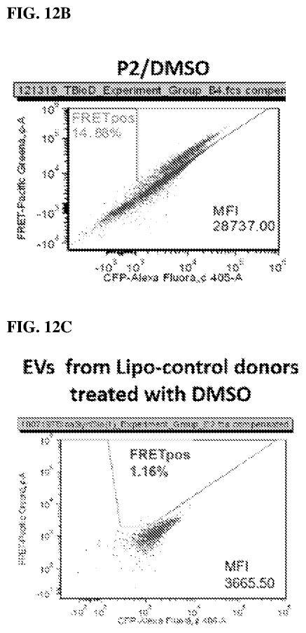

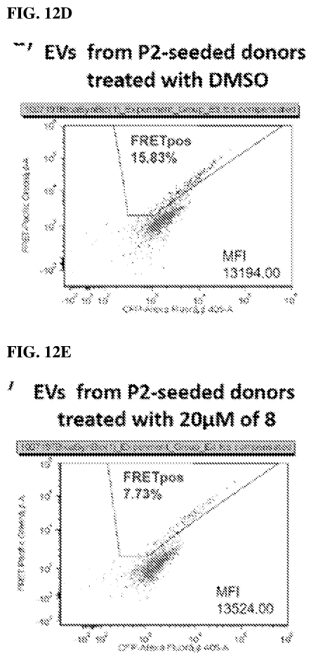

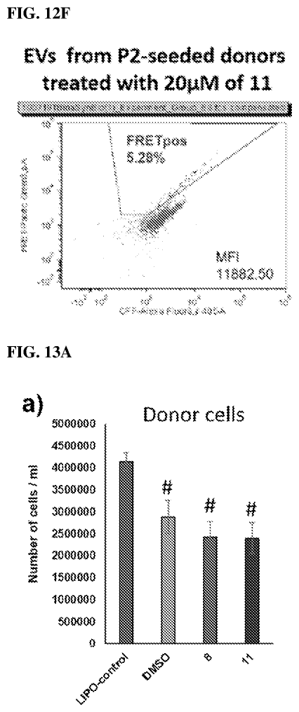

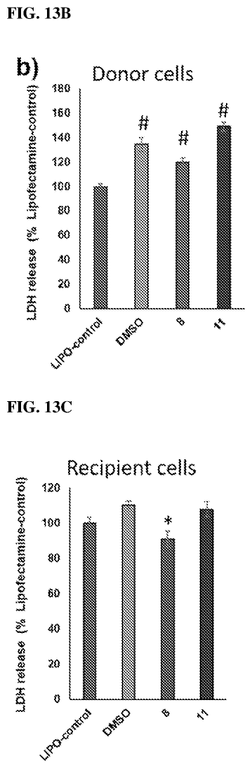

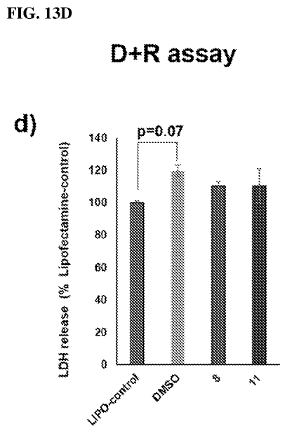

Size distribution and concentration of brain EVs were analyzed using the TRPS method. There were no significant differences in EV size distribution between experimental groups (FIG. 10A). The collected fraction (F2) consists mostly of small exosome-size EVs with a mode equal to 80±5 nm based on TRPS analysis. A high abundance of exosome-sized EVs was confirmed by TEM analysis (FIG. 10C). It was found that ICV injection of IL1β significantly increased the concentration of small EVs (size 50-100 nm) purified from the brain, more than 2 times control (FIG. 10B). Dual nSMase2/AChE inhibitor 11 suppressed IL1β-induced exosomal release to the control level (FIG. 10B), while the less brain-permeable dual inhibitor 8 didn't induce the same level of suppression.

Biochemical analysis of brain-derived EVs (FIGS. 10D & E) showed that pretreatment with lead compound 11 led to a significant reduction of exosomal marker CD63 in exosome-enriched F2 fractions compared to the group treated only with IL1β. In contrast to significant changes in common exosomal marker CD63, levels of syntenin-1, a marker of a specific exosomal subpopulation generated through the Syndecan-Syntenin-ALIX pathway were not different between the groups (FIGS. 10D & E). These results confirm that IL1P stimulation and nSMase2 inhibition have effects on specific populations of exosomes.

The data suggests that the nSMase2-dependent pathway of exosome biogenesis is a part of generation of tau-bearing exosomes in PS19 mice. Tau levels in the F2 fraction showed a strong trend of being elevated in animals treated with IL1β, with the average tau level being around 6 times higher in the IL1β-treated group compared to the control group (FIGS. 10D & 5E). Pretreatment with 11 significantly reduced IL1β-induced tau release by exosomes. The lead compound 8 was less effective in these studies. The known variability of tau load between PS19 mice likely accounts for the lack of statistical significant despite the high magnitude of tau changes.

Multiple brain cell types express IL1-R1, including subpopulations of neurons, astrocytes, choroid plexus cells and ependymal cells, thus the nSMase2-mediated exosomal release by different types of brain cells can be affected differently in response to acute increases in intracerebral IL1β concentration. A couple of cell-type specific markers were used to assess the origin of the IL1β/nSMase2 sensitive exosomal population. It was found that levels of astrocytic glutamate-aspartate transporter (GLAST) and microglial marker CD11b were significantly elevated in F2 fractions isolated from IL1β-treated animals. GLAST is known to be sensitive to papain and the enzyme used for gentle brain tissue dissociation, therefore a 30 kDa fragment of GLAST instead of full-length protein was used for the analysis. Pretreatment with the dual nSMase2/AChE inhibitor 11 significantly reduced the level of astrocyte-derived exosomes and showed the same trend for microglia-derived exosomes, but the difference in CD11b levels between IL1β and 11/IL1β groups was not significant (FIGS. 10D & E). This finding correlates with previously demonstrated IL1β-induced nSMase2-mediated production of astrocyte-derived exosomes in wild type mice. Microglia play an important role in tau spread and inhibition of microglial nSMase2-dependent exosome release suppresses tau propagation in mouse models. The low levels of microglia response in our rapid in vivo assay may be attributed to saturation of microglia responses in 5-6 month old PS19 mice. Microglia activation is already detectable in 3 mo old PS19 mice and precedes astrogliosis.

Recently, Bridging Integrator 1 (BIN1), a known genetic risk factor for AD, was connected to tau seed release through exosomes in human AD and male PS19 mice. Levels of BIN1 in our F2 samples were analyzed. Neuronal BIN1 isoform 1 but not microglia specific isoform 2 were highly enriched in the F2 fractions (FIG. 10D). It was found that a high magnitude increase in exosome-associated BIN1 upon IL1β stimulation that was lower in the compound 11 treated group, but no statistically significant changes were found due to the high variability of individual levels of BIN1 within each group (FIG. 10D). This data suggests that nSMase2 and BIN1 could be a part of the same exosomal pathway responsible for tau release and spread in AD.

Overall, our rapid in vivo assay results demonstrate the effectiveness of novel dual AChE/nSMase2 inhibitor 11 in suppression of IL1β-induced release of tau-bearing exosomes in tauopathy model.

### Example 11: Parallel Artificial Membrane Permeability of Exemplary Compounds

To predict the potential for brain permeability, a Regis Technologies analytical column connected to an Agilent HPLC system (Infinity 1260 quaternary pump and multiple wavelength detector; 1200 autosampler) was used. An IAM.PC.DD column was (4.6 mm i.d.×10 cm, particle size 5 μm; pore size 300 Å) conditioned with 20 column volumes of mobile phase or until a stable line was achieved. A mobile phase with a consistent flow rate of 1 mL/min was used comprising 100 mM Na₂PO₄ (solvent A) and acetonitrile (solvent B); compounds were eluted using a gradient (min/% B: 0/30, 10/60, 11/30, 15/30). Detector settings were 220, 250 and 280 nm and acquisition time was 15 min. The autosampler injected 10 μL of compound at 2 mM final, diluted from 10 mM in DMSO in 70:30 (water:acetonitrile). After elution, compound retention and void volume times were obtained from the chromatogram. The IAM capacity factor: (K_{IAM}) was calculated using the equation:

$${K}_{IAM} = \frac{{t}_{r} - {t}_{0}}{{t}_{0}} ;$$

where tᵣ is the retention time of a compound and t₀ is the void volume time of the column. To predict CNS permeability, we used the correlation described by the formula below.

$$If \frac{{K}_{IAM}}{{MW}^{4}} \times {10}^{10} > 1.01 - CNS permiability is high \\ If \frac{{K}_{IAM}}{{MW}^{4}} \times {10}^{10} > 0.64 - CNS permiability is low$$

### Example 12: Preparation of Human Brain-Derived Synaptosomal Extracts

Brain autopsy samples were obtained from the University of California Irvine. Detailed information about individual cases is presented in Table 5. Brain tissue was cryopreserved and synaptosomal fractions (P2 fractions, ‘P2’) were prepared as previously described. In order to prepare P2 extracts, aliquots were quickly defrosted at 37° C. and centrifuged at 10,000 g for 10 minutes at 4° C. to remove P2 from sucrose. After aspirating the supernatant, cold PBS was added to each sample in a 1:5 weight/volume ratio. Samples were then sonicated in 10-second intervals three times, incubated on ice for 30 minutes and centrifuged at 20,000 g for 20 minutes at 4° C. P2 extracts were collected and stored at −80° C.

TABLE 5
Demographics for human cases selected
for synaptosomal extract preparation

| Brain | Age | PMI | | | |
|---|---|---|---|---|---|
| region | (years) | (hr) | Sex | Diagnosis | Stage |
| A40 | 55 | 4 | M | AD, CAA (occipital), MMSE | VI-C |
| | | | | 25 | |
| A40 | 65 | 5.3 | F | AD, CAA (frontal, occipital), | V-B |
| | | | | MMSE 7 | |
| A40 | 57 | 4 | F | AD, MMSE 11 | VI-C |

Table 4. Demographics for human cases selected for synaptosomal extract preparation for D+R and EMT assays. Disease stages were based on Braak neurofibrillary tangle score (I-VI) and CERAD Aβ plaque score (A-C). F—female; M—male; AD—Alzheimer's disease; CAA—cerebral amyloid angiopathy; PMI—postmortem interval; A40—area of parietal cortex, MMSE—mini-mental state examination.

### Example 13: Preparation of D+R and EMT Tau Biosensor-Based Assays

HEK293T Tau RD P301S FRET biosensor (tau biosensor) cells were grown in Dulbecco's Modified Eagle's medium (DMEM) with high glucose, 10% FBS, and 1% Penicillin-streptomycin at 37° C./5% CO₂. The lead inhibitors were tested in functional tau propagation “D+R” (Donors plus Recipients) and EMT (EV-mediated transfer) assays using the tau biosensors. Briefly, tau aggregation in donor cells (D) was induced by transfection of tau biosensor cells with human AD brain derived synaptosomal extracts (35 μg of synaptosomal fraction for 5 million cells in one 10 cm dish); control cells were treated with corresponding amount of transfection reagent lipofectamine 2000. After 24 hr incubation, the cells were trypsinized and plated together with DID-labeled recipient cells (R) in a 1:1 ratio in 96-well plates (15,000 of each cell type per well) for D+R assay or 1.5 million of donor cells per well in 6-well plates for EMT assay. Tested compounds or a corresponding amount of DMSO (0.2%) was added to the cell culture medium, DMEM, which contained 10% of exosome-depleted FBS (ThermoFisher, A2720803). Four technical replicates were used for each experimental condition. The inhibitors were tested at 20 μM concentration. In 48 hours 50 μL of culture medium per well were collected for cell viability test (Cytotox 96 non-radioactive cytotoxicity assay, Promega Corp., Madison, Wis.). The cells in D+R assay were harvested at the 48 hr time point and fixed with 2% paraformaldehyde for flow cytometry analysis according to published protocol. Tau seed transfer from donor to recipient cells results in FRET signal generation in the DID-positive recipient cells, which was measured by flow cytometry. Integrated FRET density was calculated in the top 30% of DID-positive cell based on DID signal intensity.

For the EMT assay, cell culture medium from donor cells growing for 48 hours with the compounds at concentrations of 20 μM or a corresponding amount of DMSO (0.2%), was collected and EVs were purified using the ExoQuick-TC kit (SBI biosciences) according to the manufacturer's instructions. Naïve recipient cells were plated in 96 well plates at a density of 20,000 cells per well in exosome-free medium and transfected with donor cell-derived exosomes at 12 hrs. The recipient cells were harvested 60 hours after transfection and FRET signals were analyzed using flow cytometry.

An Attune NxT Flow Cytometer (Invitrogen) equipped with an autosampler and FRET-compatible laser lines and filter sets was used for FRET signal detection. The FRET (CFP/YFP) signal was excited by a 405 nm laser for CFP excitation and detected in the YFP image detection channel. Flow cytometry data was analyzed using FCS Express version 5 software (DeNovo Software California, USA). Integrated FRET density was calculated as a product of percentage FRET positive cells and median of fluorescent intensity of the FRET positive cells as previously established.

### Example 14: Rapid In Vivo Assay and Brain EV Purification

ICV injections of IL-1β with or without pre-treatment with dual nSMase2/AChE inhibitor 8 or 11 were performed in 5-6 mo male PS19 mice expressing human tau with the P301S mutation under control of the murine prion promoter. Four groups (4 mice per group) were be used: group I (control) received SQ injection of vehicle (DMSO) and intracerebroventricular (ICV) injection of another vehicle (0.0006% BSA in PBS, pH7.4) one hour after SQ treatment; group II (IL1β) received SQ injection of vehicle and unilateral ICV injection of 0.2 ng of IL1β one hour later; group III (8/IL1β)—SQ treatment with 20 mk of 8 and ICV injection of 0.2 ng of IL1β; and group IV—SQ treatment with 20 mk of 11 and ICV injection of 0.2 ng of IL1β. Mice were euthanized by pentobarbital over-anesthesia, and perfused with cold PBS 2 hrs after IL-1β injection. Brains (minus cerebellum) were weighed and minced in ice-cold PBS and immediately processed for EV isolation. Brain EVs were isolated after gentle enzymatic and mechanical dissociation of the tissue using an adult brain dissociation kit and GentleMACS dissociator (Miltenyi). After cells and debris were filtered and pelleted by centrifugation, the supernatants were collected and the EV fraction purified by sequential differential and sucrose gradient rate-zonal ultracentrifugation, followed by a washing step. EV-contained pellets were resuspended in cryopreservation solution—25 mM trehalose solution in PBS, pH7.4 with protease and phosphatase inhibitor cocktail—then aliquoted and frozen at −80° C. This method of cryopreservation was shown to protect EVs from cryodamage and aggregation. The volume of the cryopreservation solution for each sample was calculated based on the weight if the brain sample used for EV isolation (0.4 g of tissue/150 μl of the solution).

### Example 15: Transmission Electron Microscopy (TEM)

For quality control purposes, small amounts of purified brain- or tissue culture-derived EVs were fixed on a copper mesh in glutaraldehyde/paraformaldehyde solution, stained with 2% uranyl acetate solution and imaged on a JEOL 100CX electron microscope at 29,000× magnification.

### Example 16: Immunoblot Analysis of EV Samples

Electrophoresis of proteins was performed using 10-20% Tris-Glycine gels in non-reducing (only for tetraspanins) or reducing (with addition of DTT) conditions; proteins were then transferred to PVDF membrane and probed with primary antibodies followed by HRP conjugated secondary antibodies. Chemiluminescent signals were generated with Super Signal West Femto substrate (Thermo Scientific Pierce 34095) and detected using a BioSpectrum 600 imaging system and quantified using VisionWorks Version 6.6A software (UVP; Upland, Calif.).

### Example 17: Statistical Analysis

All data was expressed as the mean±SEM. Significant differences were determined by one-way ANOVA followed by Bonferroni and Holm multiple comparison method using online web statistical calculator (http://astatsa.com/OneWay_Anova_with_TukeyHSD). Only a subset of pairs relative to the DMSO group were simultaneously compared. Values of * or ^{#}<0.05 and ** or ^{##}<0.01 were considered statistically significant.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound having a structure of Formula I: 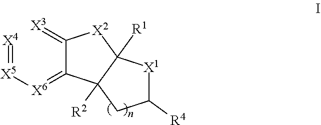
or a pharmaceutically acceptable salt thereof, wherein:
X¹ and X² are each independently O or NR^{a};
X³, X⁴, X⁵, and X⁶ are each independently CR³ or N;
R¹ is hydrogen or alkyl;
R² is alkyl, haloalkyl, alkoxy, cycloalkyl, aryl, aralkyl, or haloalkyl;
each R³ is independently selected from hydrogen, halogen, —OC(O)NR^{b}R^{c}, —NC(O)NR^{b}R^{c}, alkyl, haloalkyl, alkoxy, aralkyloxy, cycloalkyl, aryl, or heteroaryl;
R⁴ is hydrogen or alkyl;
R^{a} is hydrogen, alkyl, —C(O)-alkyl, —S(O)₂-alkyl, or —S(O)₂-aryl;
R^{b} is hydrogen, alkyl, cycloalkyl, aryl, heteroaryl, or heterocyclyl;
R^{c} is hydrogen or alkyl; and
n is an integer from 1 to 4.

2. The compound of claim 1, wherein the compound is not 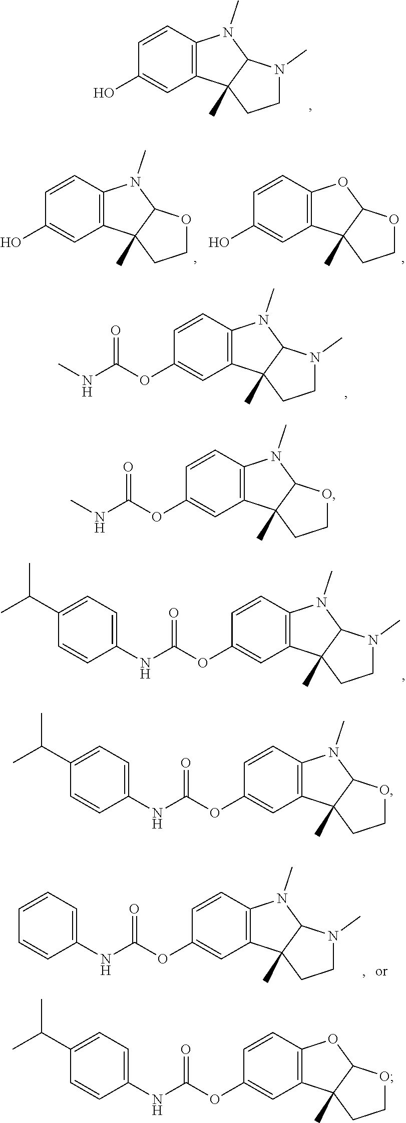
or a salt thereof.

3. The compound of claim 1, wherein the compound has a structure of Formula Ia or Ib: 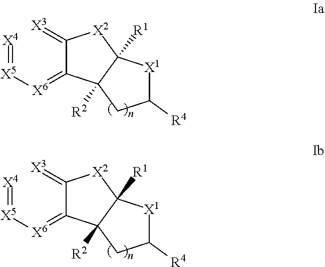
or a pharmaceutically acceptable salt thereof.

4. The compound of claim 1, wherein the compound has a structure of Formula IIa, IIb, or IIc: 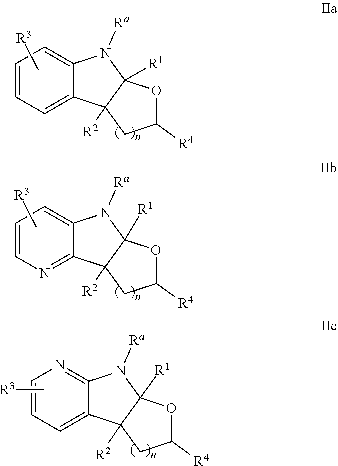
or a pharmaceutically acceptable salt thereof.

5. The compound of claim 4, wherein the compound has a structure of Formula IIIa, IIIb, or IIIc: 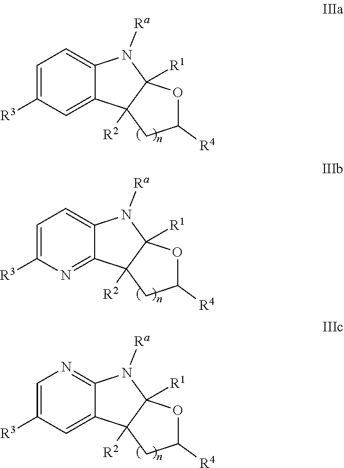
or a pharmaceutically acceptable salt thereof.

6. The compound of claim 1, wherein R¹ is hydrogen.

7. The compound of claim 1, wherein R² is C₁-C₄-alkyl or C₂-C₄-alkenyl.

8. The compound of claim 1, wherein R³ is aralkyloxy, bromo, chloro, aryl, —OC(O)NR^{b}R^{c}, —NC(O)NR^{b}R^{c}, or C₁-C₄-alkoxy.

9. The compound of claim 1, wherein at least one R^{a} is alkyl.

10. The compound of claim 9, wherein two R^{a} are alkyl.

11. The compound of claim 1, wherein R^{a} is hydrogen.

12. The compound of claim 1, wherein the compound has a structure of Formula IVa, IVb, or IVc: 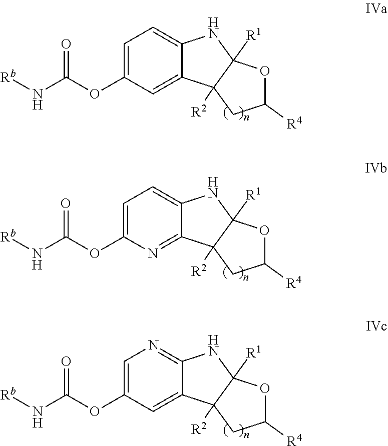
or a pharmaceutically acceptable salt thereof.

13. The compound of claim 1, wherein n is 1 or 2.

14. The compound of claim 1, wherein R⁴ is hydrogen.

15. (canceled)

16. The compound of claim 1, wherein the compound has a structure of Formula Va, Vb, or Vc: 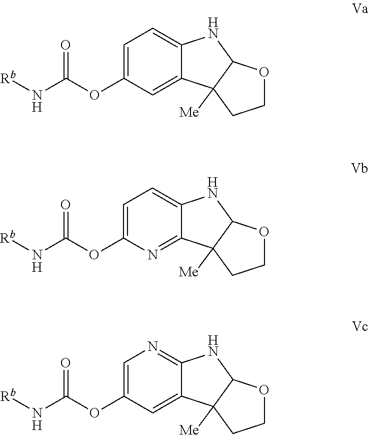
or a pharmaceutically acceptable salt thereof.

17. The compound of claim 1, wherein R^{b} is aryl.

18. A pharmaceutical composition comprising the compound of claim 1 and a pharmaceutically acceptable carrier or excipient.

19. (canceled)

20. (canceled)

21. A method for modulating acetylcholinesterase (AChE) or neutral sphingomyelinase 2 (n-SMase2) in a cell, comprising contacting a cell with a compound of claim 1.

22-24. (canceled)

25. A method for inhibiting the spread of Tau seeds from donor cells to recipient cells, comprising contacting the donor cells and/or the recipient with a compound of claim 1.

26. (canceled)

27. A method for treating or preventing a neurodegenerative disease or condition, comprising administering to a subject in need thereof a compound of claim 1.

28-30. (canceled)
